SEQUENCE LISTING (152 sequences)

SEQ ID NO: 1-4 - skipped (4 entries)

SEQ ID NO: 5 - protein, 10 aa, synthetic construct
SMCPLYKVHC

SEQ ID NO: 6 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACQWYGRMC

SEQ ID NO: 7 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SNCHWYGRIC

SEQ ID NO: 8 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SMCPLYKVHC

SEQ ID NO: 9 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SRCLFNWPDC

SEQ ID NO: 10 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
STCFLDVKNC

SEQ ID NO: 11 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACMLDVISC

SEQ ID NO: 12 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SGCMDDFAFC

SEQ ID NO: 13 - protein, 10 aa, synthetic construct
SDCYPDTYHC

SEQ ID NO: 14 - protein, 10 aa, synthetic construct
  DISULFID 3..10: note = Disulfide bond links residue 3 to residue 10
SDCYPDTYHC

SEQ ID NO: 15 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SECPWPLPQC

SEQ ID NO: 16 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDTYHC

SEQ ID NO: 17 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SHCKLDIWVC

SEQ ID NO: 18 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SHCKLDIWVC

SEQ ID NO: 19 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCHWYGRSC

SEQ ID NO: 20 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCLGWEQTC

SEQ ID NO: 21 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACQWYGRMC

SEQ ID NO: 22 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SFCLGYEASC

SEQ ID NO: 23 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDIPTC

SEQ ID NO: 24 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDIPTC

SEQ ID NO: 25 - protein, 9 aa, synthetic construct
  CROSSLNK 1..9: note = O-(6-(3,5-bis(chloromethyl)phenoxy)hexyl)hydroxylam ine links residue 1 to residue 3 and residue 9
SACRSDTLC

SEQ ID NO: 26 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPLDVPSC

SEQ ID NO: 27 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SECYPDTYHC

SEQ ID NO: 28 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCFPDTYHC

SEQ ID NO: 29 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDTFHC

SEQ ID NO: 30 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SICYPDTYHC

SEQ ID NO: 31 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
STCHEMNWKPC

SEQ ID NO: 32 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
STCIYGKFTC

SEQ ID NO: 33 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
STCGPESEKDC

SEQ ID NO: 34 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SQCGPVKWGHC

SEQ ID NO: 35 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SWCTKATKNAC

SEQ ID NO: 36 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SHCRIMEVEQC

SEQ ID NO: 37 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SMCSYMNTIC

SEQ ID NO: 38 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SECQENLKYC

SEQ ID NO: 39 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACYPDTYHC

SEQ ID NO: 40 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCAPDTYHC

SEQ ID NO: 41 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYADTYHC

SEQ ID NO: 42 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPATYHC

SEQ ID NO: 43 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDAYHC

SEQ ID NO: 44 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDTAHC

SEQ ID NO: 45 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDTYAC

SEQ ID NO: 46 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACPMDIPTC

SEQ ID NO: 47 - protein, 10 aa, synthetic construct
  CROSSLNK 1..10: note = note = O-(6-(3,5-bis(chloromethyl)phenoxy)hexyl)hydroxylamine links residue 1 to residue 3 and residue 10
SQCAMDIPTC

SEQ ID NO: 48 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPADIPTC

SEQ ID NO: 49 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMAIPTC

SEQ ID NO: 50 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDAPTC

SEQ ID NO: 51 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDIATC

SEQ ID NO: 52 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDIPAC

SEQ ID NO: 53 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACPLDVPSC

SEQ ID NO: 54 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCALDVPSC

SEQ ID NO: 55 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPADVPSC

SEQ ID NO: 56 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPLAVPSC

SEQ ID NO: 57 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPLDAPSC

SEQ ID NO: 58 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPLDVASC

SEQ ID NO: 59 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPLDVPAC

SEQ ID NO: 60 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCHDKFTEC

SEQ ID NO: 61 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCHDPPKVC

SEQ ID NO: 62 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SDCPDSMAHSC

SEQ ID NO: 63 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SDCPDAERYIC

SEQ ID NO: 64 - protein, 8 aa, synthetic construct
  CROSSLNK 1..6: note = 3-chloropropanoic acid links residue 1 to residue 6
RSDTLCWK

SEQ ID NO: 65 - protein, 8 aa, synthetic construct
  SITE 6: note = X - Modified with pencillamine at this residue
  CROSSLNK 1..6: note = 2-(chloromethyl)benzoic acid links residue 1 to residue 6
RSDTLXWE

SEQ ID NO: 66 - protein, 9 aa, synthetic construct
  DISULFID 1..7: note = Disulfide bond links residue 1 to residue 7
CRSDTLCGE

SEQ ID NO: 67 - protein, 7 aa, synthetic construct
  DISULFID 1..7: note = Disulfide bond links residue 1 to residue 7
CRSDTLC

SEQ ID NO: 68 - protein, 10 aa, synthetic construct
  CROSSLNK 1..10: note = O-(6-(3,5-bis(chloromethyl)phenoxy)hexyl)hydroxylam ine links residue 1 to residue 3 and residue 9
SACRSDTLCG

SEQ ID NO: 69 - protein, 7 aa, synthetic construct
  DISULFID 1..7: note = Disulfide bond links residue 1 to residue 7
CRSDTLC

SEQ ID NO: 70 - protein, 9 aa, synthetic construct
  CROSSLNK 1..9: note = O-(3,5-bis(chloromethyl)benzyl)hydroxylamine links residue 1 to residue 3 and residue 9
SACRSDTLC

SEQ ID NO: 71 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPADTPYEC

SEQ ID NO: 72 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLATPYEC

SEQ ID NO: 73 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SHCKLDIWVC

SEQ ID NO: 74 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
STCRMDFQDC

SEQ ID NO: 75 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SECPWPLPQC

SEQ ID NO: 76 - protein, 15 aa, synthetic construct
  NON_STD 4: note = X - Modified with norleucine at this residue
  DISULFID 2..13: note = Disulfide bond links residue 2 to residue 13
  CROSSLNK 1..7: note = Thioether bond links residue 1 to residue 7
  NON_STD 10: note = X - Modified with norleucine at this residue
RCSXDICRSXDICWQ

SEQ ID NO: 77 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SFCQMFEKAC

SEQ ID NO: 78 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SMCYPDTYHC

SEQ ID NO: 79 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SFCGATMKKC

SEQ ID NO: 80 - protein, 10 aa, synthetic construct
  NON_STD 5: note = X - Modified with methionine sulfoxide at this residue
  CROSSLNK 1..10: note = O-(6-(3,5-bis(chloromethyl)phenoxy)hexyl)hydroxylam ine links residue 1 to residue 3 and residue 10
SQCPXAIPTC

SEQ ID NO: 81 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SFCLGYEASC

SEQ ID NO: 82 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCYPDFYHC

SEQ ID NO: 83 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCWPDTYHC

SEQ ID NO: 84 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SRCLDIMYDC

SEQ ID NO: 85 - protein, 9 aa, synthetic construct
  CROSSLNK 1..9: note = O-(6-(3,5-bis(chloromethyl)phenoxy)hexyl)hydroxylam ine links residue 1 to residue 3 and residue 9
SACRSDTLC

SEQ ID NO: 86 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SNCYPDTYHC

SEQ ID NO: 87 - protein, 15 aa, synthetic construct
  DISULFID 2..13: note = Disulfide bond links residue 2 to residue 13
  CROSSLNK 2..6: note = Thioether bond links residue 2 to residue 6
RCSDTLCRSLDVCWQ

SEQ ID NO: 88 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACSDTGLQC

SEQ ID NO: 89 - protein, 11 aa, synthetic construct
  NON_STD 5: note = X - Modified with O-methyl homoserine at this residue
  SITE 7: note = X - Modified with tert-butyl alanine at this residue
  CROSSLNK 1..11: note = 7-(3,5-bis(chloromethyl)phenoxy)heptanamide links residue 1 to residue 3 and residue 11
GQCPXDXPQEC

SEQ ID NO: 90 - protein, 15 aa, synthetic construct
  NON_STD 4: note = X - Modified with norleucine at this residue
  DISULFID 2..13: note = Disulfide bond links residue 2 to residue 13
  CROSSLNK 2..7: note = Thioether bond links residue 2 to residue 7
RCSXDICRSDTLCWQ

SEQ ID NO: 91 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SACPMDVWGEC

SEQ ID NO: 92 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SLCYPDTYHC

SEQ ID NO: 93 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SMCPLYIVHC

SEQ ID NO: 94 - protein, 15 aa, synthetic construct
  NON_STD 4: note = X - Modified with norleucine at this residue
  DISULFID 2..13: note = Disulfide bond links residue 2 to residue 13
  CROSSLNK 2..7: note = Thioether bond links residue 2 to residue 7
RCSXDICRSLDVCWQ

SEQ ID NO: 95 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCPWQSKTC

SEQ ID NO: 96 - protein, 15 aa, synthetic construct
  NON_STD 10: note = X - Modified with norleucine at this residue
  DISULFID 2..13: note = Disulfide bond links residue 2 to residue 13
  CROSSLNK 2..7: note = Thioether bond links residue 2 to residue 7
RCSDTLCRSXDICWQ

SEQ ID NO: 97 - protein, 10 aa, synthetic construct
  NON_STD 5: note = X - Modified with methionine sulfoxide at this residue
  CROSSLNK 1..10: note = O-(6-(3,5-bis(chloromethyl)phenoxy)hexyl)hydroxylam ine links residue 1 to residue 3 and residue 10
SQCPXDAPTC

SEQ ID NO: 98 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SICRTDVPIDC

SEQ ID NO: 99 - protein, 10 aa, synthetic construct
  NON_STD 5: note = X - Modified with norleucine at this residue
  CROSSLNK 1..10: note = O-(6-(3,5-bis(chloromethyl)phenoxy)hexyl)hydroxylam ine links residue 1 to residue 3 and residue 10
SQCPXDIATC

SEQ ID NO: 100 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SLCYEWEHAC

SEQ ID NO: 101 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SACPMDVAGEC

SEQ ID NO: 102 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SDCHDKFTEC

SEQ ID NO: 103 - protein, 11 aa, synthetic construct
  NON_STD 5: note = X - Modified with tert-butyl alanine at this residue
  CROSSLNK 1..11: note = O-(6-(3,5-bis(chloromethyl)phenoxy)hexyl)hydroxylam ine links residue 1 to residue 3 and residue 11
SRCPXDVWGEC

SEQ ID NO: 104 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SACAWHQHAC

SEQ ID NO: 105 - protein, 11 aa, synthetic construct
  NON_STD 5: note = X - Modified with cyclobutyl alanine at this residue
  CROSSLNK 1..11: note = O-(6-(3,5-bis(chloromethyl)phenoxy)hexyl)hydroxylam ine links residue 1 to residue 3 and residue 11
  NON_STD 7: note = X - Modified with tert-butyl alanine at this residue
SRCPXDXWQEC

SEQ ID NO: 106 - protein, 11 aa, synthetic construct
  NON_STD 5: note = X - Modified with norleucine at this residue
  NON_STD 7: note = X - Modified with cyclobutyl alanine at this residue
  CROSSLNK 1..11: note = O-(6-(3,5-bis(chloromethyl)phenoxy)hexyl)hydroxylam ine links residue 1 to residue 3 and residue 11
SRCPCDCWQEC

SEQ ID NO: 107 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SACPMDVWQEC

SEQ ID NO: 108 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCALDTPYEC

SEQ ID NO: 109 - protein, 12 aa, synthetic construct
  NON_STD 1: note = X - Modified with N-Acetylserine at this residue
  DISULFID 3..11: note = Disulfide bond links residue 3 to residue 11
  CROSSLNK 12: note = polyethylene glycol5 links residue 12 to residue 12 of another copy of the sequence to create a dimer
XRCPMDIAGECK

SEQ ID NO: 110 - protein, 11 aa, synthetic construct
  NON_STD 5: note = X - Modified with O-methyl homoserine at this residue
  CROSSLNK 1..11: note = O-(6-(3,5-bis(chloromethyl)phenoxy)hexyl)hydroxylam ine links residue 1 to residue 3 and residue 11
SRCPXDIWYEC

SEQ ID NO: 111 - protein, 10 aa, synthetic construct
  NON_STD 1: note = X - Modified with N-acetylserine at this residue
  DISULFID 3..10: note = Disulfide bond links residue 3 to residue 10
XQCPMDIPTC

SEQ ID NO: 112 - protein, 11 aa, synthetic construct
  NON_STD 5: note = X - Modified with norleucine at this residue
  SITE 7: note = X - Modified with cyclobutyl alanine at this residue
  CROSSLNK 1..11: note = 7-(3,5-bis(chloromethyl)phenoxy)heptanamide links residue 1 to residue 3 and residue 11
GKCPXDXWQEC

SEQ ID NO: 113 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SACPLDTPYEC

SEQ ID NO: 114 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDAPYEC

SEQ ID NO: 115 - protein, 10 aa, synthetic construct
  DISULFID 3..10: note = Disulfide bond linking residue 3 to residue 10
SKCPLDVPSC

SEQ ID NO: 116 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDIPTC

SEQ ID NO: 117 - protein, 11 aa, synthetic construct
  NON_STD 5: note = X - Modified with norleucine at this residue
  NON_STD 7: note = X - Modified with tert-butyl alanine at this residue
  CROSSLNK 1..11: note = O-(6-(3,5-bis(chloromethyl)phenoxy)hexyl)hydroxylam ine links residue 1 to residue 3 and residue 11
SRCPXDXPYEC

SEQ ID NO: 118 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTAYEC

SEQ ID NO: 119 - protein, 11 aa, synthetic construct
  NON_STD 1: note = X - Modified with N-acetylserine at this residue
  DISULFID 3..11: note = Disulfide bond links residue 3 to residue 11
XRCPMDIAGEC

SEQ ID NO: 120 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTPAEC

SEQ ID NO: 121 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTPTEC

SEQ ID NO: 122 - protein, 11 aa, synthetic construct
  NON_STD 5: note = X - Modified with norleucine at this residue
  CROSSLNK 1..11: note = 7-(3,5-bis(chloromethyl)phenoxy)heptanamide links residue 1 to residue 3 and residue 11
GACPXDVWQEC

SEQ ID NO: 123 - protein, 12 aa, synthetic construct
  NON_STD 1: note = X - Modified with N-acetylserine at this residue
  DISULFID 3..11: note = Disulfide bond links residue 3 to residue 11
  CROSSLNK 12: note = 2,2'-oxydiacetic acid links residue 12 to residue 12 of another copy of the sequence to create a dimer
XRCPMDIAGECK

SEQ ID NO: 124 - protein, 11 aa, synthetic construct
  NON_STD 5: note = X - Modified with tert-butyl alanine at this residue
  CROSSLNK 1..11: note = O-(6-(3,5-bis(chloromethyl)phenoxy)hexyl)hydroxylam ine links residue 1 to residue 3 and residue 11
SRCPXDVPQEC

SEQ ID NO: 125 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDMPYEC

SEQ ID NO: 126 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SQCPMDVPTC

SEQ ID NO: 127 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDIWVKC

SEQ ID NO: 128 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTPQEC

SEQ ID NO: 129 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTPYAC

SEQ ID NO: 130 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SRCPMDVAGEC

SEQ ID NO: 131 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDTPYEC

SEQ ID NO: 132 - protein, 11 aa, synthetic construct
  NON_STD 5: note = X - Modified with norleucine at this residue
  CROSSLNK 1..11: note = 7-(3,5-bis(chloromethyl)phenoxy)heptanamide links residue 1 to residue 3 and residue 11
GKCPXDIWVKC

SEQ ID NO: 133 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SRCPMDIPTC

SEQ ID NO: 134 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDLPYEC

SEQ ID NO: 135 - protein, 10 aa, synthetic construct
  CROSSLNK 1..10: note = O-(6-(3,5-bis(chloromethyl)phenoxy)hexyl)hydroxylam ine links residue 1 to residue 3 and residue 10
SKCPMDIPTC

SEQ ID NO: 136 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SRCPLDTPYEC

SEQ ID NO: 137 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SRCPMDIWQEC

SEQ ID NO: 138 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDVAGEC

SEQ ID NO: 139 - protein, 11 aa, synthetic construct
  NON_STD 5: note = X - Modified with O-methyl homoserine at this residue
  CROSSLNK 1..11: note = O-(6-(3,5-bis(chloromethyl)phenoxy)hexyl)hydroxylam ine links residue 1 to residue 3 and residue 11
SRCPXDIWQEC

SEQ ID NO: 140 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SRCPMDIAGEC

SEQ ID NO: 141 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDIAAEC

SEQ ID NO: 142 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDIPYEC

SEQ ID NO: 143 - protein, 11 aa, synthetic construct
  CROSSLNK 1..11: note = O-(6-(3,5-bis(chloromethyl)phenoxy)hexyl)hydroxylam ine links residue 1 to residue 3 and residue 11
SKCPLDVPYEC

SEQ ID NO: 144 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDIWQEC

SEQ ID NO: 145 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDVWYEC

SEQ ID NO: 146 - protein, 7 aa, synthetic construct
  NON_STD 1: note = X - Modified with N-acetylcysteine at this residue
  DISULFID 1..7: note = Disulfide bond linking residue 1 to residue 7
XRSDTLC

SEQ ID NO: 147 - protein, 11 aa, synthetic construct
  NON_STD 5: note = X - Modified with norleucine at this residue
  CROSSLNK 1..11: note = 7-(3,5-bis(chloromethyl)phenoxy)heptanamide links residue 1 to residue 3 and residue 11
GRCPXDIWQEC

SEQ ID NO: 148 - protein, 11 aa, synthetic construct
  NON_STD 5: note = X - Modified with norleucine at this residue
  CROSSLNK 1..11: note = O-(6-(3,5-bis(chloromethyl)phenoxy)hexyl)hydroxylam ine links residue 1 to residue 3 and residue 11
SRCPXDVWQEC

SEQ ID NO: 149 - protein, 8 aa, synthetic construct
  NON_STD 1: note = X - Modified with N2-methylarginine at this residue
  CROSSLNK 1..6: note = 3-chloropropanoic acid links residue 1 to residue 6
XSDTLCWK

SEQ ID NO: 150 - protein, 8 aa, synthetic construct
  NON_STD 1: note = X - Modified with N2-methylarginine at this residue
  SITE 6: note = X - Modified with penicillamine at this residue
  CROSSLNK 1..6: note = 2-(chloromethyl)benzoic acid links residue 1 to residue 6
XSDTLXWE

SEQ ID NO: 151 - protein, 9 aa, synthetic construct
  NON_STD 1: note = X - Modified with N-acetylcysteine at this residue
  DISULFID 1..9: note = Disulfide bond linking residue 1 to residue 9
XRSDTLCGE

SEQ ID NO: 152 - protein, 11 aa, synthetic construct
  DISULFID 3..11: note = Disulfide bond linking residue 3 to residue 11
SKCPLDTPYEC

(19) United States
(12) Patent Application Publication
(10) Pub. No.: US 2023/0145835 A1
(43) Pub. Date: May 11, 2023

(54) Modulators of Alpha-4-beta-7 Integrin and MAdCAM

(71) Applicant: 48HD Biopharma Inc., Edmonton (CA)

(72) Inventors: John J. DWYER, Ramona, CA (US); Jessica CAO, Edmonton (CA)

(21) Appl. No.: 17/938,236

(22) Filed: Oct. 5, 2022

Related U.S. Application Data
(60) Provisional application No. 63/253,078, filed on Oct. 6, 2021.

(51) Int. Cl. C07D 417/12; C07D 515/18; C07D 285/00; C07D 515/22; C07D 515/14; C07D 519/00; C07D 515/08

(52) U.S. Cl. CPC C07D 417/12; C07D 515/18; C07D 285/00; C07D 515/22; C07D 515/14; C07D 519/00; C07D 515/08

(57) ABSTRACT

The instant disclosure describes novel modulators of alpha α4β7 and/or MAdCAM and their use for the treatment of diseases and conditions associated with their biological function. Further described herein are methods of treating diseases or conditions associated with α4β7 and/or MAdCAM.

### STATEMENT REGARDING SEQUENCE LISTING

The Sequence Listing associated with this application is provided in text format in lieu of paper copy, and is hereby incorporated by reference into the specification. The name of the XML file containing the Sequence Listing is “83928-366985 Sequence Listing_ST26.” The XML file is 209 KB, was created on Oct. 4, 2022, and is being submitted electronically via EFS-Web, concurrent with the filing of this specification.

### FIELD OF THE INVENTION

The present disclosure relates to the novel compounds and their use for the treatment of symptoms or pathological sequelae associated with the biological function of adhesion molecule α4β7 integrin and/or Mucosal Vascular Addressin Cell Adhesion molecule (MAdCAM).

### BACKGROUND OF THE INVENTION

Integrins represent an important class of cell-adhesion molecules involved in cell-cell and cell-extracellular matrix communication. Unlike other cell surface receptors that bind their target ligand with high affinity, integrins differ in that they bind their target ligand with lower binding affinity despite having higher cell surface concentration as compared to cell surface receptors. Thus, not only do integrins facilitate the linkage between the cell cytoskeleton and the extracellular matrix, but the resulting weak adhesion over a large population of receptors bound to ligand allows for the reversible attachment of cells to the extracellular matrix, or the “Velcro principle” of cell adhesion. Alberts et al., 2002, herein incorporated by reference with regard to such background teaching.

Integrins are heterodimeric transmembrane proteins comprised of an a and p subunit. In mammals, 18 α subunit and 8 β subunit genes have been identified, resulting in at least 24 different combinations of α-β heterodimers. Humphries et al., 2006, herein incorporated by reference with regard to such background teaching. Each subunit is comprised of several domains and flexible linkers arranged to form an extracellular domain, a single, membrane-spanning helix, and a cytoplasmic tail. Thus, integrins assume an overall “head” and “two legs” structure, where the head is the primary location of ligand binding. Campbell et al., 2011, herein incorporated by reference with regard to such background teaching. External stimulation by divalent cations facilitates integrin binding to extracellular ligands. Binding of target ligands in turn results in conformational changes that alters the intracellular domains, which impact cell signaling cascades. Tsimbouri et al., 2014, herein incorporated by reference with regard to such background teaching. The heterodimer structure is bent or “closed” in its resting state, and takes on an “open” configuration after activation.

The integrin α4β7 is highly expressed on T and B cells and other leucocytes. Thus, α4β7 is involved in the immune response by recruiting lymphocytes from the blood to gut associated lymphoid tissue. Zhang et al., 2020, herein incorporated by reference with regard to such background teaching. Mucosal vascular addressin cell adhesion molecule-1 (MAdCAM-1 or “MAdCAM”) is the predominant ligand for α4β7, and is expressed on mucosal endothelial cells and venules and steers lymphocytes from circulation across the vascular endothelial barrier into the intestinal lamina. Berlin et al., 1993, herein incorporated by reference with regard to such background teaching. Thus, the mobilization of lymphocytes to gut tissue is mediated, at least in part, by the interaction of α4β7 with MAdCAM. Rose et al., 2007 and Erie et al., 1994, herein incorporated by reference with regard to such background teaching. Overexpression of MAdCAM and the resulting influx of α4β7 is believed to be a contributor to intestinal inflammation issues. Soler-Ferran et al., 2012, herein incorporated by reference with regard to such background teachings.

However, in certain cases, excess recruitment of leukocytes to the gut may occur. This uncontrolled immune response is a characteristic of certain gut-associated diseases such as inflammatory bowel disease, ulcerative colitis, and Crohn's disease. Symptoms may include epithelial damage, reduction in microbial diversity, aberrant lymphocyte activation, and increased expression of various cytokines. Lamb et al., 2018, herein incorporated by reference with regard to such background teachings. Chronic inflammation results in patients suffering from a host of symptoms including digestive pain, diarrhea, rectal bleeding, fatigue, and weight loss. Furthermore, gut immunity is linked to extra-intestinal autoimmune disorders, such as Multiple Sclerosis (MS). Kuhbandner et al., 2019, herein incorporated by reference with regard to such background teachings.

Thus, there is a need for therapeutics directed to modulating α4β7 and MAdCAM in the gut and treatments for diseases or conditions associated with their biological function.

### SUMMARY OF THE INVENTION

In one embodiment described herein is a compound comprising a peptide scaffold and one or more of a linker, a sulfide or disulfide bond, wherein the linker, sulfide, or disulfide bond is covalently bonded to the peptide scaffold. In one aspect, the peptide scaffold comprises a sequence of Formula (I):

XY¹Y²X′ₘZ¹Z²Z³Z⁴Z⁵

or a pharmaceutically acceptable salt thereof, wherein
X is Ser, Cys, Gly, Arg, R(NMe), Ac-Ser, or Ac-Cys;
Y¹ is any amino acid;
Y² is Cys, Ser, or Asp;
each X′ independently is any amino acid;
Z¹ is Cys, Asp, Thr, or absent;
Z² is Leu, Ile, Val, Lys, or absent;
Z³ is Cys or absent;
Z⁴ is Trp or absent;
Z⁵ is Gln or absent; and
m is 4, 5, 6, or 7 and each X′ may be the same or different.

In another aspect
Y¹ is Gln, Lys, His, Thr, Glu, Cys, Phe, Met, Asp, Ala, Arg, Ile, Asn, Gly, Leu, Ser, or Trp;
each X′ independently is His, Pro, Lys, Arg, Phe, Nle, Gln, Tyr, Gly, Leu, Trp,
Asp, Ser, Met, Ala, Ile, Thr, Ox-Met, Hse(OMe), Glu, tBuAla, CbA, Asn, Val, Cys, or Pen.

In one aspect the peptide scaffold comprises a sequence of Formula (III):

XY¹Y²X′ₘZ¹Z²

or a pharmaceutically acceptable salt thereof, wherein
X is Ser, Cys, Gly, R(NMe), Ac-Ser, or Ac-Cys;
Y¹ is Gln, Leu, Asp, Lys, Ala, Arg, or Ser;
Y² is Cys, Ser, or Asp;
each X′ independently is His, Pro, Asp, Met, Thr, Nle, Hse(OMe), Ile, Val, Pen, Cys, Trp, Ala, Gly, Tyr, Gln, Glu, Leu, Ser, Cys, Phe, Arg, tBuAla, CbA, Lys, or absent;
Z¹ is Cys or absent;
Z² is Lys or absent; and
m is 4, 5, 6, or 7 and each X′ may be the same or different.

In one aspect the peptide scaffold comprises a sequence of Formula (IV):

XY¹Y²X′ₘZ¹

or a pharmaceutically acceptable salt thereof, wherein
X is Ser, Cys, Gly, R(NMe), or Ac-Cys;
Y¹ is Lys, Arg, or Ser;
Y² is Cys, Ser, or Asp;
each X′ independently is Pro, Asp, Met, Leu, Thr, Nle, Hse(OMe), Ile, Val, Pen, Cys, Trp, Ala, Gly, Tyr, Gln, Glu, Lys, or absent;
Z¹ is Cys or absent; and
m is 4, 5, 6, or 7 and each X′ may be the same or different.

In another aspect the linker comprises DIG, PEG, IDA, ADA, Boc-IDA, Glutaric acid, Isopthalic acid, 1,3-phenylenediacetic acid, 1,4-phenylenediacetic acid, 1,2-phenylenediacetic acid, Triazine, Boc-Triazine, IDA-biotin, PEG4-biotin, AADA, any suitable aliphatics, aromatics, heteroaromatics, polyethylene glycol, two-fold symmetric linchpin (TSL), TSLC, PFS, TBMB, DBMB, chloroacetic acid, 3-chloropropanoic acid, or 2-(chloromethyl)benzoic acid.

In another aspect the linker comprises DIG, PEG, TSL, TSLC, chloroacetic acid, 3-chloropropanoic acid, or 2-(chloromethyl)benzoic acid.

In one aspect the X is Ser; the linker is a TSL; and wherein the linker is bound to the peptide scaffold at the Ser by an aldehyde reactive oxime functionality.

In another aspect the X is Gly; the linker is a TSLC; and wherein the linker is bound to the peptide scaffold at the Gly by an amide functionality.

In one aspect the X is R(NMe); the linker is 3-chloropropanoic acid (Pa) or 2-(chloromethyl)benzoic acid (Bz); and wherein the linker is bound to the peptide scaffold at the R(NMe) by an amide functionality.

In one aspect the X is R; the linker is chloroacetic acid; and wherein the linker is bound to the peptide scaffold at the R by an amide functionality.

Another aspect described herein is a dimer, comprising a first and a second peptide scaffold as described herein, wherein a DIG or PEG linker is bound to the first peptide scaffold at Z² by an amide functionality, and bound to the second peptide scaffold at Z² by an amide functionality, and wherein Z² is Lys.

In one aspect the compound comprises a disulfide bond covalently linking Y¹ and Z³, X and X′, Y² and X′, or Y² and Z¹, wherein Y¹, Z³, X, X′, Y² and Z¹ is Cys, X and X′ are separated by 5, 6, or 7 amino acids, and Y² and X′ are separated by 5, 6, or 7 amino acids.

In one aspect the X and X′ are separated by 5 amino acids and Y² and X′ are separated by 6 or 7 amino acids.

In one aspect the linker is bound to the peptide scaffold at X′ by a thioether functionality, wherein X′ is Pen.

In one aspect the linker is bound to the peptide scaffold at X′ by a thioether functionality, wherein X′ is Cys.

In one aspect the linker is further bound at Y² and either X′ or Z¹, each by a thiol-reactive chlorobenzyl functionality, wherein Y² is Cys, X′ is Cys, and Z¹ is Cys, and 5, 6, or 7 amino acids separate Y² and X′ or Z¹.

In one aspect the linker comprises a two-fold symmetric linchpin (TSL) linker or a TSLC linker.

In one aspect the linker comprises TSL-1, TSL-3, TSL-6 or TSLC-7.

In one aspect the peptide scaffold comprises the sequences of Table 2.

Another embodiment described herein is a compound selected from the group consisting of scaffold A, B, C, D, E, F, G, H I, and J, wherein each X independently is Ser, Cys, Gly, Arg, R(NMe), Ac-Ser, or Ac-Cys;
each Y¹ independently is any amino acid;
each Y² independently is any amino acid;
each X′ independently is any amino acid;
each X″ independently is any amino acid or absent;
each C′″ independently is Cys or an Ac-Cys;
each X′″ independently is any amino acid;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In one aspect
each X independently is Ser, Cys, Gly, Arg, R(NMe), Ac-Ser, or Ac-Cys;
each Y¹ independently is Gln, Lys, His, Thr, Glu, Cys, Phe, Met, Asp, Ala, Arg, Ile, Asn, Gly, Leu, Ser, or Trp;
each Y² independently is Cys, Ser, or Asp;
each X′ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;
each X″ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, Pen, or absent;
each C′″ independently is Cys or an Ac-Cys;
each X′″ independently is Ser, Cys, Gly, Arg, R(NMe), Ac-Ser, or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent; and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In one aspect the each X independently is Ser, Cys, Gly, Arg, R(NMe), Ac-Ser, or Ac-Cys;
each Y¹ independently is Lys, Arg, or Ser;
each Y² independently is Cys, Ser, or Asp;
each X′ independently is Pro, Asp, Met, Leu, Thr, Nle, Hse(OMe), Ile, Val, Pen, Cys, Trp, Ala, Gly, Tyr, Gln, Glu, Lys, or absent;
each X″ independently is Pro, Asp, Met, Leu, Thr, Nle, Hse(OMe), Ile, Val, Pen, Cys, Trp, Ala, Gly, Tyr, Gln, Glu, Lys, or absent;
each C′″ independently is Cys or an Ac-Cys;
each X′″ independently is Ser, Cys, Gly, R(NMe), or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

Another embodiment described herein is a compound selected from the group consisting of scaffold A-S, B-S, C-S, D-S, E-S, F-S, G-S, H-S, I-S, and J-S, wherein each X independently is Ser, Cys, Gly, Arg, R(NMe), Ac-Ser, or Ac-Cys;
each Y¹ independently is any amino acid;
each Y² independently is any amino acid;
each X′ independently is any amino acid;
each X″ independently is any amino acid or absent;
each C′″ independently is Cys or an Ac-Cys;
each X′″ independently is any amino acid;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In one aspect

each X independently is Ser, Cys, Gly, Arg, R(NMe), Ac-Ser, or Ac-Cys;

each Y¹ independently is Gln, Lys, His, Thr, Glu, Cys, Phe, Met, Asp, Ala, Arg, Ile, Asn, Gly, Leu, Ser, or Trp;

each Y² independently is Cys, Ser, or Asp;

each X′ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;

each X″ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, Pen, or absent;

each C′″ independently is Cys or an Ac-Cys;

each X′″ independently is Ser, Cys, Gly, Arg, R(NMe), Ac-Ser, or Ac-Cys;

each Z¹ independently is Cys, Asp, or Thr;

each Z² independently is Leu, Ile, Val, or Lys;

each Z⁴ independently is Trp or absent;

each Z⁵ independently is Gln or absent; and

each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In one aspect each X independently is Ser, Cys, Gly, Arg, R(NMe), Ac-Ser, or Ac-Cys;
each Y¹ independently is Lys, Arg, or Ser;
each Y² independently is Cys, Ser, or Asp;
each X′ independently is Pro, Asp, Met, Leu, Thr, Nle, Hse(OMe), Ile, Val, Pen, Cys, Trp, Ala, Gly, Tyr, Gln, Glu, Lys, or absent;
each X″ independently is Pro, Asp, Met, Leu, Thr, Nle, Hse(OMe), Ile, Val, Pen, Cys, Trp, Ala, Gly, Tyr, Gln, Glu, Lys, or absent;
each C′″ independently is Cys or an Ac-Cys;
each X′″ independently is Ser, Cys, Gly, R(NMe), or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In one aspect any of the compounds described herein, wherein the compound is bicyclic.

Another embodiment described herein is a compound selected from the group consisting of:

| No. | Structure |
|---|---|
| 1 | 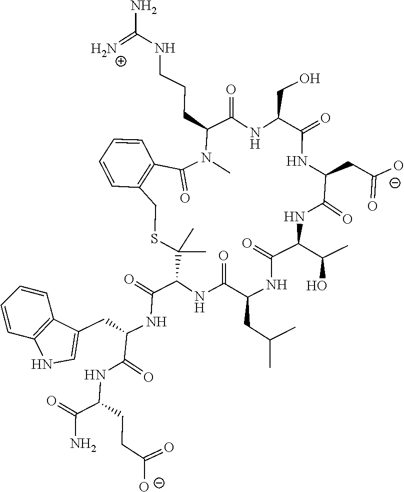 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)c2ccccc2CSC(C)(C)[C@@H](C(=O)N[C@@H](Cc2c[nH]c3ccccc23)C(=O)N[C@H](CCC(=O)[O-])C(N)=O)NC1=O] |
| 2 | 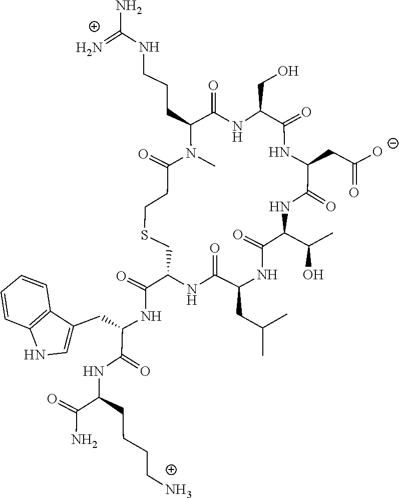 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)CCSC[C@@H](C(=O)N[C@@H](Cc2c[nH]c3ccccc23)C(=O)N[C@@H](CCCC[NH3+])C(N)=O)NC1=O] |
| 3 | 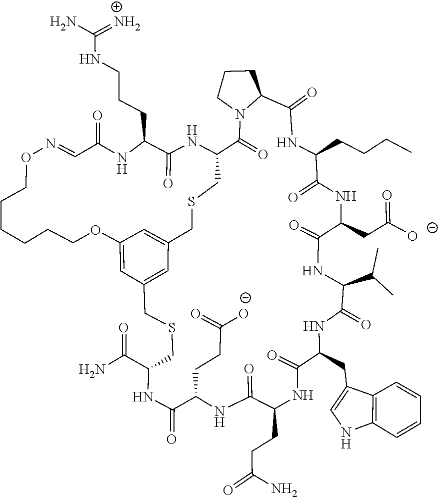 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 4 | 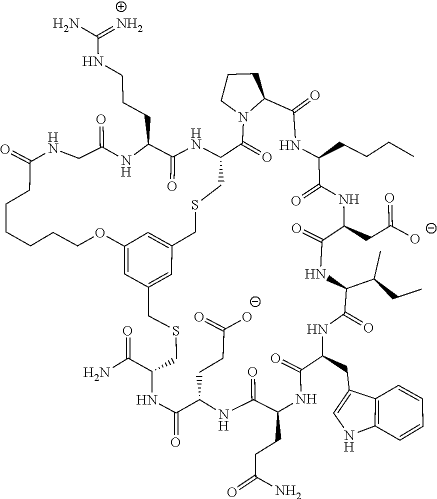 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 5 | 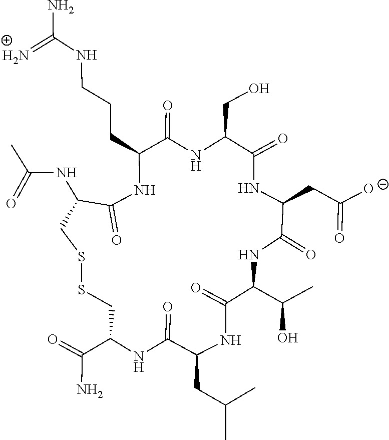 [SMILES: CC(=O)N[C@H]1CSSC[C@@H](C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 6 | 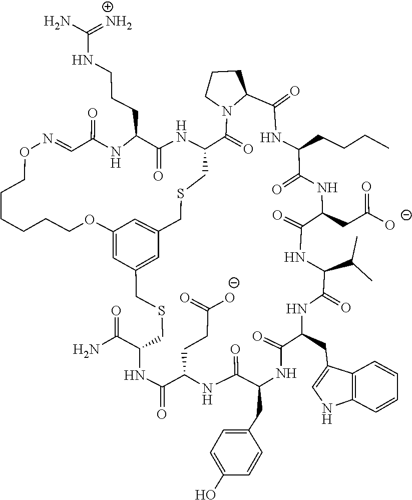 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC(=O)[C@H](C(C)C)NC(=O)[C@H](CC(=O)[O-])NC1=O] |
| 7 | 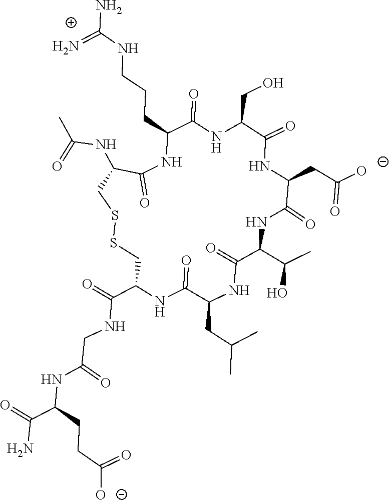 [SMILES: CC(=O)N[C@H]1CSSC[C@@H](C(=O)NCC(=O)N[C@@H](CCC(=O)[O-])C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(=N)N)NC1=O] |
| 8 | 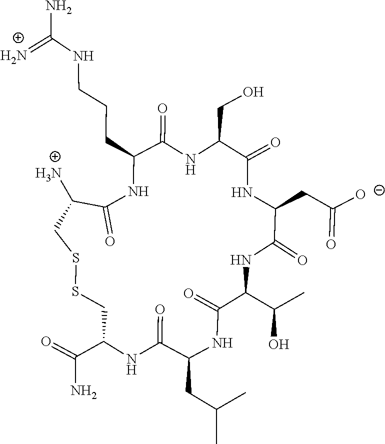 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]([NH3+])CSSC[C@@H](C(N)=O)NC1=O] |
| 9 | 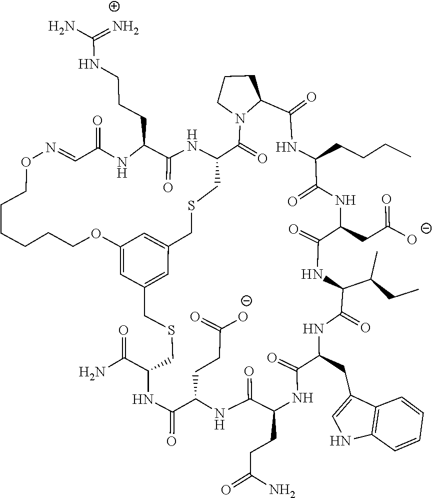 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 10 | 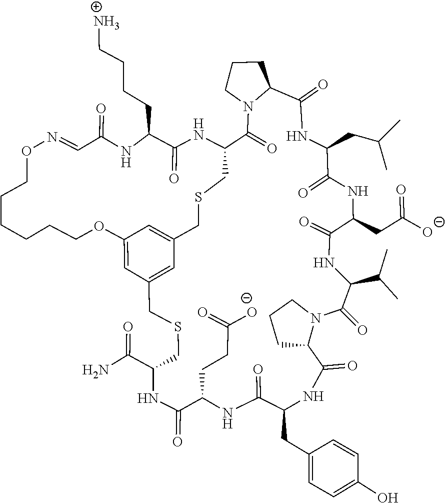 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C(C)C)NC(=O)[C@H](CC(=O)[O-])NC1=O] |
| 11 | 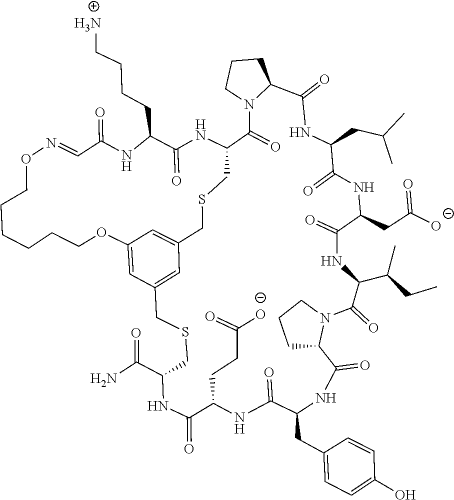 [SMILES: CC[C@H](C)[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 12 | 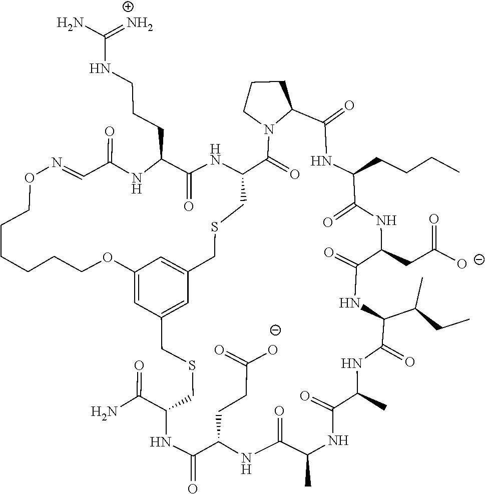 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](C)NC(=O)[C@H](C)NC(=O)[C@H]([C@@H](C)CC)NC(=O)[C@H](CC(=O)[O-])NC1=O] |
| 13 | 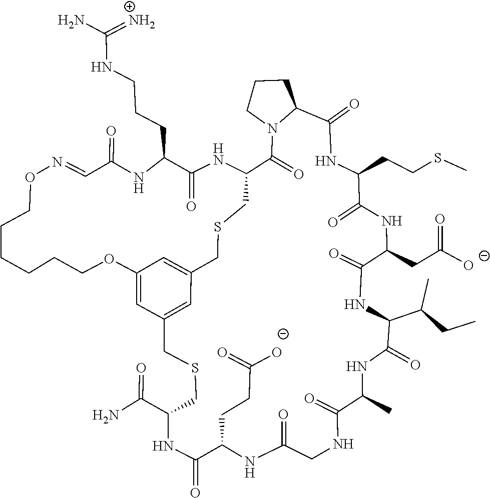 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC1=O] |
| 14 | 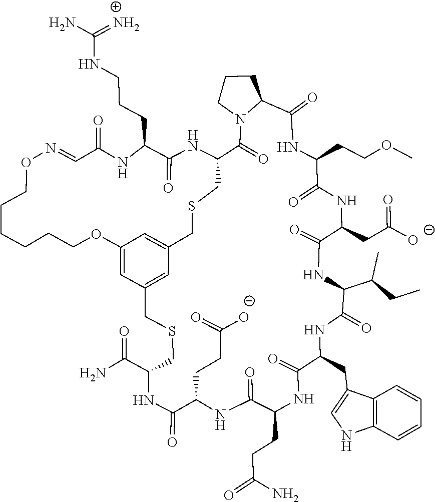 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 15 | 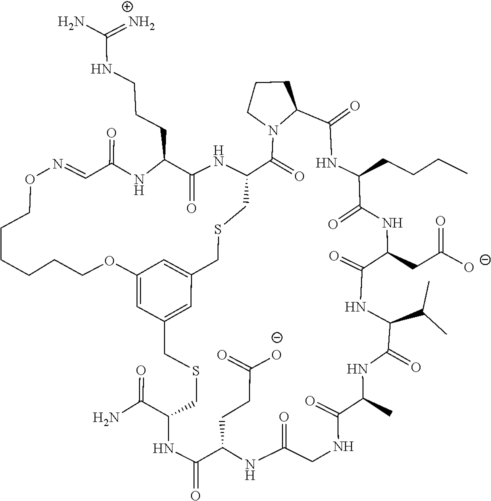 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 16 | 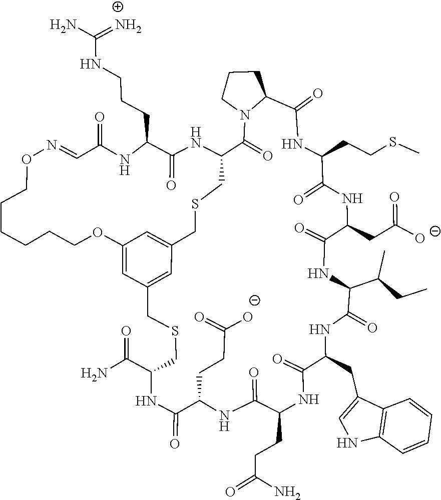 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 17 | 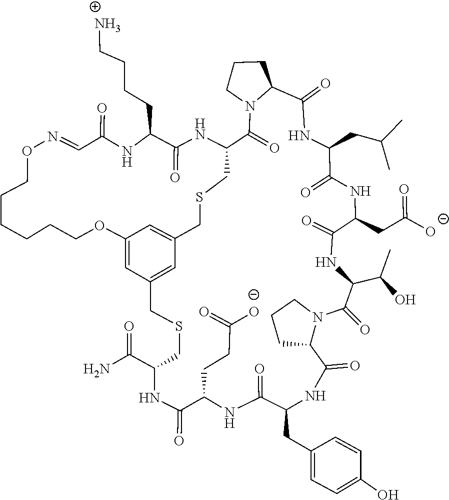 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 18 | 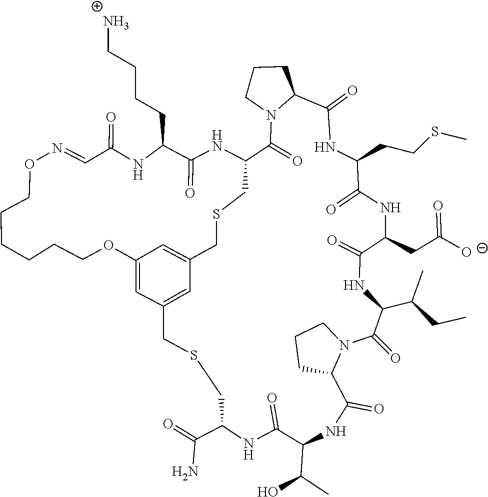 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H]([C@@H](C)O)NC(=O)[C@@H]2CCCN2C1=O] |
| 19 | 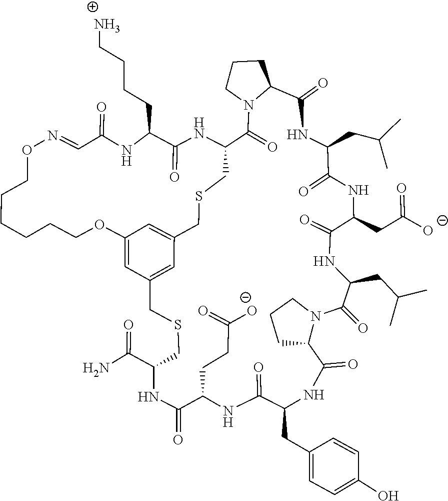 |
| 20 | 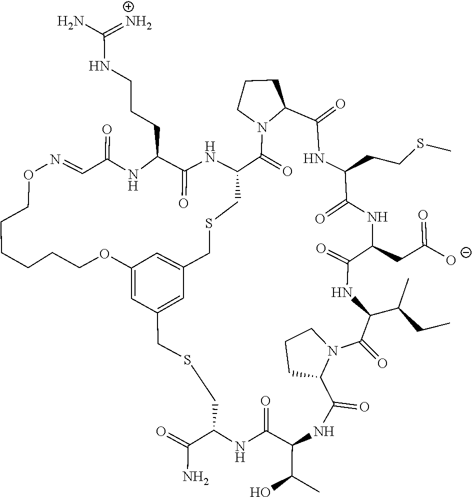 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H]([C@@H](C)O)NC(=O)[C@@H]2CCCN2C1=O] |
| 21 | 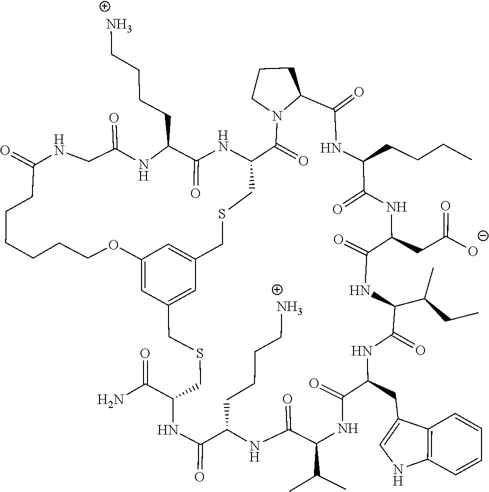 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCC[NH3+])C(=O)N2)CSC[C@@H](C(N)=O)NC(=O)[C@H](CCCC[NH3+])NC(=O)[C@H](C(C)C)NC(=O)[C@H](Cc2c[nH]c3ccccc23)NC(=O)[C@H]([C@@H](C)CC)NC(=O)[C@H](CC(=O)[O-])NC1=O] |
| 22 | 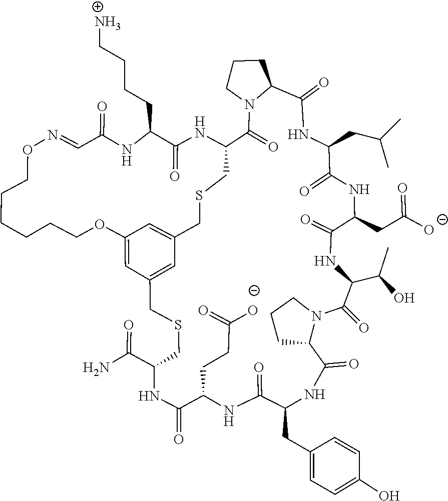 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 23 | 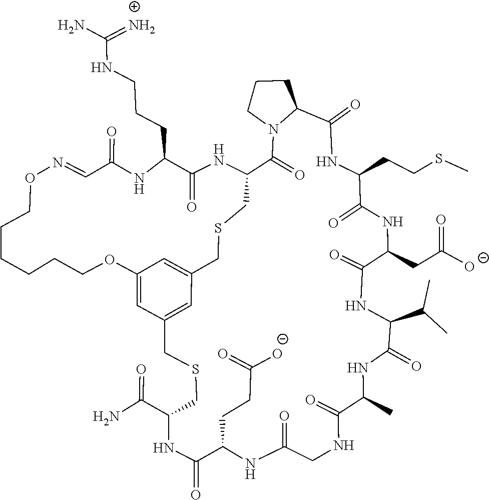 [SMILES: CSCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 24 | 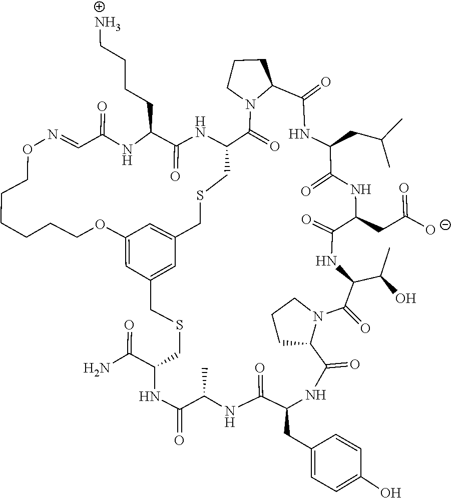 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 25 | 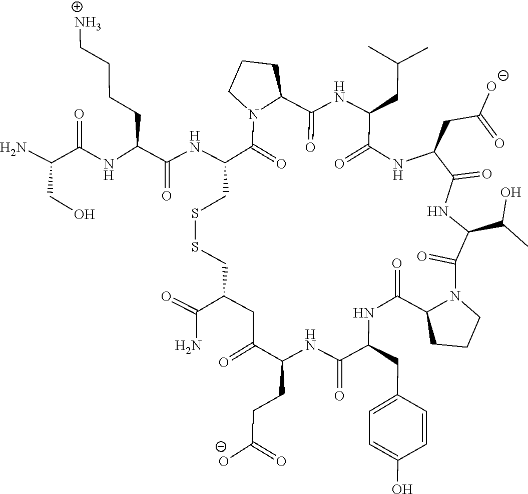 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)CC(=O)[C@H](CCC(=O)[O-])NC(=O)[C@H](Cc2ccc(O)cc2)NC(=O)[C@@H]2CCCN2C(=O)[C@H](C(C)O)NC(=O)[C@H](CC(=O)[O-])NC1=O] |
| 26 | 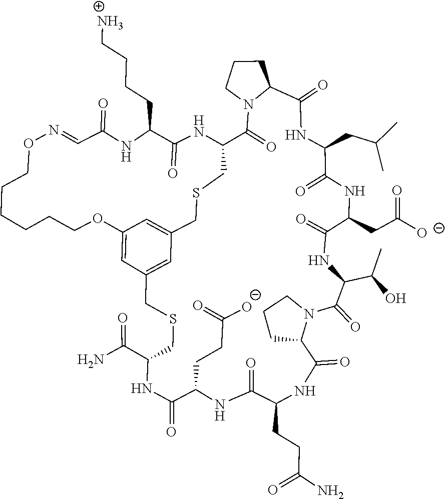 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](CCC(N)=O)NC(=O)[C@@H]2CCCN2C(=O)[C@H]([C@@H](C)O)NC(=O)[C@H](CC(=O)[O-])NC1=O] |
| 27 | 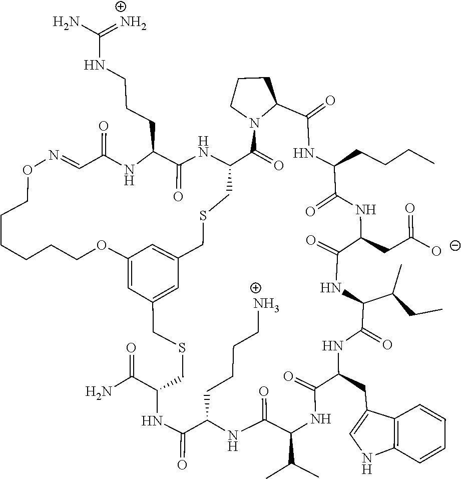 |
| 28 | 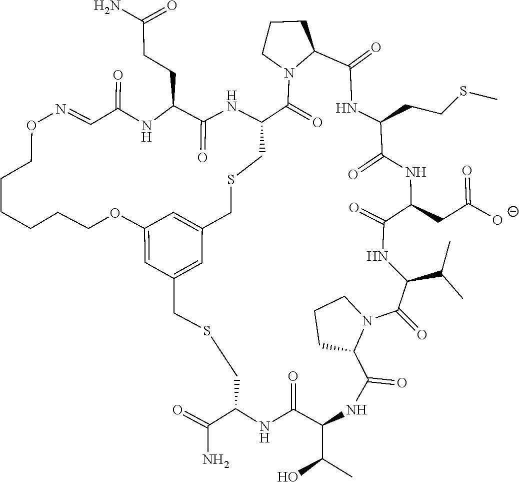 [SMILES: CSCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C(=O)[C@H](C(C)C)NC(=O)[C@H](CC(=O)[O-])NC1=O] |
| 29 | 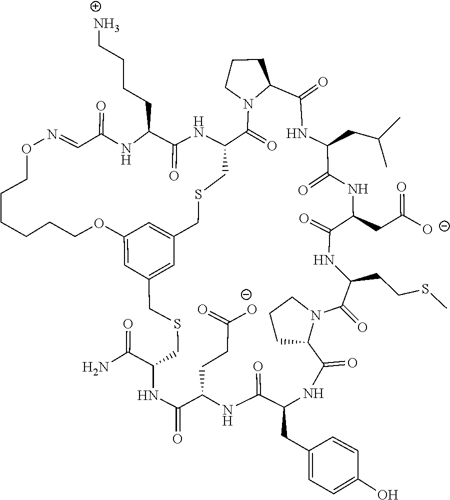 [SMILES: CSCC[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 30 | 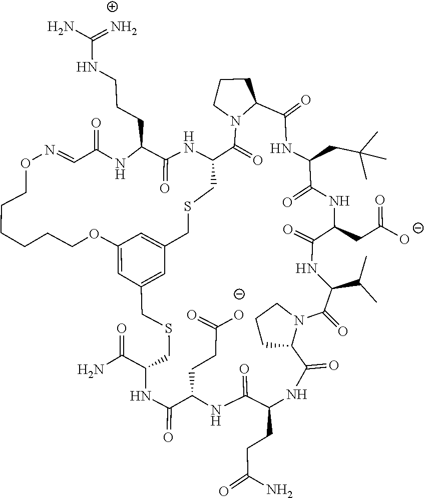 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@@H]2CCCN2C1=O] |
| 31 | 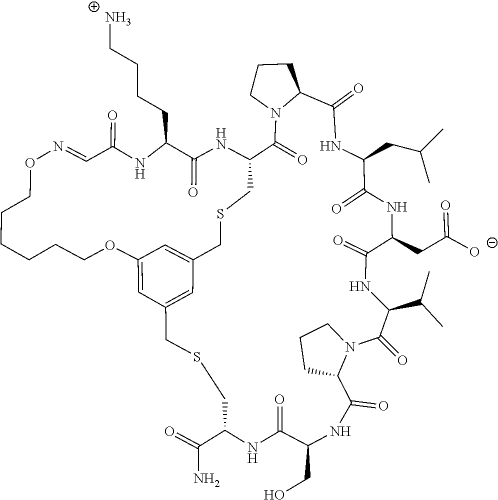 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 32 | 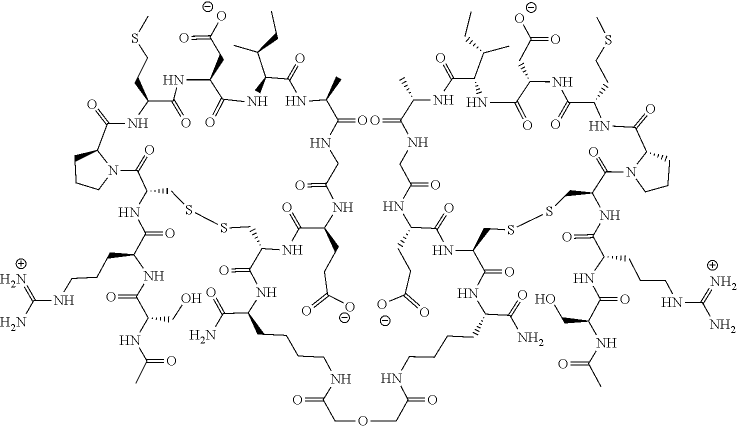 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(=O)N[C@@H](CCCCNC(=O)COCC(=O)NCCCC[C@H](NC(=O)[C@@H]2CSSC[C@H](NC(=O)[C@H](CCCNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)NC(=O)[C@H](CCC(=O)[O-])NC(=O)CNC(=O)[C@H](C)NC1=O] |
| 33 | 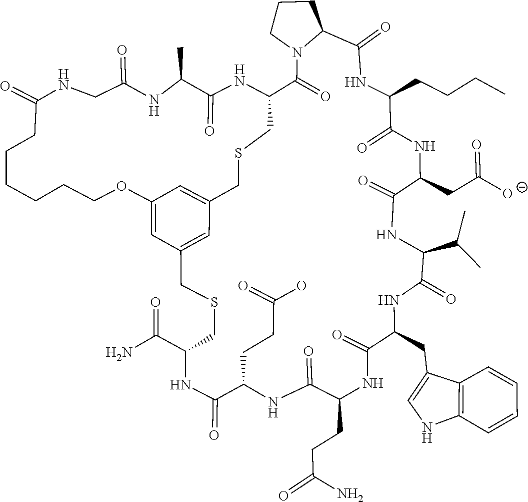 |
| 34 | 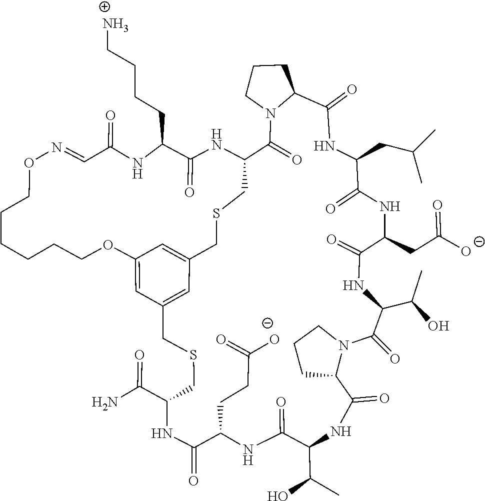 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H]([C@@H](C)O)NC(=O)[C@@H]2CCCN2C(=O)[C@H]([C@@H](C)O)NC(=O)[C@H](CC(=O)[O-])NC1=O] |
| 35 | 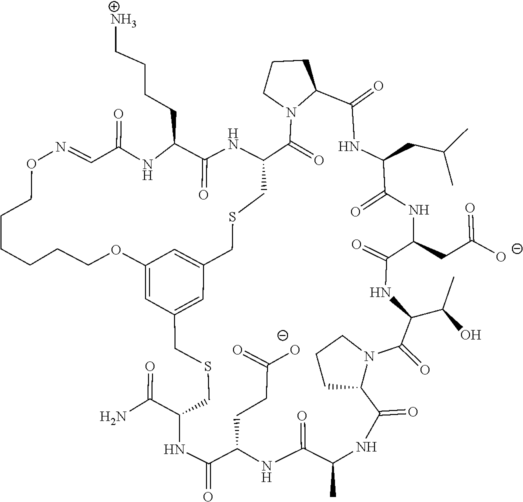 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](C)NC(=O)[C@@H]2CCCN2C(=O)[C@H]([C@@H](C)O)NC(=O)[C@H](CC(=O)[O-])NC1=O] |
| 36 | 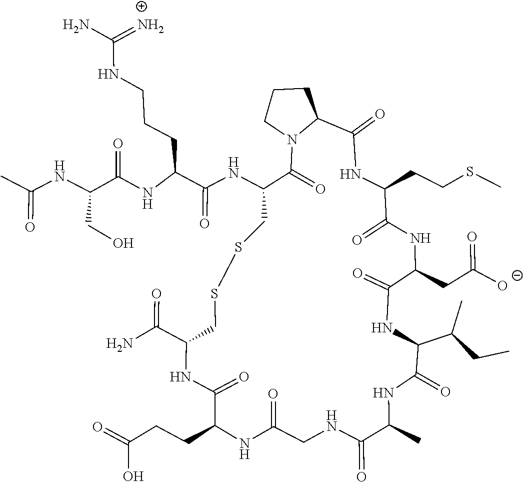 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(N)=O)NC(=O)[C@H](CCC(=O)O)NC(=O)CNC(=O)[C@H](C)NC1=O] |
| 37 | 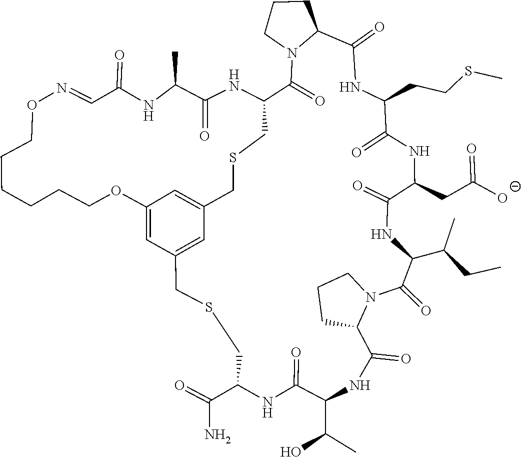 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H]([C@@H](C)O)NC(=O)[C@@H]2CCCN2C1=O] |
| 38 | 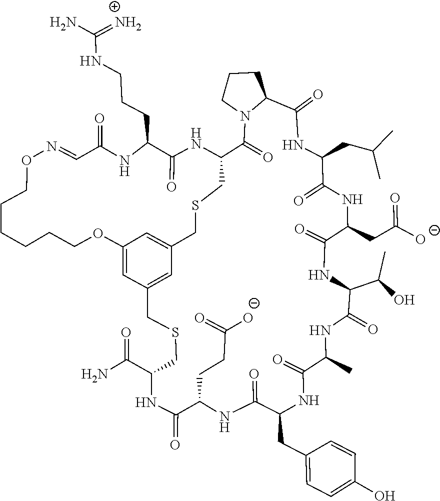 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](C)NC(=O)[C@H]([C@@H](C)O)NC(=O)[C@H](CC(=O)[O-])NC1=O] |
| 39 | 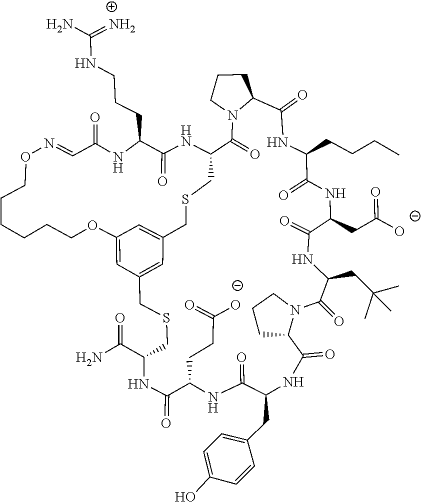 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](CC(C)(C)C)NC(=O)[C@H](CC(=O)[O-])NC1=O] |
| 40 | 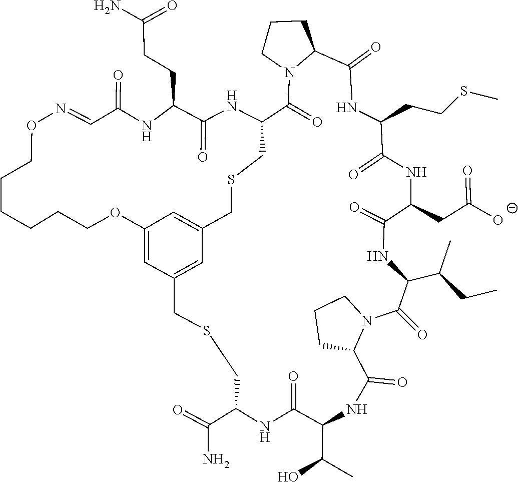 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 41 | 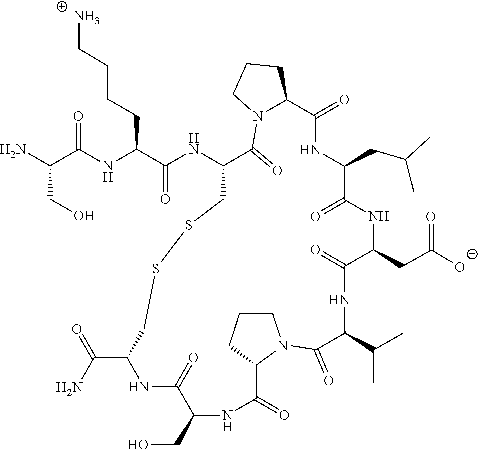 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 42 | 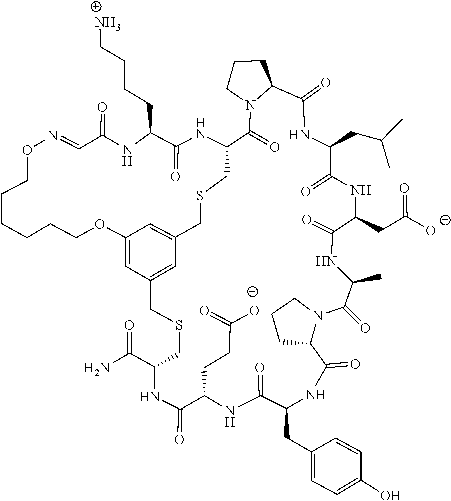 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C)NC(=O)[C@H](CC(=O)[O-])NC1=O] |
| 43 | 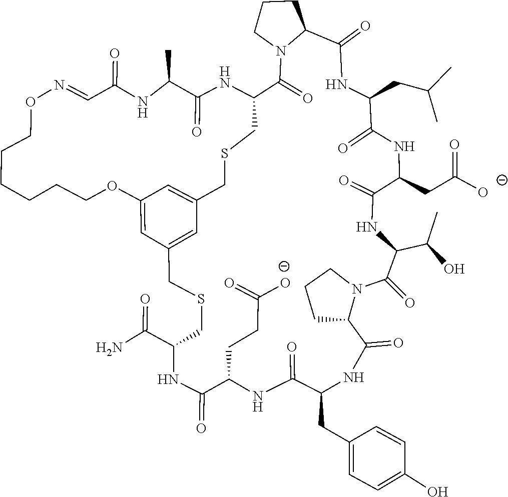 [SMILES: CC(C)C[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 44 | 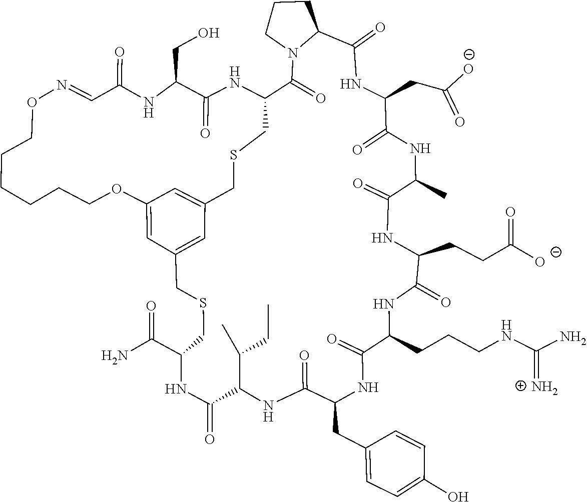 |
| 45 | 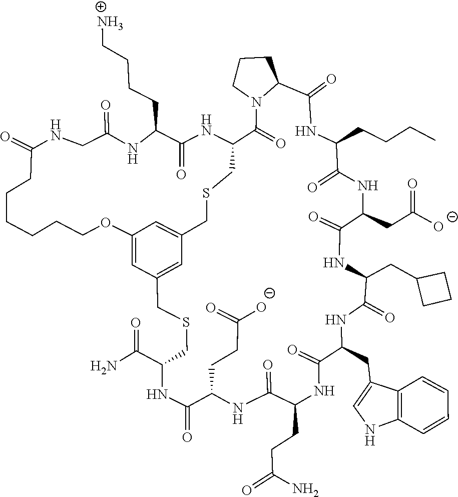 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCC[NH3+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](CC2CCC2)NC(=O)[C@H](CC(=O)[O-])NC1=O] |
| 46 | 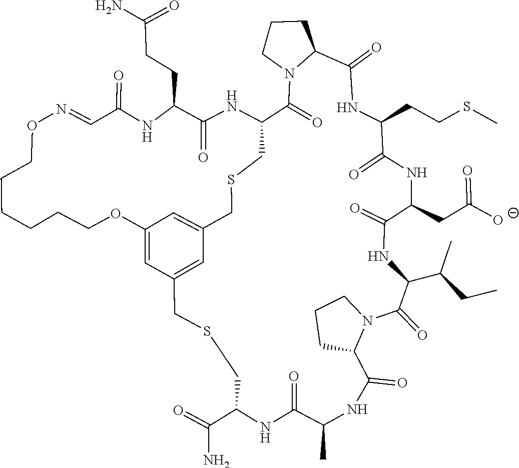 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H](C)NC(=O)[C@@H]2CCCN2C1=O] |
| 47 | 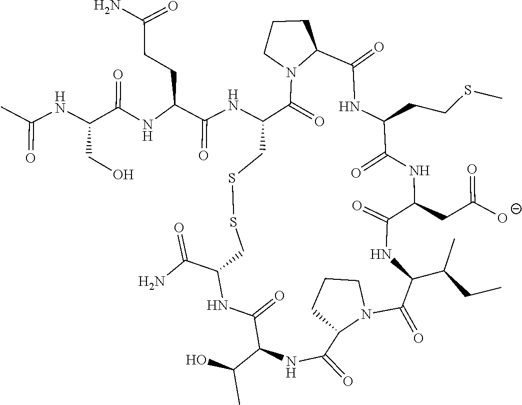 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCC(N)=O)NC(=O)[C@H](CO)NC(C)=O)CSSC[C@@H](C(N)=O)NC(=O)[C@H]([C@@H](C)O)NC(=O)[C@@H]2CCCN2C1=O] |
| 48 | 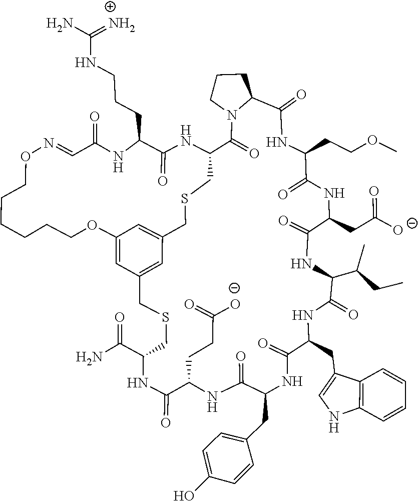 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC1=O] |
| 49 | 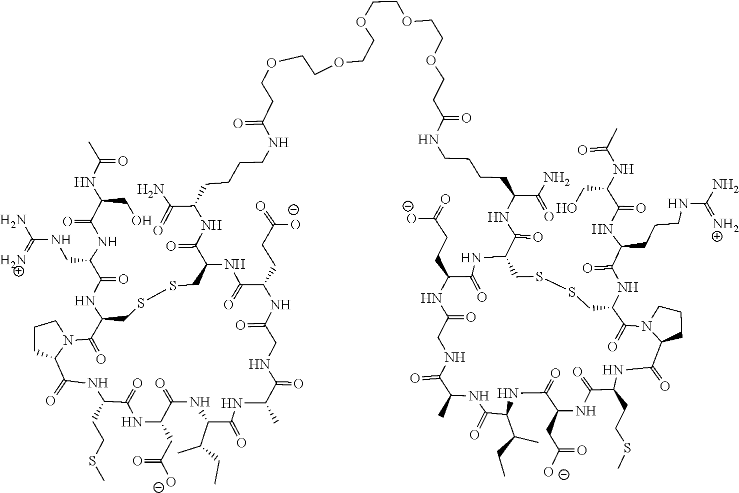 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(=O)N[C@@H](CCCCNC(=O)CCOCCOCCOCCOCCOCCC(=O)NCCCC[C@H](NC(=O)[C@@H]2CSSC[C@H](NC(=O)[C@H](CNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)NC(=O)[C@H](CCC(=O)[O-])NC(=O)CNC(=O)[C@H](C)NC1=O] |
| 50 | 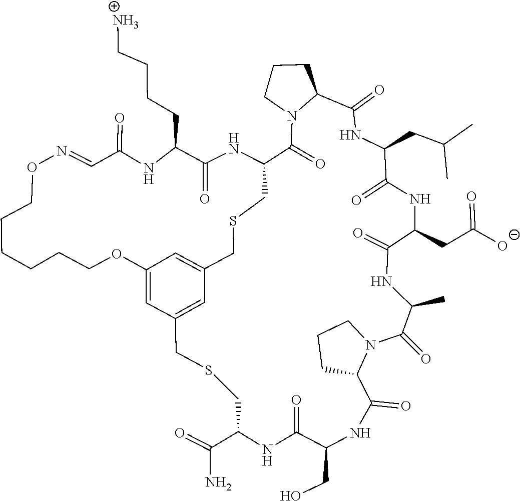 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C)NC(=O)[C@H](CC(=O)[O-])NC1=O] |
| 51 | 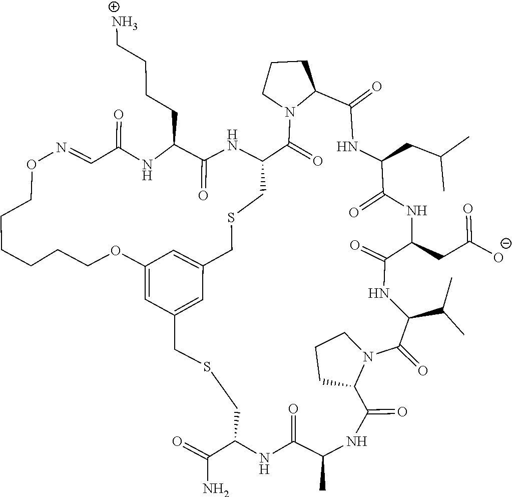 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@@H]2CCCN2C(=O)[C@H](C(C)C)NC(=O)[C@H](CC(=O)[O-])NC1=O] |
| 52 | 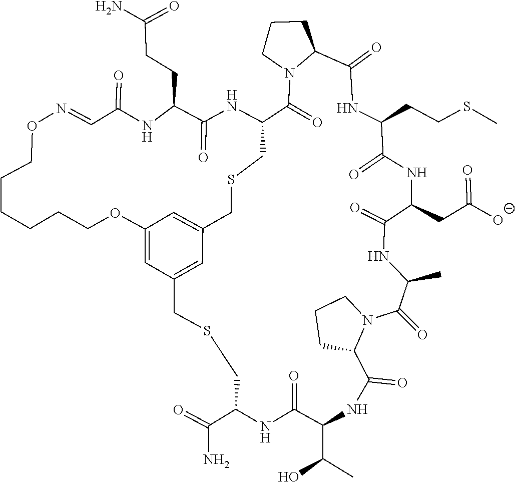 [SMILES: CSCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C(=O)[C@H](C)NC(=O)[C@H](CC(=O)[O-])NC1=O] |
| 53 | 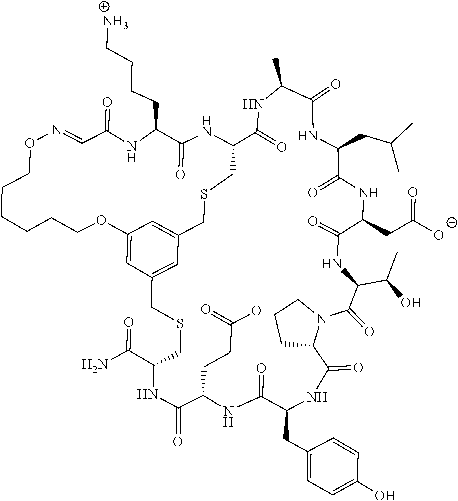 |
| 54 | 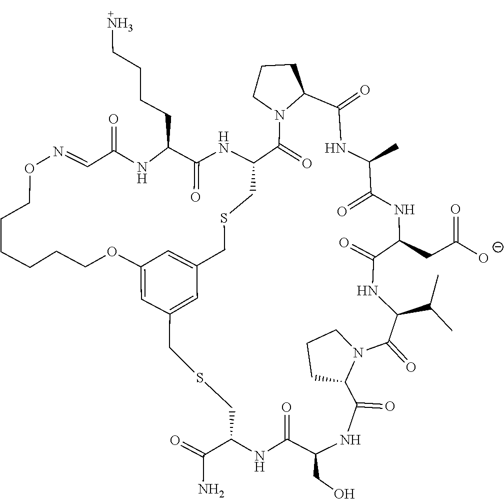 [SMILES: CC(C)[C@@H]1NC(=O)[C@H](CC(=O)[O-])NC(=O)[C@H](C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C1=O] |
| 55 | 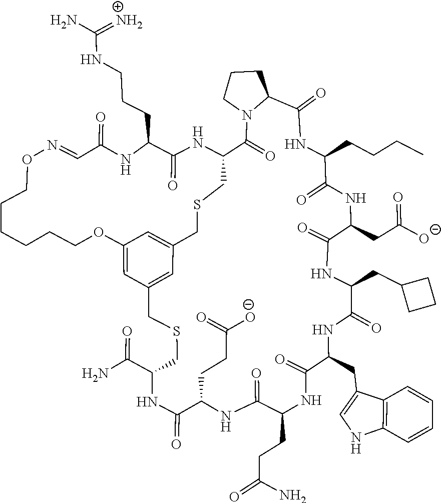 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](CC2CCC2)NC(=O)[C@H](CC(=O)[O-])NC1=O] |
| 56 | 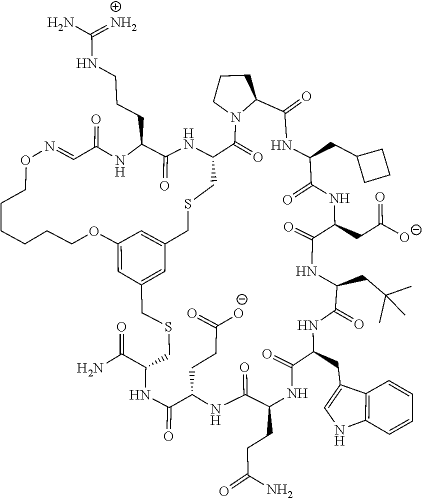 [SMILES: CC(C)(C)C[C@@H]1NC(=O)[C@H](CC(=O)[O-])NC(=O)[C@H](CC2CCC2)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 57 | 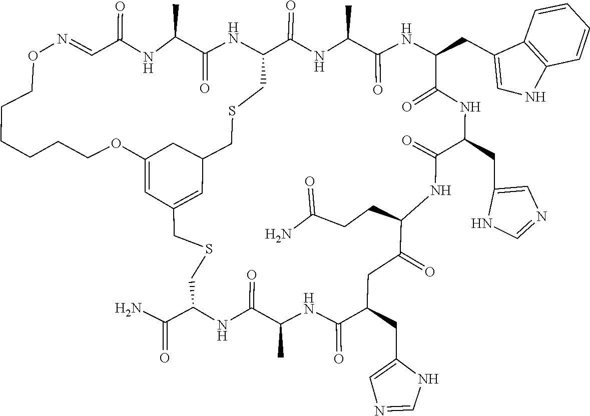 [SMILES: C[C@@H]1NC(=O)/C=N/OCCCCCCOC2=CC3=CC(CSC[C@H](NC1=O)C(=O)N[C@@H](C)C(=O)N[C@@H](Cc1c[nH]c4ccccc14)C(=O)N[C@@H](Cc1cnc[nH]1)C(=O)N[C@H](CCC(N)=O)C(=O)C[C@@H](Cc1cnc[nH]1)C(=O)N[C@@H](C)C(=O)N[C@H](C(N)=O)CSC3)C2] |
| 58 | 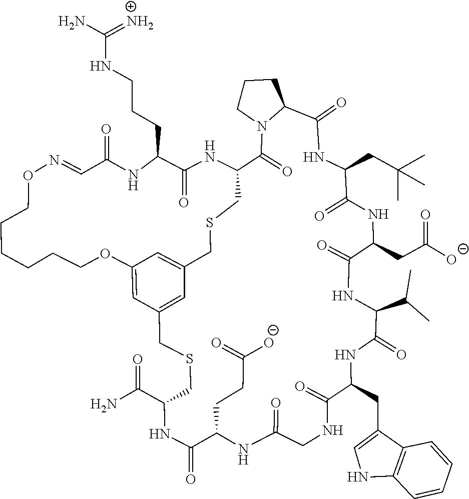 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](Cc2c[nH]c3ccccc23)NC1=O] |
| 59 | 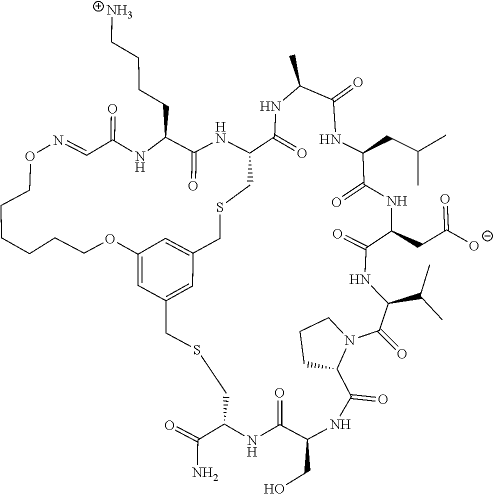 [SMILES: CC(C)C[C@@H]1NC(=O)[C@H](C)NC(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 60 | 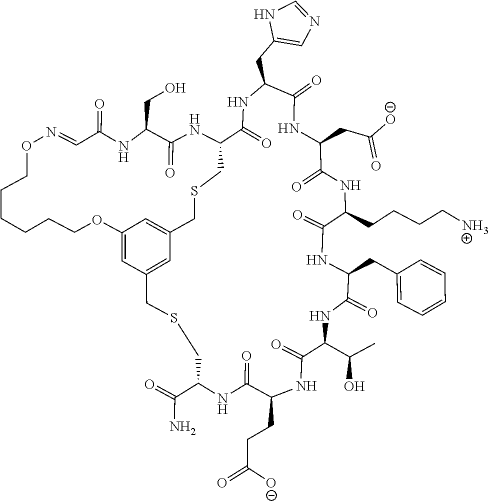 [SMILES: C[C@@H](O)[C@@H]1NC(=O)[C@H](Cc2ccccc2)NC(=O)[C@H](CCCC[NH3+])NC(=O)[C@H](CC(=O)[O-])NC(=O)[C@H](Cc2cnc[nH]2)NC(=O)[C@@H]2CSCc3cc(cc(c3)OCCCCCCO/N=C/C(=O)N[C@@H](CO)C(=O)N2)CSC[C@@H](C(N)=O)NC(=O)[C@H](CCC(=O)[O-])NC1=O] |
| 61 | 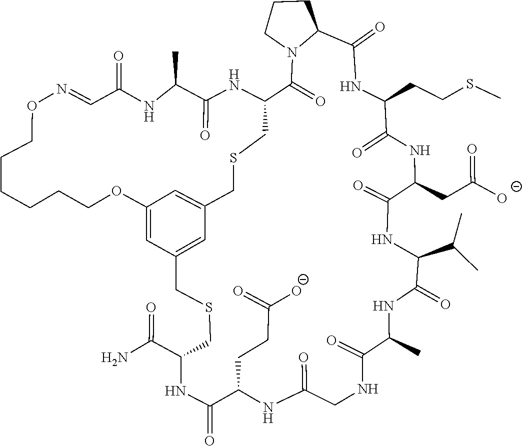 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 62 | 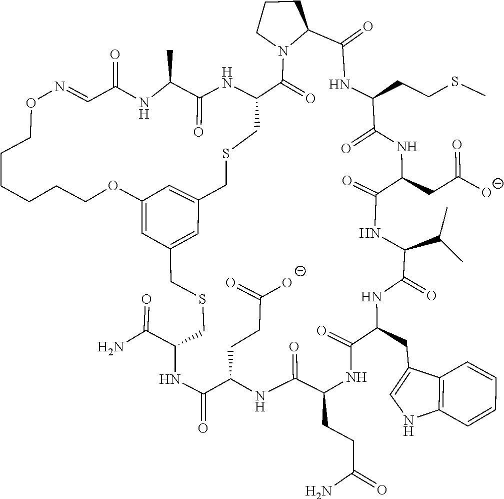 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 63 | 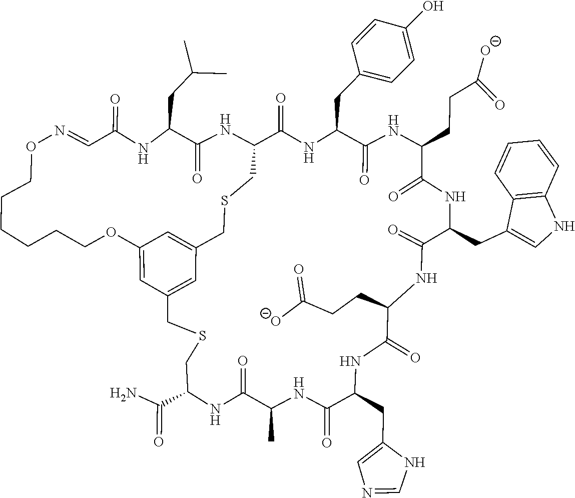 [SMILES: CC(C)C[C@@H]1NC(=O)/C=N/OCCCCCCOc2cc3cc(c2)CSC[C@H](NC1=O)C(=O)N[C@@H](Cc1ccc(O)cc1)C(=O)N[C@@H](CCC(=O)[O-])C(=O)N[C@@H](Cc1c[nH]c2ccccc12)C(=O)N[C@H](CCC(=O)[O-])C(=O)N[C@@H](Cc1cnc[nH]1)C(=O)N[C@@H](C)C(=O)N[C@H](C(N)=O)CSC3] |

Another embodiment described herein is a compound selected from the group consisting of:

| No. | Structure |
|---|---|
| 1 | 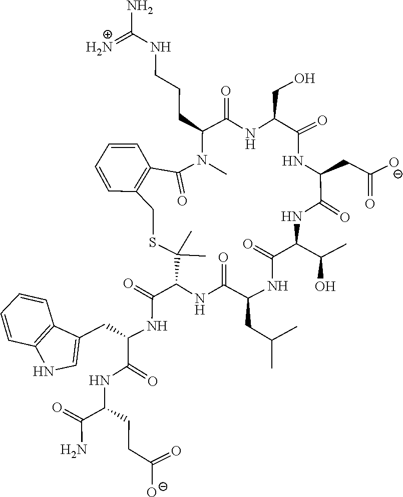 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)c2ccccc2CSC(C)(C)[C@@H](C(=O)N[C@@H](Cc2c[nH]c3ccccc23)C(=O)N[C@H](CCC(=O)[O-])C(N)=O)NC1=O] |
| 2 | 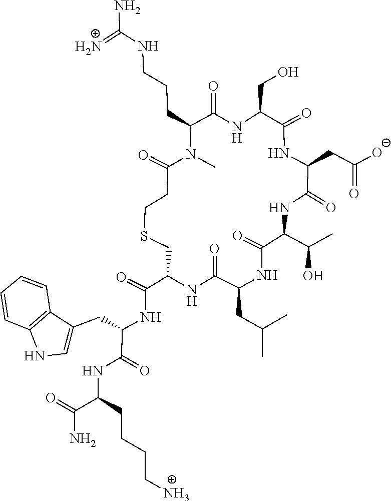 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)CCSC[C@@H](C(=O)N[C@@H](Cc2c[nH]c3ccccc23)C(=O)N[C@@H](CCCC[NH3+])C(N)=O)NC1=O] |
| 3 | 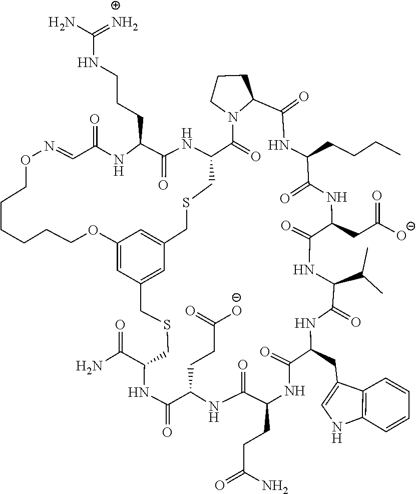 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 4 | 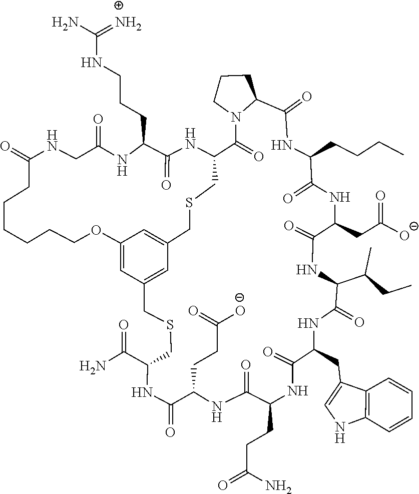 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 5 | 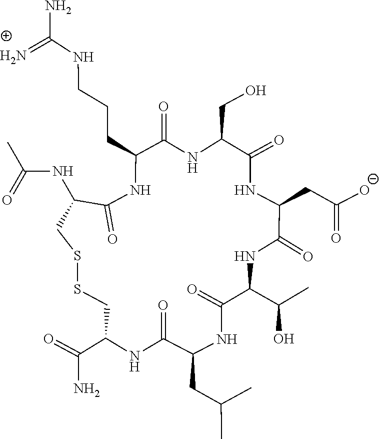 [SMILES: CC(=O)N[C@H]1CSSC[C@@H](C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 6 | 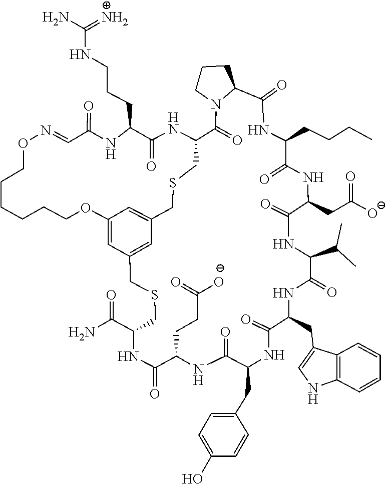 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC(=O)[C@H](C(C)C)NC(=O)[C@H](CC(=O)[O-])NC1=O] |
| 7 | 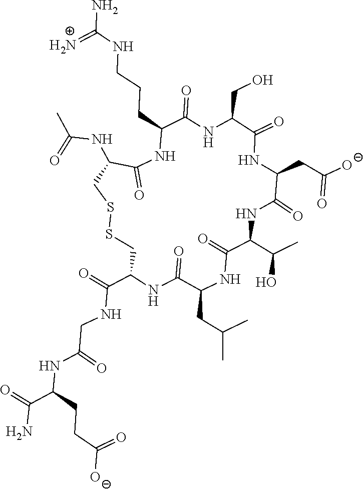 [SMILES: CC(=O)N[C@H]1CSSC[C@@H](C(=O)NCC(=O)N[C@@H](CCC(=O)[O-])C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 8 | 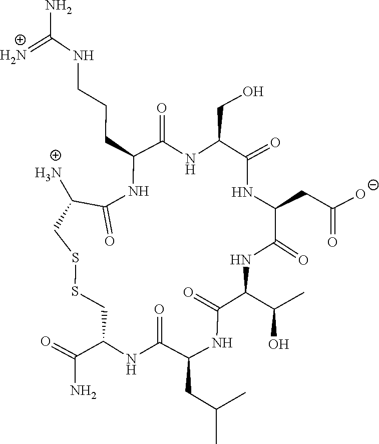 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]([NH3+])CSSC[C@@H](C(N)=O)NC1=O] |
| 9 | 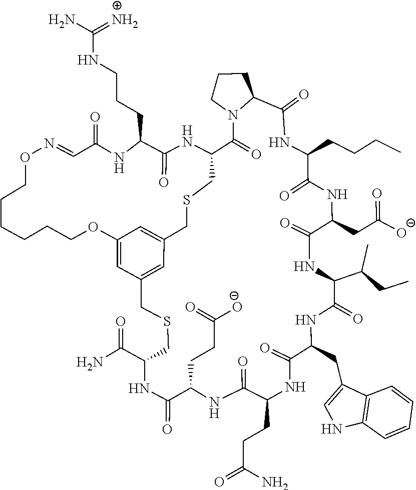 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 10 | 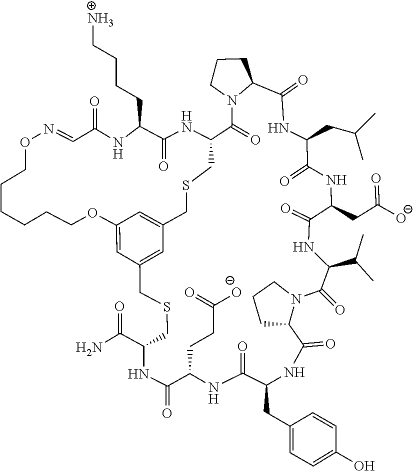 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C(C)C)NC(=O)[C@H](CC(=O)[O-])NC1=O] |
| 11 | 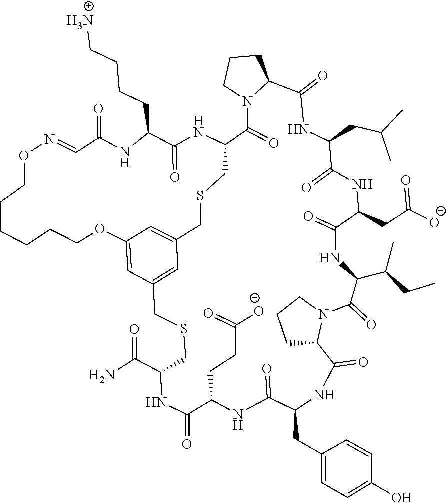 [SMILES: CC[C@H](C)[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 12 | 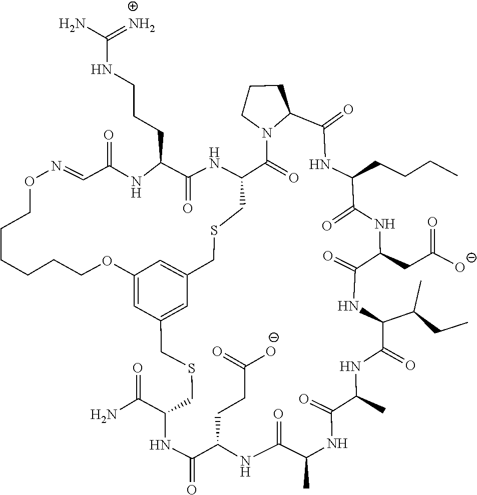 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](C)NC(=O)[C@H](C)NC(=O)[C@H]([C@@H](C)CC)NC(=O)[C@H](CC(=O)[O-])NC1=O] |
| 13 | 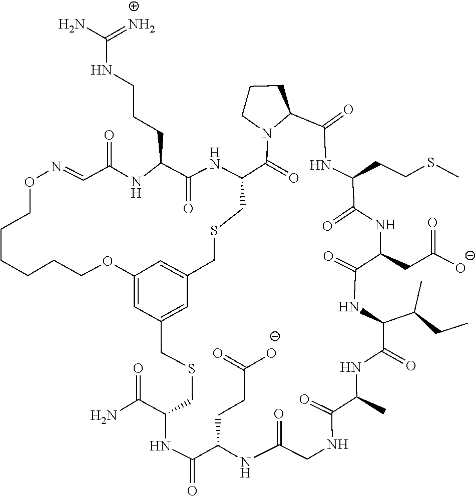 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC1=O] |
| 14 | 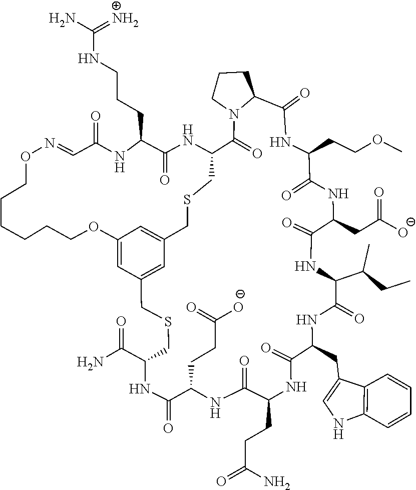 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 15 | 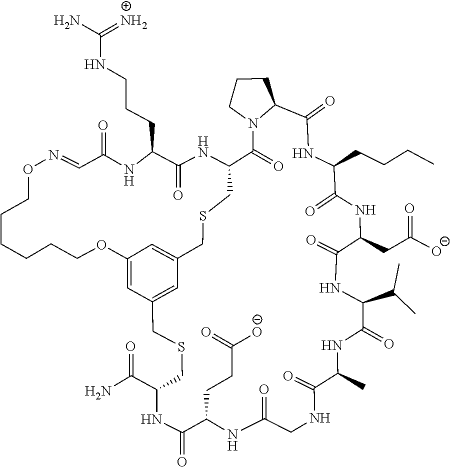 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 16 | 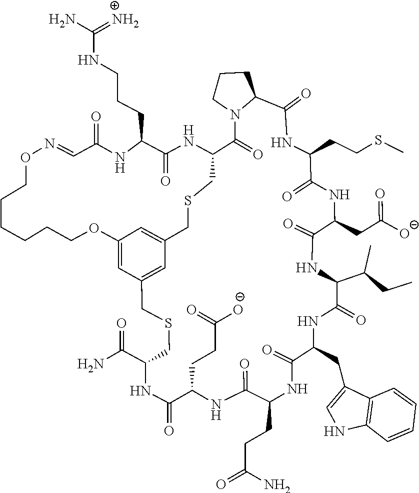 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 17 | 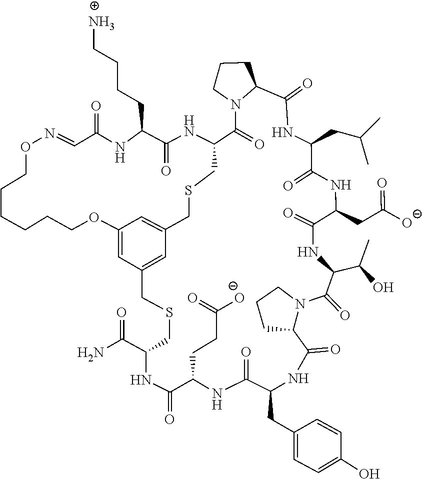 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 18 | 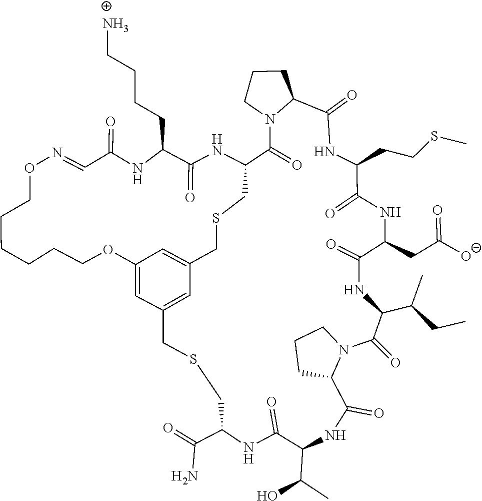 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H]([C@@H](C)O)NC(=O)[C@@H]2CCCN2C1=O] |
| 19 | 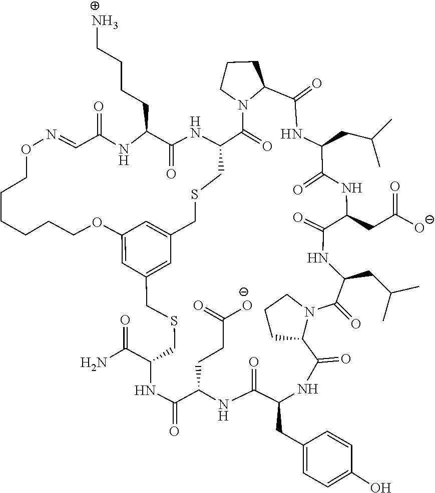 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](CC(C)C)NC(=O)[C@H](CC(=O)[O-])NC1=O] |
| 20 | 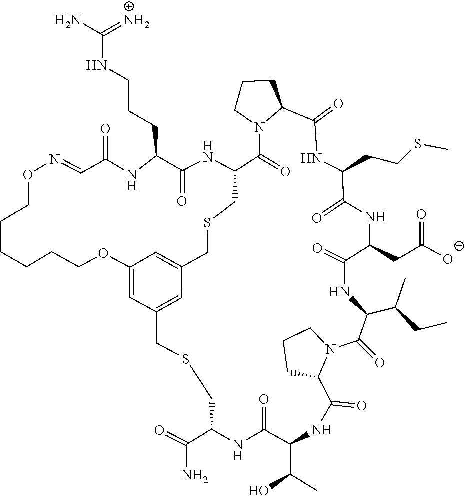 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H]([C@@H](C)O)NC(=O)[C@@H]2CCCN2C1=O] |

In one aspect is a pharmaceutical composition comprising any of the compounds described herein or a pharmaceutically acceptable salt thereof.

Another aspect described herein is a method for treating a disease or condition associated with biological function of α4β7 comprising administering to a subject in need thereof an effective amount of the pharmaceutical composition described herein.

Another aspect described herein is a method for treating a disease or condition associated with biological function of MAdCAM comprising administering to a subject in need thereof an effective amount of the pharmaceutical composition described herein.

Another aspect described herein is a method of treating disease comprising administering to a subject in need thereof an effective amount of the pharmaceutical composition described herein.

In one aspect the disease comprises inflammatory bowel disease, ulcerative colitis, Crohn's disease, Celiac disease, colitis, diverticulitis, eosinophilic gastroenteritis, gastrointestinal cancer, autoimmune hepatitis, pancreatitis, encephalomyelitis, psoriasis and other inflammatory dermatoses such as dermatitis, eczema, atopic dermatitis, allergic contact dermatitis, urticarial, and pruritus, autoimmune diseases, such as fibromyalgia, scleroderma, ankylosing spondylitis, juvenile rheumatoid arthritis (RA), Still's disease, polyarticular juvenile RA, pauciarticular juvenile RA, polymyalgia rheumatica, RA, psoriatic arthritis, osteoarthritis, polyarticular arthritis, multiple sclerosis, lupus, systemic lupus erythematosus, type I diabetes (T1DM), type II diabetes (T2DM), glomerulonephritis, graft-v-host disease (including both acute and chronic), and HIV infection.

In one aspect described herein is a compound for use for treating a disease or condition associated with biological function of α4β7 comprising administering to a subject in need thereof an effective amount of any of the compounds described herein.

In one aspect described herein is a compound for use for treating a disease or condition associated with biological function of MAdCAM comprising administering to a subject in need thereof an effective amount of any of the compounds described herein. In one aspect described herein is a compound for use for treating disease comprising administering to a subject in need thereof an effective amount of any of the compounds described herein.

In one aspect, the disease comprises inflammatory bowel disease, ulcerative colitis, Crohn's disease, Celiac disease, colitis, diverticulitis, eosinophilic gastroenteritis, gastrointestinal cancer, autoimmune hepatitis, pancreatitis, encephalomyelitis, psoriasis and other inflammatory dermatoses such as dermatitis, eczema, atopic dermatitis, allergic contact dermatitis, urticarial, and pruritus, autoimmune diseases, such as fibromyalgia, scleroderma, ankylosing spondylitis, juvenile rheumatoid arthritis (RA), Still's disease, polyarticular juvenile RA, pauciarticular juvenile RA, polymyalgia rheumatica, RA, psoriatic arthritis, osteoarthritis, polyarticular arthritis, multiple sclerosis, lupus, systemic lupus erythematosus, type I diabetes (T1DM), type II diabetes (T2DM), glomerulonephritis, graft-v-host disease (including both acute and chronic), and HIV infection.

Another aspect described herein is a use of any of the compounds described herein for the treatment of a disease or condition associated with biological function of α4β7.

Another aspect described herein is a use of any of the compounds described herein for the treatment of a disease or condition associated with biological function of MAdCAM.

Another aspect described herein is a use of any of the compounds described herein for treatment of a disease comprising administering to a subject in need thereof an effective amount of any of the compounds described herein.

In one aspect the disease comprises inflammatory bowel disease, ulcerative colitis, Crohn's disease, Celiac disease, colitis, diverticulitis, eosinophilic gastroenteritis, gastrointestinal cancer, autoimmune hepatitis, pancreatitis, encephalomyelitis, psoriasis and other inflammatory dermatoses such as dermatitis, eczema, atopic dermatitis, allergic contact dermatitis, urticarial, and pruritus, autoimmune diseases, such as fibromyalgia, scleroderma, ankylosing spondylitis, juvenile rheumatoid arthritis (RA), Still's disease, polyarticular juvenile RA, pauciarticular juvenile RA, polymyalgia rheumatica, RA, psoriatic arthritis, osteoarthritis, polyarticular arthritis, multiple sclerosis, lupus, systemic lupus erythematosus, type I diabetes (T1DM), type II diabetes (T2DM), glomerulonephritis, graft-v-host disease (including both acute and chronic), and HIV infection.

### BRIEF DESCRIPTION OF THE DRAWINGS

FIG. 1: describes a peptide scaffold as described herein wherein the bicyclic structure is obtained using various linkers that ligate to specific amino acid residues within the peptide scaffold.

FIG. 2A: describes exemplary linkers including two-fold symmetric linchin (TSL) linkers (TSL-1, TSL-3, TSL-6, and TSLC-7) all having thiol-reactive chlorobenzyl functionalities and Pa, Bz, polyethylene glycol5 (PEG5), chloroacetic acid, and 2,2′oxydiacetic acid (DIG)

FIG. 2B: describes modified amino acids present in the representative compounds of Table 3.

FIG. 3: describes an exemplary compound comprising the bicyclic scaffold of peptides according to Formula (I), Formula (III) or Formula (IV) using the TSL-6 linker.

FIG. 4: describes results from the Competition Binding Assay of Example 1.

FIG. 5: describes heat maps of positional scans generated for the compounds of the present disclosure comprising peptide sequences of SEQ ID NOs. 16, 23, 131, 61, 62 and 68.

FIG. 6: describes the compounds of the present disclosure.

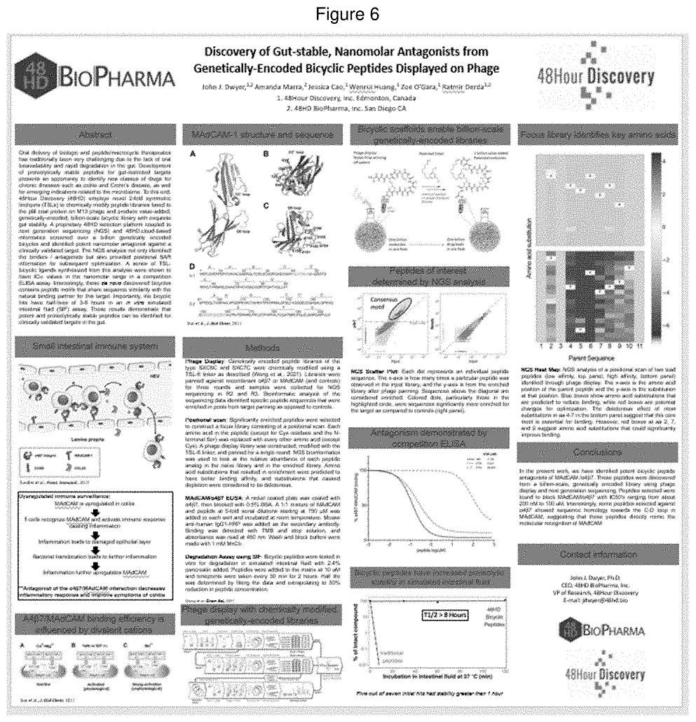

### DETAILED DESCRIPTION OF THE INVENTION

The present disclosure related to novel compounds for the modulation of α4β7 and MAdCAM, and methods of treating disease or conditions associated with their biological function, including inflammatory bowel disease, ulcerative colitis and Crohn's disease.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 15%, 10%, 5%, or 1%.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be detected and/or further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a therapeutically effective amount.

As used herein the term “amino acid” or “any amino acid” refers to all naturally occurring amino acids, non-naturally occurring amino acids, chemically modified amino acids, synthetic amino acids and the L- and/or D-isomeric forms of such amino acids. Furthermore, amino acids as described herein may be referred to by their full name, three letter abbreviation, or single letter abbreviation, all interchangeably, and as described in Table 1:

TABLE 1
Amino Acid Terminology

| Amino Acid | 3-Letter | 1-Letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

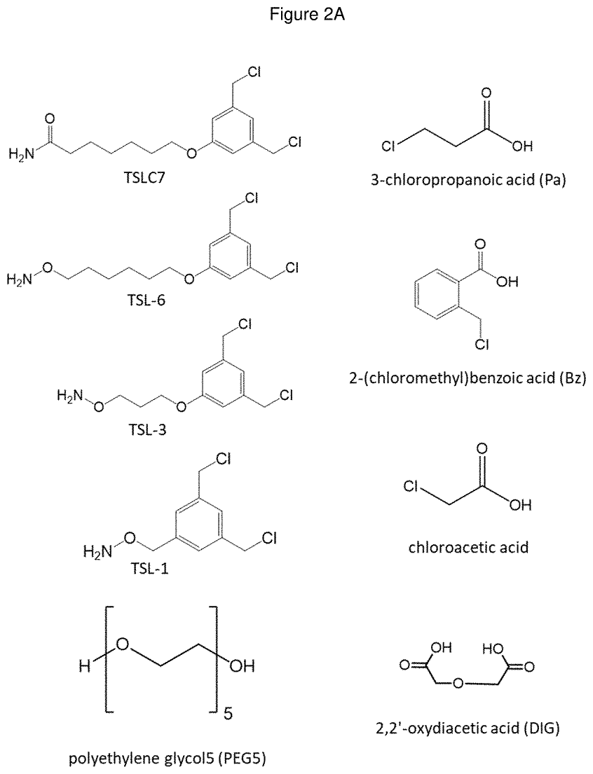

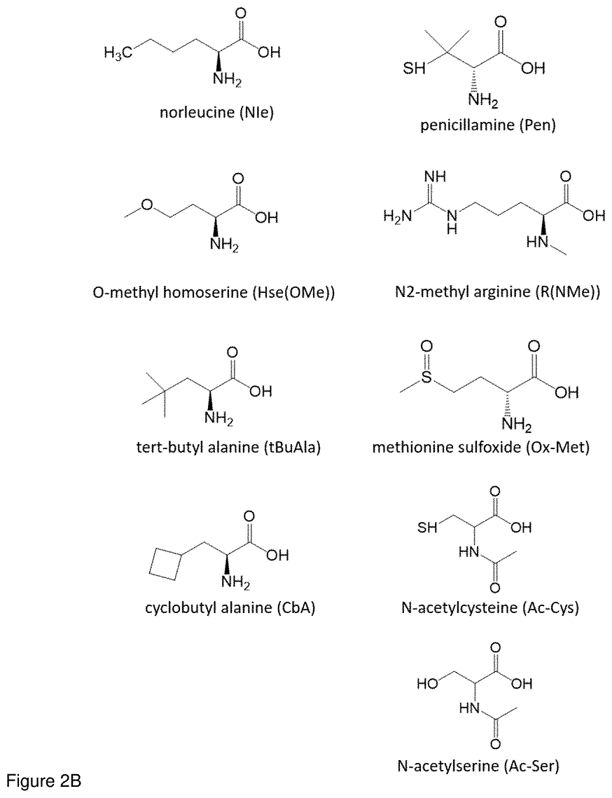

Additional modified amino acids, include but are not limited to, those listed in Table 1 b and the corresponding structures are shown in FIG. 2B.

TABLE 1b
Additional Amino Acids

| Amino Acid | Abbreviation |
|---|---|
| Norleucine | Nle |
| O-methyl homoserine | Hse(OMe) |
| tert-butyl alanine | tBuAla |
| Cyclobutyl alanine | CbA |
| penicillamine | Pen |
| N2-methyl arginine | R(NMe) |
| Methionine sulfoxide | Ox-Met |
| N-acetylserine | Ac-Ser |
| N-acetylcysteine | Ac-Cys |

As used herein, the terms “individual” and “subject” are often used interchangeably and refer to any animal that may be treated with the methods disclosed herein. Suitable subjects (e.g., patients) include laboratory animals (such as mouse, rat, rabbit, or guinea pig), farm animals, and domestic animals or pets (such as a cat or dog). Non-human primates and human patients are included. In one embodiment, subjects may include human patients that have been diagnosed with an inflammatory bowel disease, ulcerative colitis or Crohn's disease. As used herein, the term “patient” refers to a subject that may receive a treatment of a disease or condition.

As used herein, the term “peptide” refers to chains of between 2 to 50 amino acids of any sequence, linked together by peptide bonds that form a peptide scaffold of the compounds described herein. The term “N-terminus” or “amino terminus” refers to the terminal N(R^{g})₂ (where each R^{g} is H or alkyl) end of a peptide and not part of a peptide bond. The “C-terminus” or “carboxy terminus” refers to the terminal end COOR^{g} (where R^{g} is H or alkyl) of a peptide and not part of a peptide bond. All linear peptides are written according to the standard convention of the N-terminal amino acid on the left and the C-terminal amino acid on the right. Furthermore, the peptide scaffolds contemplated herein may be monomeric or multi-meric.

As used herein “pharmaceutically acceptable salt” refers to any salt of a compound disclosed herein which retains its biological properties and which is not toxic or otherwise undesirable for veterinary, or pharmaceutical use. Such salts may be derived from a variety of organic and inorganic counter-ions known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2 naphthalenesulfonic, 4-toluenesulfonic, camphoric, camphorsulfonic, 4 methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only, salts of non-toxic organic or inorganic acids, such as halides, such as, chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

As used herein, “treatment”, “treat”, and “treating” refer to reversing, alleviating, mitigating, or slowing the progression of, or inhibiting the progress of, a disorder or disease or symptoms associated with such disorder or disease, and as described in more detail herein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure describes novel compounds with integrin modulation capability, specifically for modulating the activity of integrin α437 and/or MAdCAM, both of which together are responsible for T-cell homing to gut-associated tissues. The compounds described herein comprise a peptide scaffold having intramolecular covalent bonds and exhibiting a cyclic or bicyclic topology found to be more stable to proteolysis and other forms of degradation than linear peptides. Wong et al., 2021. These cyclic or bicyclic compounds are also known to exhibit stronger binding interactions with protein targets. Without being bound by any theory, it is believed that the increase in interaction is due to a reduced conformational penalty of the constrained cyclic or bicyclic system compared to a linear system. Thus, the compounds of the present disclosure comprise a peptide scaffold with cyclic or bicyclic topology resulting in modulation α4β7 and/or MAdCAM.

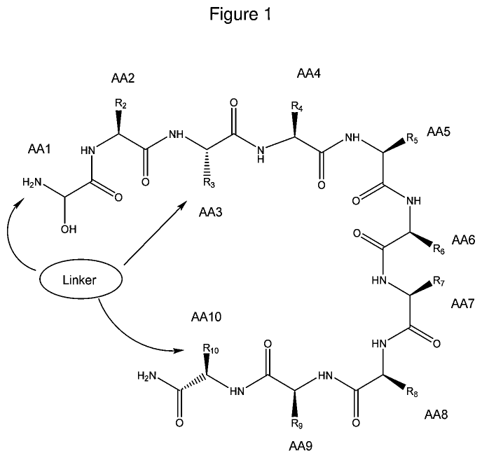

FIG. 1 describes an example of a peptide scaffold for any of the compounds described herein wherein the bicyclic structure is obtained using various linkers that ligate to specific amino acid residues within the peptide scaffold; for example, linkers may ligate at amino acid 1 (AA1), amino acid 3 (AA3) or amino acid 10 (AA10). However, linkers covalently bound to other amino acid residues are also contemplated herein, as well as bicyclic structures formed by a combination of sulfide and disulfide bonds. Thus, in one embodiment described herein is a compound comprising a peptide scaffold and one or more of a linker and a sulfide or disulfide bond, wherein the linker, sulfide, or disulfide bond is covalently bonded to the peptide scaffold.

The compounds of the present disclosure may comprise peptide scaffolds in which the scaffold is between about 2-50 amino acids in length. Peptide scaffolds may also comprise about 1-25 amino acids, about 1-20 amino acids, about 1-15 amino acids, about 1-10 amino acids, or about 1-5 amino acids. These peptide scaffolds may include naturally occurring amino acids, non-naturally occurring amino acids, chemically modified amino acids, synthetic amino acids, or the L- and/or D-isomeric forms of such amino acids. Furthermore, the sequences of the peptide scaffolds disclosed herein confer specificity for the target α4β7 and/or MAdCAM.

Thus, in one aspect described herein is a compound comprising a peptide scaffold having a sequence of Formula (I) (SEQ ID NO:1):

XY¹Y²X′ₘZ¹Z²Z³Z⁴Z⁵

or a pharmaceutically acceptable salt thereof, wherein

X is Ser, Cys, Gly, Arg, R(NMe), Ac-Ser, or Ac-Cys;

Y¹ is any amino acid;

Y² is Cys, Ser, or Asp;

each X′ independently is any amino acid;

Z¹ is Cys, Asp, Thr, or absent;

Z² is Leu, Ile, Val, Lys, or absent;

Z³ is Cys or absent;

Z⁴ is Trp or absent;

Z⁵ is Gln or absent; and

m is 4, 5, 6, or 7 and each X′ may be the same or different.

Another aspect described herein is a compound comprising a peptide scaffold of Formula I, or a pharmaceutically acceptable salt thereof, wherein
X is Ser, Cys, Gly, Arg, R(NMe), Ac-Ser, or Ac-Cys;
Y¹ is Gln, Lys, His, Thr, Glu, Cys, Phe, Met, Asp, Ala, Arg, Ile, Asn, Gly, Leu, Ser, or Trp;
Y² is Cys, Ser, or Asp;
each X′ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;
Z¹ is Cys, Asp, Thr, or absent;
Z² is Leu, Ile, Val, Lys, or absent;
Z³ is Cys or absent;
Z⁴ is Trp or absent;
Z⁵ is Gln or absent; and
m is 4, 5, 6, or 7 and each X′ may be the same or different.

Another aspect described herein is a compound comprising a peptide scaffold having a sequence of Formula (III) (SEQ ID NO: 3):

XY¹Y²X′ₘZ¹Z²

or a pharmaceutically acceptable salt thereof, wherein
X is Ser, Cys, Gly, R(NMe), Ac-Ser, or Ac-Cys;
Y¹ is Gln, Leu, Asp, Lys, Ala, Arg, or Ser;
Y² is Cys, Ser, or Asp;
each X′ independently is His, Pro, Asp, Met, Thr, Nle, Hse(OMe), Ile, Val, Pen, Cys, Trp, Ala, Gly, Tyr, Gln, Glu, Leu, Ser, Cys, Phe, Arg, tBuAla, CbA, Lys, or absent;
Z¹ is Cys or absent;
Z² is Lys or absent; and
m is 4, 5, 6, or 7 and each X′ may be the same or different.

Another aspect described herein is a compound comprising a peptide scaffold having a sequence of Formula (IV) (SEQ ID NO: 4):

XY¹Y²X′ₘZ¹

or a pharmaceutically acceptable salt thereof, wherein
X is Ser, Cys, Gly, R(NMe), or Ac-Cys;
Y¹ is Lys, Arg, or Ser;
Y² is Cys, Ser, or Asp;
each X′ independently is Pro, Asp, Met, Leu, Thr, Nle, Hse(OMe), Ile, Val, Pen, Cys, Trp, Ala, Gly, Tyr, Gln, Glu, Lys, or absent;
Z¹ is Cys or absent; and
m is 4, 5, 6, or 7 and each X′ may be the same or different.

Another aspect described herein are compounds comprising cyclic structures of the peptide scaffolds according to Formula (I) (SEQ ID NO. 1), Formula (III) (SEQ ID NO. 3), or Formula (IV) (SEQ ID NO. 4) may be formed via disulfide bond a) at cysteine at AA3 and at cysteine at AA1-0 (with disulfide bond formation between R₃ and R₁₀), b) at cysteine at AA3 and at cysteine at AA1-1 (with disulfide bond formation between R₃ and R₁₁), or c) at cysteine at AA1 and at cysteine at AA7 (with disulfide bond formation between R₁ and R₇).

Another aspect described herein are compounds comprising cyclic structures of the peptide scaffolds according to Formula (I) (SEQ ID NO. 1), Formula (III) (SEQ ID NO. 3), or Formula (IV) (SEQ ID NO. 4) may also be formed via a sulfide (thioether) bond between a linker ligated at the N-terminal arginine or N-terminal N-methylarginine at AA1 and a cysteine or a penicillamine at AA6.

Another aspect described herein are compounds comprising bicyclic structures of the peptide scaffolds according to Formula (I) (SEQ ID NO. 1), Formula (III) (SEQ ID NO. 3), or Formula (IV) (SEQ ID NO. 4) may be formed via a) covalent bonding of linkers ligated at the N-terminal serine at AA1, at cysteine at AA3 and at cysteine at AA1-0, b) covalent bonding of linkers ligated at the N-terminal serine at AA1, at cysteine at AA3 and at cysteine at AA1-1, or c) covalent bonding of linkers ligated at the N-terminal serine at AA1, at cysteine at AA3 and at cysteine at AA9. Compounds comprising bicyclic structures of the peptide scaffolds according to Formula (I) (SEQ ID NO. 1), Formula (III) (SEQ ID NO. 3), or Formula (IV) (SEQ ID NO. 4) may also be formed via covalent bonding of linkers ligated at the N-terminal glycine at AA1, at cysteine at AA3 and at cysteine at AA11. Furthermore, compounds comprising bicyclic structures of the peptide scaffolds according to Formula (I) (SEQ ID NO. 1), Formula (III) (SEQ ID NO. 3), or Formula (IV) (SEQ ID NO. 4) may also be formed via a sulfide (thioether) bond between a linker ligated at the N-terminal arginine at AA1 and a cysteine at AA7, and a disulfide bond between a cysteine at AA2 and a cysteine at AA13.

Linkers may be of any suitable size, structure and compatibility with any of the compounds described herein, so long as the compounds have the appropriate specificity and conformational stability. Thus, in one aspect of the present disclosure, the linker comprises DIG, polyethylene glycol (PEG) (including PEG of varying molecular weights), IDA, ADA, Boc-IDA, Glutaric acid, Isopthalic acid, 1,3-phenylenediacetic acid, 1,4-phenylenediacetic acid, 1,2-phenylenediacetic acid, Triazine, Boc-Triazine, IDA-biotin, PEG4-biotin, other pegylated carboxylation enhancers, AADA, any suitable aliphatics, aromatics, heteroaromatics, two-fold symmetric linchpin (TSL), PFS, TBMB, DBMB, chloroacetic acid, 3-chloropropanoic acid (Pa), 2-(chloromethyl)benzoic acid (Bz), or other synthetic linkers. In another aspect described herein, the linker comprises a two-fold symmetric linchpin (TSL) linker. In yet another aspect, the linker comprises TSL-1, TSL-3, TSL-6, TSLC-7, DIG, PEG, chloroacetic acid, 3-chloropropanoic acid or 2-(chloromethyl)benzoic acid. Any suitable linker may be used for any of the compounds described herein with the understanding that the necessary chemistry required to facilitate bonding of the linkers at the designated residues is within the knowledge of the skilled artisan.

Exemplary linkers include two-fold symmetric linchpin (TSL) linkers (TSL-1, TSL-3, TSL-6, and TSLC-7) all having thiol-reactive chlorobenzyl functionalities, DIG, PEG, chloroacetic acid, 3-chloropropanoic acid, and 2-(chloromethyl)benzoic acid as described in FIG. 2A.

TSL linkers ligated at specific amino acid residues within any of the peptides described herein may form the bicyclic topology of the compounds of the present disclosure. Prior to ligation, the N-terminal amino acid may be oxidized to an aldehyde to facilitate chemical ligation, thus amino acids may be modified as required. In one aspect described herein, the linkers bond to the peptide scaffold at three amino acid residues within the compound: the free NH₂ on the N-terminal serine (AA1), the cysteine at AA3 (at R₃), and the C-terminal amino acid (for example AA10 or R₁₀). In another aspect described herein, the linker is ligated at the N-terminal serine by an aldehyde reactive oxime functionality. In another aspect described herein, the linker is ligated at the N-terminal glycine by an amide functionality. In another aspect, the linker is ligated to cysteine (AA3 at R₃) and at X′ (AA10 at R₁₀) when X′ is cysteine, each by a thiol-reactive chlorobenzyl functionality. In another aspect, the linker is ligated to cysteine (AA3 at R₃) and at Z¹ (AA11 at R₁₁) when Z¹ is cysteine, each by a thiol-reactive chlorobenzyl functionality. In yet another aspect, the linker is ligated to cysteine (AA3 at R₃) and at X′ (AA9 at R₉) when X′ is cysteine, each by a thiol-reactive chlorobenzyl functionality.

In an aspect described herein, each of 3-chloropropanoic acid (Pa) and 2-(chloromethyl)benzoic acid (Bz) linkers is covalently linked to the peptide scaffold at the N-terminal Arg or R(NMe) by an amide functionality.

In another aspect described herein, a chloroacetic acid linker is covalently linked to the peptide scaffold at an N-terminal Arg by an amide functionality.

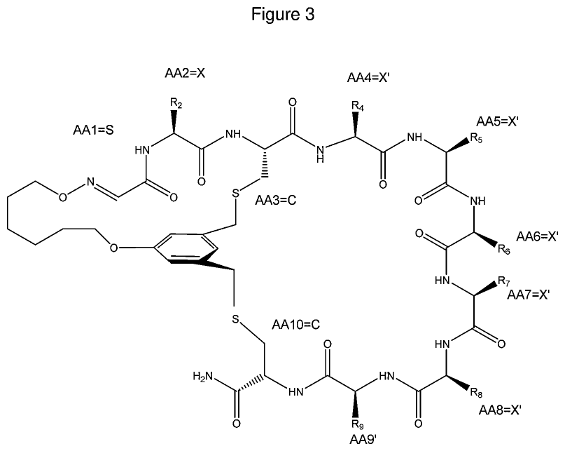

FIG. 3 describes an exemplary compound comprising the bicyclic scaffold of peptides according to Formula (I), Formula (III), or Formula (IV) using the TSL-6 linker.

Contemplating Formula (I), SEQ ID NO:1, Formula (III) SEQ ID NO. 3, and Formula (IV) SEQ ID NO. 4, Table 2 describes the specific sequences for the peptide scaffold of the compounds described herein. Thus, in another embodiment described herein, the compounds comprise a peptide scaffold having any of the sequences described in Table 2, which are specific to α4β7 and/or MAdCAM. While the sequences described herein may be made in any combination of linear, cyclic, or bicyclic with any linkers, the compounds of the present disclosure are cyclic and bicyclic and comprise peptide scaffolds of any peptide sequence and any variety of linkers.

TABLE 2
Peptide Sequences

| SEQ ID NO. | Sequence | Linker | Target |
|---|---|---|---|
| SEQ ID NO. 5 | SMCPLYKVHC | None, cyclic | α4p7 |
| SEQ ID NO. 6 | SACQWYGRMC | TSL-6 | α4P7 |
| SEQ ID NO. 7 | SNCHWYGRIC | TSL-6 | α4P7 |
| SEQ ID NO. 8 | SMCPLYKVHC | TSL-6 | α4P7 |
| SEQ ID NO. 9 | SRCLFNWPDC | TSL-6 | α4P7 |
| SEQ ID NO. 10 | STCFLDVKNC | TSL-6 | α4P7 |
| SEQ ID NO. 11 | SACMLDVISC | TSL-6 | α4P7 |
| SEQ ID NO. 12 | SGCMDDFAFC | TSL-6 | α4P7 |
| SEQ ID NO. 13 | SDCYPDTYHC | None, linear | α4P7 |
| SEQ ID NO. 14 | SDCYPDTYHC | None, cyclic | α4P7 |
| | | (disulfide) | |
| SEQ ID NO. 15 | SECPWPLPQC | TSL-6 | α4P7 |
| SEQ ID NO. 16 | SDCYPDTYHC | TSL-6 | α4P7 |
| SEQ ID NO. 17 | SHCKLDIWVC | TSL-6 | α4P7 |
| SEQ ID NO. 18 | SRCLDIMYDC | TSL-6 | α4P7 |
| SEQ ID NO. 19 | SQCHWYGRSC | TSL-6 | α4P7 |
| SEQ ID NO. 20 | SKCLGWEQTC | TSL-6 | α4P7 |
| SEQ ID NO. 21 | SACQWYGRMC | TSL-6 | α4P7 |
| SEQ ID NO. 22 | SFCLGYEASC | TSL-6 | α4P7 |
| SEQ ID NO. 23 | SQCPMDIPTC | TSL-6 | α4P7 |
| SEQ ID NO. 24 | SRCPMDIAGEC | TSL-6 | α4P7 |
| SEQ ID NO. 25 | SACRSDTLC | TSL-6 | α4P7 |
| SEQ ID NO. 26 | SKCPLDVPSC | TSL-6 | α4P7 |
| SEQ ID NO. 27 | SECYPDTYHC | TSL-6 | α4P7 |
| SEQ ID NO. 28 | SDCFPDTYHC | TSL-6 | α4P7 |
| SEQ ID NO. 29 | SDCYPDTFHC | TSL-6 | α4P7 |
| SEQ ID NO. 30 | SICYPDTYHC | TSL-6 | α4P7 |
| SEQ ID NO. 31 | STCHEMNWKPC | TSL-6 | MAdCAM |
| SEQ ID NO. 32 | STCIYGKFTC | TSL-6 | MAdCAM |
| SEQ ID NO. 33 | STCGPESEKDC | TSL-6 | MAdCAM |
| SEQ ID NO. 34 | SQCGPVKWGHC | TSL-6 | MAdCAM |
| SEQ ID NO. 35 | SWCTKATKNAC | TSL-6 | MAdCAM |
| SEQ ID NO. 36 | SHCRIMEVEQC | TSL-6 | MAdCAM |
| SEQ ID NO. 37 | SMCSYMNTIC | TSL-6 | MAdCAM |
| SEQ ID NO. 38 | SECQENLKYC | TSL-6 | MAdCAM |
| SEQ ID NO. 39 | SACYPDTYHC | TSL-6 | Alanine scan of SEQ ID NO: |
| | | | 15 |
| SEQ ID NO. 40 | SDCAPDTYHC | TSL-6 | Alanine scan of SEQ ID NO: |
| | | | 15 |
| SEQ ID NO. 41 | SDCYADTYHC | TSL-6 | Alanine scan of SEQ ID NO: |
| | | | 15 |
| SEQ ID NO. 42 | SDCYPATYHC | TSL-6 | Alanine scan of SEQ ID NO: |
| | | | 15 |
| SEQ ID NO. 43 | SDCYPDAYHC | TSL-6 | Alanine scan of SEQ ID NO: |
| | | | 15 |
| SEQ ID NO. 44 | SDCYPDTAHC | TSL-6 | Alanine scan of SEQ ID NO: |
| | | | 15 |
| SEQ ID NO. 45 | SDCYPDTYAC | TSL-6 | Alanine scan of SEQ ID NO: |
| | | | 15 |
| SEQ ID NO. 46 | SACPMDIPTC | TSL-6 | Alanine scan of SEQ ID NO: |
| | | | 23 |
| SEQ ID NO. 47 | SQCAMDIPTC | TSL-6 | Alanine scan of SEQ ID NO: |
| | | | 23 |
| SEQ ID NO. 48 | SQCPADIPTC | TSL-6 | Alanine scan of SEQ ID NO: |
| | | | 23 |
| SEQ ID NO. 49 | SQCPMAIPTC | TSL-6 | Alanine scan of SEQ ID NO: |
| | | | 23 |
| SEQ ID NO. 50 | SQCPMDAPTC | TSL-6 | Alanine scan of SEQ ID NO: |
| | | | 23 |
| SEQ ID NO. 51 | SQCPMDIATC | TSL-6 | Alanine scan of SEQ ID NO: |
| | | | 23 |
| SEQ ID NO. 52 | SQCPMDIPAC | TSL-6 | Alanine scan of SEQ ID NO: |
| | | | 23 |
| SEQ ID NO. 53 | SACPLDVPSC | TSL-6 | Alanine scan of SEQ ID NO: |
| | | | 27 |
| SEQ ID NO. 54 | SKCALDVPSC | TSL-6 | Alanine scan of SEQ ID NO: |
| | | | 27 |
| SEQ ID NO. 55 | SKCPADVPSC | TSL-6 | Alanine scan of SEQ ID NO: |
| | | | 27 |
| SEQ ID NO. 56 | SKCPLAVPSC | TSL-6 | Alanine scan of SEQ ID NO: |
| | | | 27 |
| SEQ ID NO. 57 | SKCPLDAPSC | TSL-6 | Alanine scan of SEQ ID NO: |
| | | | 27 |
| SEQ ID NO. 58 | SKCPLDVASC | TSL-6 | Alanine scan of SEQ ID NO: |
| | | | 27 |
| SEQ ID NO. 59 | SKCPLDVPAC | TSL-6 | Alanine scan of SEQ ID NO: |
| | | | 27 |
| SEQ ID NO. 60 | SDCHDKFTEC | TSL-6 | α4p7 |
| SEQ ID NO. 61 | SDCHDPPKVC | TSL-6 | α4P7 |
| SEQ ID NO. 62 | SDCPDSMAHSC | TSL-6 | α4P7 |
| SEQ ID NO. 63 | SDCPDAERYIC | TSL-6 | α4P7 |
| SEQ ID NO: 64 | RSDTLCWK | thioether | α4P7 |
| SEQ ID NO: 65 | RSDTL(Pen)WE | thioether | α4P7 |
| SEQ ID NO: 66 | CRSDTLCGE | None (disulfide) | α4P7 |
| SEQ ID NO: 67 | CRSDTLC | None (disulfide) | α4P7 |
| SEQ ID NO: 68 | SACRSDTLCG | TSL-6 | α4P7 |
| SEQ ID NO: 69 | CRSDTLC | cyclic, disulfide | α4P7 |
| SEQ ID NO: 70 | SACRSDTLC | TSL-1 | α4P7 |
| SEQ ID NO: 71 | SKCPADTPYEC | TSL-6 | α4P7 |
| SEQ ID NO: 72 | SKCPLATPYEC | TSL-6 | α4P7 |
| SEQ ID NO: 73 | SHCKLDIWVC | TSL-6 | α4P7 |
| SEQ ID NO: 74 | STCRMDFQDC | TSL-6 | α4P7 |
| SEQ ID NO: 75 | SECPWPLPQC | TSL-6 | α4P7 |
| SEQ ID NO: 76 | RCS{Nle}DICRS | Cyclic | α4P7 |
| | {NIe}DICWQ | (disulfide) | |
| SEQ ID NO: 77 | SFCQMFEKAC | TSL-6 | α4P7 |
| SEQ ID NO: 78 | SMCYPDTYHC | TSL-6 | α4P7 |
| SEQ ID NO: 79 | SFCGATMKKC | TSL-6 | α4P7 |
| SEQ ID NO: 80 | SQCP{Ox- | TSL-6 | α4P7 |
| | Met}AIPTC | | |
| SEQ ID NO: 81 | SFCLGYEASC | TSL-6 | α4P7 |
| SEQ ID NO: 82 | SDCYPDFYHC | TSL-6 | α4P7 |
| SEQ ID NO: 83 | SDCWPDTYHC | TSL-6 | α4P7 |
| SEQ ID NO: 84 | SRCLDIMYDC | TSL-6 | α4P7 |
| SEQ ID NO: 85 | SACRSDTLC | TSL-6 | α4P7 |
| SEQ ID NO: 86 | SNCYPDTYHC | TSL-6 | α4p7 |
| SEQ ID NO: 87 | RCSDTLCRSLDVC | Cyclic | α4p7 |
| | WQ | (disulfide) | |
| SEQ ID NO: 88 | SACSDTGLQC | TSL-6 | α4p7 |
| SEQ ID NO: 89 | GQCP{Hse(OMe)} | TSLC-7 | α4p7 |
| | D{tBuAla}PQEC | | |
| SEQ ID NO: 90 | RCS{Nle}DICRSDT | Bicyclic | α4p7 |
| | LCWQ | (thioether and | |
| | | disulfide) | |
| SEQ ID NO: 91 | SACPMDVWGEC | TSL-6 | α4p7 |
| SEQ ID NO: 92 | SLCYPDTYHC | TSL-6 | α4p7 |
| SEQ ID NO: 93 | SMCPLYIVHC | TSL-6 | α4p7 |
| SEQ ID NO: 94 | RCS{Nle}DICRSLD | Bicyclic | α4p7 |
| | VCWQ | (thioether and | |
| | | disulfide) | |
| SEQ ID NO: 95 | SDCPWQSKTC | TSL-6 | α4p7 |
| SEQ ID NO: 96 | RCSDTLCRS{Nle} | Bicyclic | α4p7 |
| | DICWQ | (thioether and | |
| | | disulfide) | |
| SEQ ID NO: 97 | SQCP{Ox- | TSL-6 | α4p7 |
| | Met}DAPTC | | |
| SEQ ID NO: 98 | SICRTDVPIDC | TSL-6 | α4p7 |
| SEQ ID NO: 99 | SQCP{Nle}DIATC | TSL-6 | α4p7 |
| SEQ ID NO: 100 | SLCYEWEHAC | TSL-6 | α4p7 |
| SEQ ID NO: 101 | SACPMDVAGEC | TSL-6 | α4p7 |
| SEQ ID NO: 102 | SDCHDKFTEC | TSL-6 | α4p7 |
| SEQ ID NO: 103 | SRCP{tBuAla}DVW | TSL-6 | α4p7 |
| | GEC | | |
| SEQ ID NO: 104 | SACAWHQHAC | TSL-6 | α4p7 |
| SEQ ID NO: 105 | SRCP{CbA}D{tBuA | TSL-6 | α4p7 |
| | la}WQEC | | |
| SEQ ID NO: 106 | SRCP{Nle}D{CbA} | TSL-6 | α4p7 |
| | WQEC | | |
| SEQ ID NO: 107 | SACPMDVWQEC | TSL-6 | α4p7 |
| SEQ ID NO: 108 | SKCALDTPYEC | TSL-6 | α4p7 |
| SEQ ID NO: 109 | Ac- | Cyclic dimer | α4p7 |
| | SRCPMDIAGECK | (BisPEG5 and | |
| | | disulfide) | |
| SEQ ID NO: 110 | SRCP{Hse(OMe)}D | TSL-6 | α4p7 |
| | IWYEC | | |
| SEQ ID NO: 111 | Ac-SQCPMDIPTC | Cyclic | α4p7 |
| | | (disulfide) | |
| SEQ ID NO: 112 | GKCP{Nle}D{CbA} | TSLC-7 | α4p7 |
| | WQEC | | |
| SEQ ID NO: 113 | SACPLDTPYEC | TSL-6 | α4p7 |
| SEQ ID NO: 114 | SKCPLDAPYEC | TSL-6 | α4p7 |
| SEQ ID NO: 115 | SKCPLDVPSC | Cyclic | α4p7 |
| | | (disulfide) | |
| SEQ ID NO: 116 | SQCPMDIPTC | TSL-6 | α4p7 |
| SEQ ID NO: 117 | SRCP{Nle}D{tBuAI | TSL-6 | α4p7 |
| | a}PYEC | | |
| SEQ ID NO: 118 | SKCPLDTAYEC | TSL-6 | α4p7 |
| SEQ ID NO: 119 | Ac- | Cyclic | α4p7 |
| | SRCPMDIAGEC | (disulfide) | |
| SEQ ID NO: 120 | SKCPLDTPAEC | TSL-6 | α4p7 |
| SEQ ID NO: 121 | SKCPLDTPTEC | TSL-6 | α4p7 |
| SEQ ID NO: 122 | GACP{Nle}DVWQE | TSLC-7 | α4p7 |
| | C | | |
| SEQ ID NO: 123 | Ac- | Cyclic dimer | α4p7 |
| | SRCPMDIAGECK | (DIG and | |
| | | disulfide) | |
| SEQ ID NO: 124 | SRCP{tBuAla}DVP | TSL-6 | α4p7 |
| | QEC | | |
| SEQ ID NO: 125 | SKCPLDMPYEC | TSL-6 | α4p7 |
| SEQ ID NO: 126 | SQCPMDVPTC | TSL-6 | α4p7 |
| SEQ ID NO: 127 | SRCP{Nle}DIWVK | TSL-6 | α4p7 |
| | C | | |
| SEQ ID NO: 128 | SKCPLDTPQEC | TSL-6 | α4p7 |
| SEQ ID NO: 129 | SKCPLDTPYAC | TSL-6 | α4p7 |
| SEQ ID NO: 130 | SRCPMDVAGEC | TSL-6 | α4p7 |
| SEQ ID NO: 131 | SKCPLDTPYEC | TSL-6 | α4p7 |
| SEQ ID NO: 132 | GKCP{Nle}DIWVK | TSLC-7 | α4p7 |
| | C | | |
| SEQ ID NO: 133 | SRCPMDIPTC | TSL-6 | α4p7 |
| SEQ ID NO: 134 | SKCPLDLPYEC | TSL-6 | α4p7 |
| SEQ ID NO: 135 | SKCPMDIPTC | TSL-6 | α4p7 |
| SEQ ID NO: 136 | SRCPLDTPYEC | TSL-6 | α4P7 |
| SEQ ID NO: 137 | SRCPMDIWQEC | TSL-6 | α4P7 |
| SEQ ID NO: 138 | SRCP{Nle}DVAGE | TSL-6 | α4P7 |
| | C | | |
| SEQ ID NO: 139 | SRCP{Hse(Ome)}D | TSL-6 | α4P7 |
| | IWQEC | | |
| SEQ ID NO: 140 | SRCPMDIAGEC | TSL-6 | α4P7 |
| SEQ ID NO: 141 | SRCP{Nle}DIAAEC | TSL-6 | α4p7 |
| SEQ ID NO: 142 | SKCPLDIPYEC | TSL-6 | α4p7 |
| SEQ ID NO: 143 | SKCPLDVPYEC | TSL-6 | α4p7 |
| SEQ ID NO: 144 | SRCP{Nle}DIWQE | TSL-6 | α4p7 |
| | C | | |
| SEQ ID NO: 145 | SRCP{Nle}DVWYE | TSL-6 | α4p7 |
| | C | | |
| SEQ ID NO: 146 | Ac-CRSDTLC | Cyclic | α4p7 |
| | | (disulfide) | |
| SEQ ID NO: 147 | GRCP{Nle}DIWQE | TSLC-7 | α4p7 |
| | C | | |
| SEQ ID NO: 148 | SRCP{Nle}DVWQE | TSL-6 | α4p7 |
| | C | | |
| SEQ ID NO: 149 | Pro{R(NMe)}SDTL | Cyclic | α4p7 |
| | CWK | (thioether) | |
| SEQ ID NO: 150 | Bz{R(NMe)}SDTL | Cyclic | α4p7 |
| | (Pen)WE | (thioether) | |
| SEQ ID NO: 151 | Ac-CRSDTLCGE | cyclic | α4p7 |
| | | (disulfide) | |
| SEQ ID NO: 152 | SKCPLDTPYEC | cyclic | α4p7 |
| | | (disulfide) | |

The compounds described herein include a peptide scaffold, wherein the sequences of the peptide scaffold have specificity for α4β7 and/or MAdCAM. It is well known that the ‘C-D’ loop structure in MAdCAM in which α4β7 binds includes an “LDT” amino acid motif thought to be primarily responsible for binding specificity to α4β7. Thus, without being bound by any theory, it is believed that within peptide scaffold described herein, amino acid residues from AA4-AA7 having the sequences with the highest homology to the loop in MAdCAM may have significance in determining the specificity of the compounds described herein for α4β7. Thus, scaffold sequences having the motif “LDV” (SEQ ID NOs. 10, 11 and 26), “LDI” (SEQ ID NOs. 17 and 18) and “LDT” (SEQ ID NO: 131) are being studied to determine if these motifs result in enhanced specificity for the α4β7 target.

Exemplary peptide scaffolds include scaffolds A, B, C, D, E, F, G, H, I, J A-S, B-S, C-S, D-S, E-S, F-S, G-S, H-S, I-S, and J-S as follows.

Scaffold A and A-S each contains the TSL-6 linker covalently linked to an N-terminal serine at AA1, a Cys at AA3, and a Cys at AA11.

Scaffold B and B-S each contains the TSLC-7 linker covalently linked to an N-terminal glycine at AA1, a Cys at AA3, and a Cys at AA11.

Scaffold C and C-S each contains the TSL-1 linker covalently linked to an N-terminal serine at AA1, a Cys at AA3, and a Cys at AA9.

Scaffold D and D-S each contains a disulfide bond that links a Cys at AA1 and a Cys at AA7. As an example, D is delineated to show the location of the individual amino acids.

Scaffold E and E-S each contains a disulfide bond that links a Cys at AA3 and a Cys at AA11.

Scaffold F and F-S are bicyclic and each scaffold contains a sulfide (thioether) bond between a R at AA1 and a Cys at AA7 and a disulfide bond between a Cys at AA2 and a Cys at AA13.

Scaffold G and G-S each contains a thioether bond between R(NMe) at AA1 and a Cys at AA6.

Scaffold H and H-S each contains a thioether bond between R(NMe) at AA1 and a Pen at AA6.

Compounds comprising two peptide scaffolds according to Formula (I) (SEQ ID NO. 1), Formula (III) (SEQ ID NO. 3), or Formula (IV) (SEQ ID NO. 4) may be formed via a PEG (scaffold I and I-S) or DIG (scaffold J and J-S) linker bound to the first peptide scaffold at a Lys at AA12 by an amide functionality, and bound to the second peptide scaffold at a Lys at AA12 by an amide functionality. The exemplary peptide scaffolds are described below:

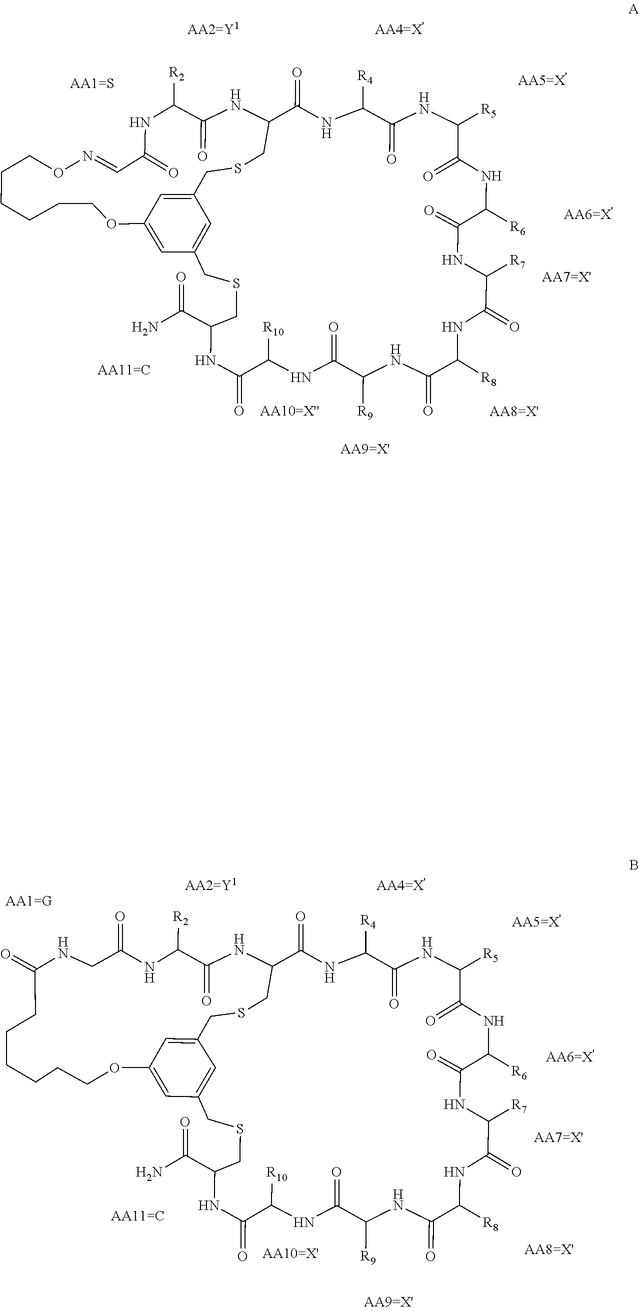

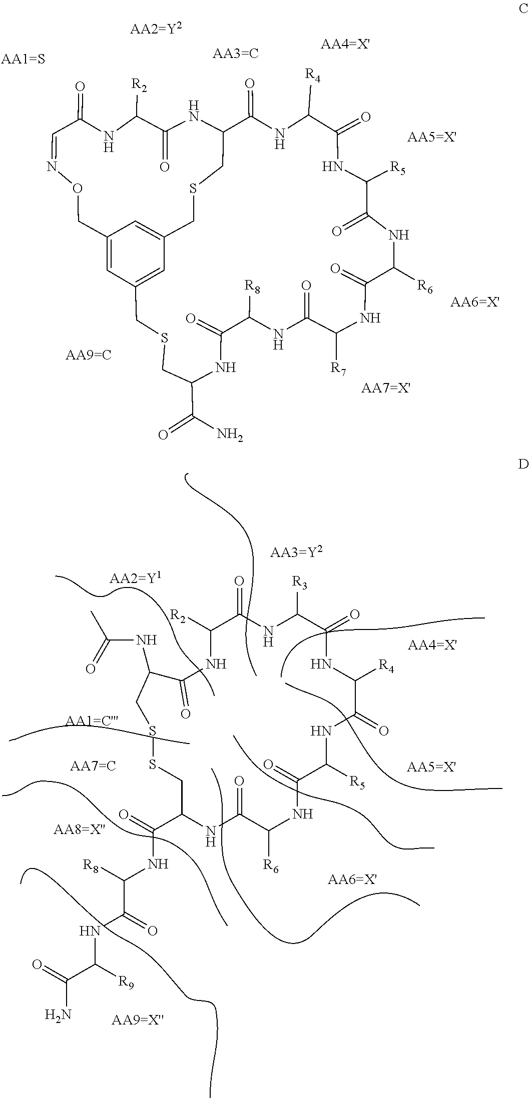

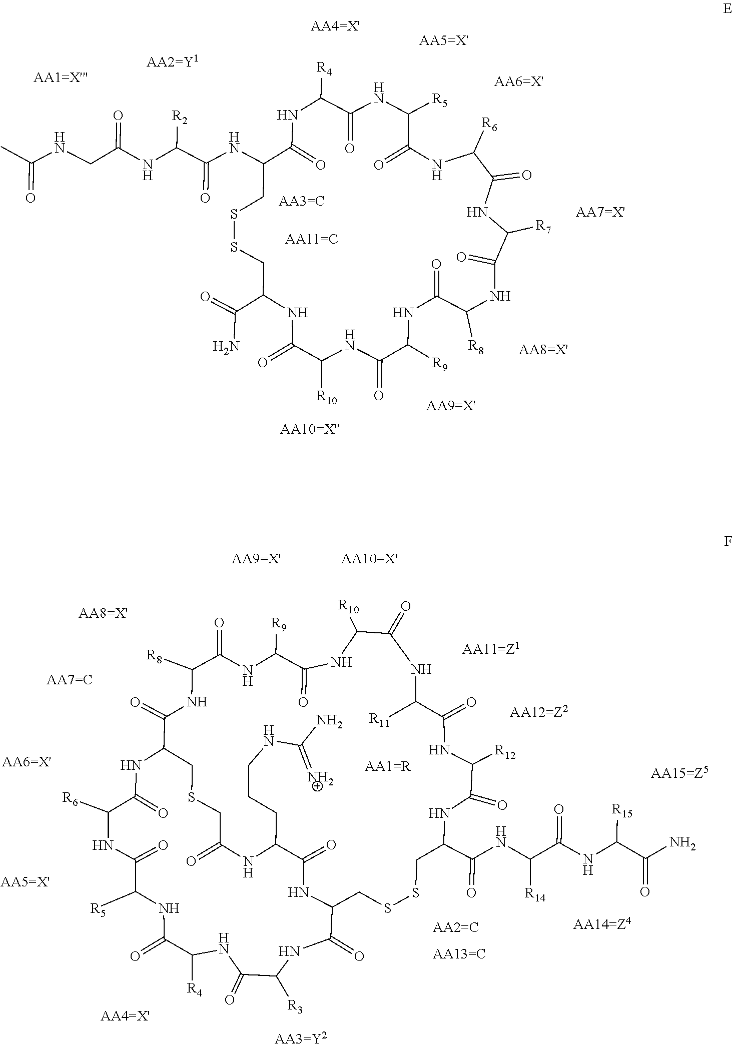

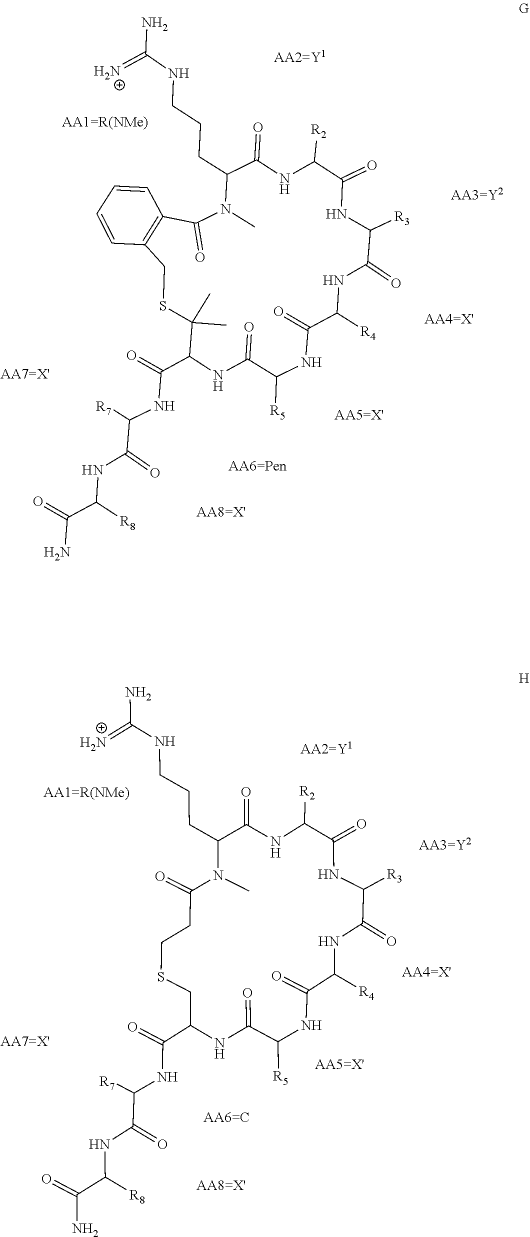

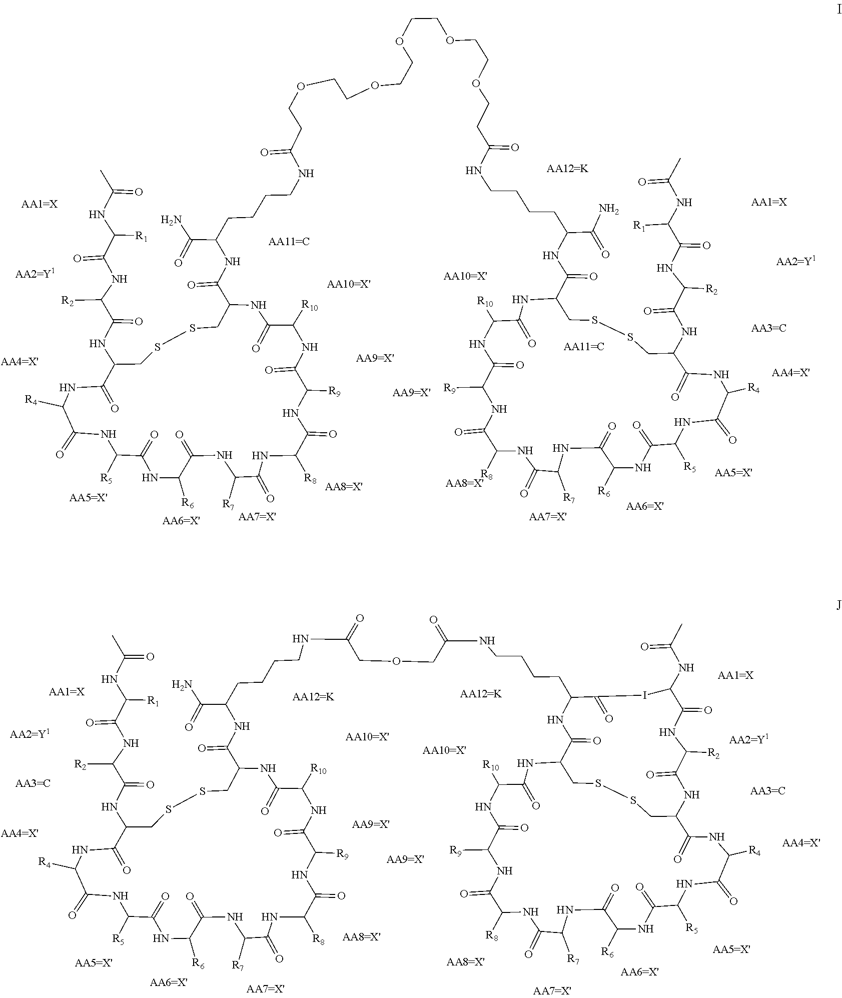

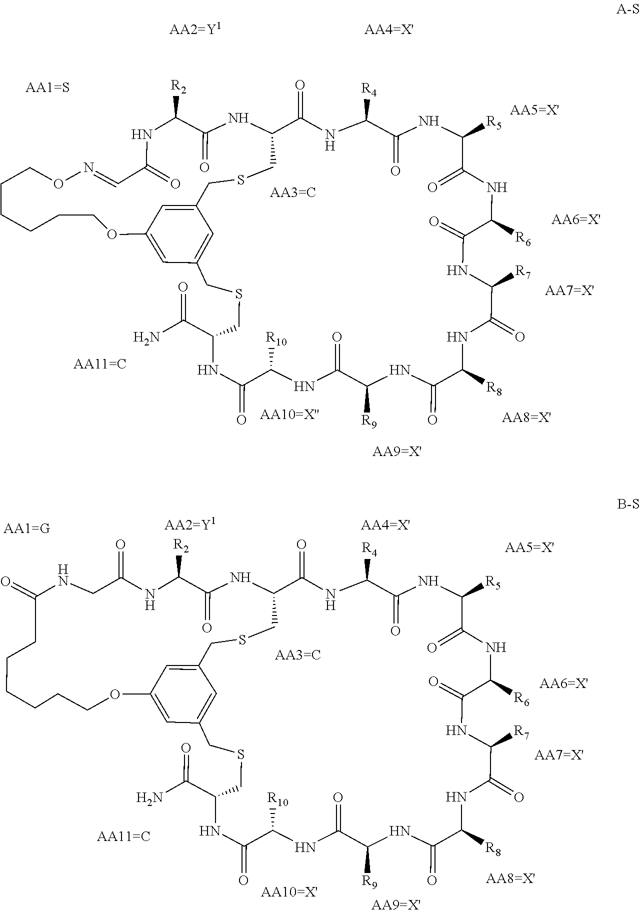

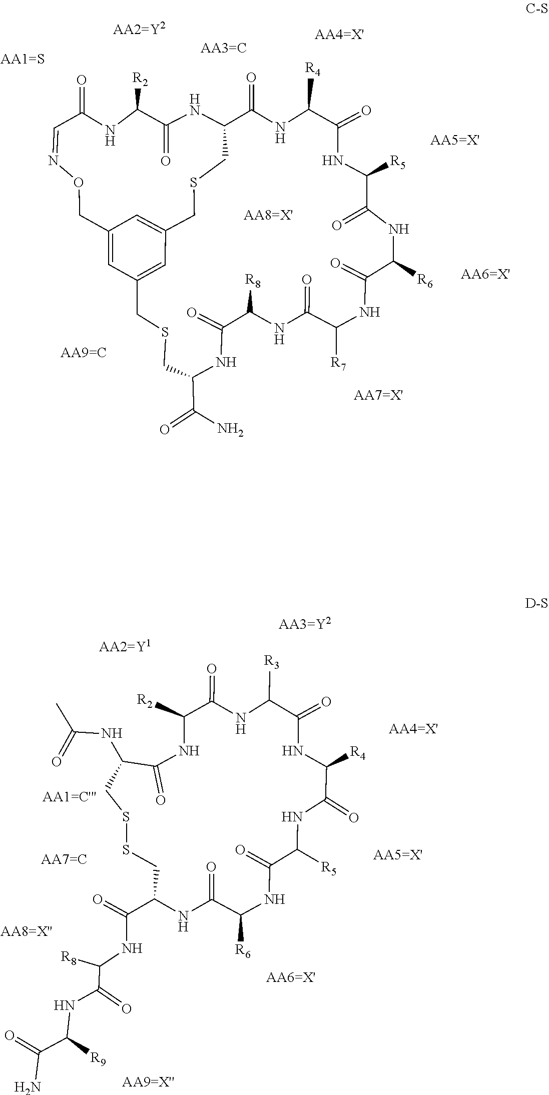

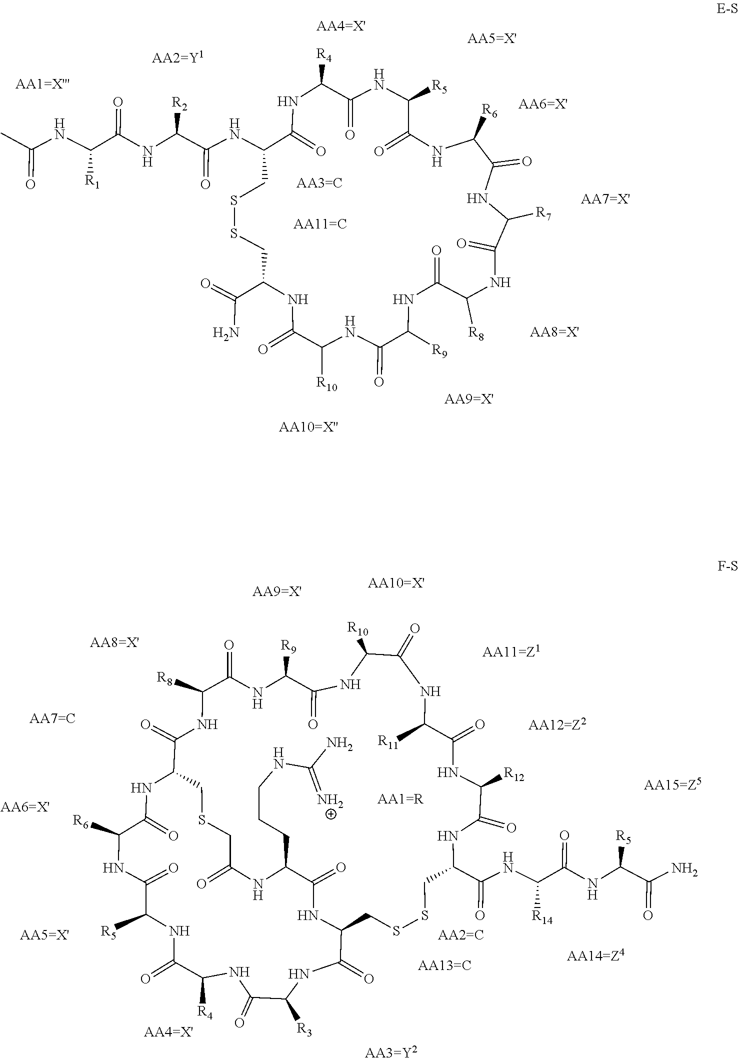

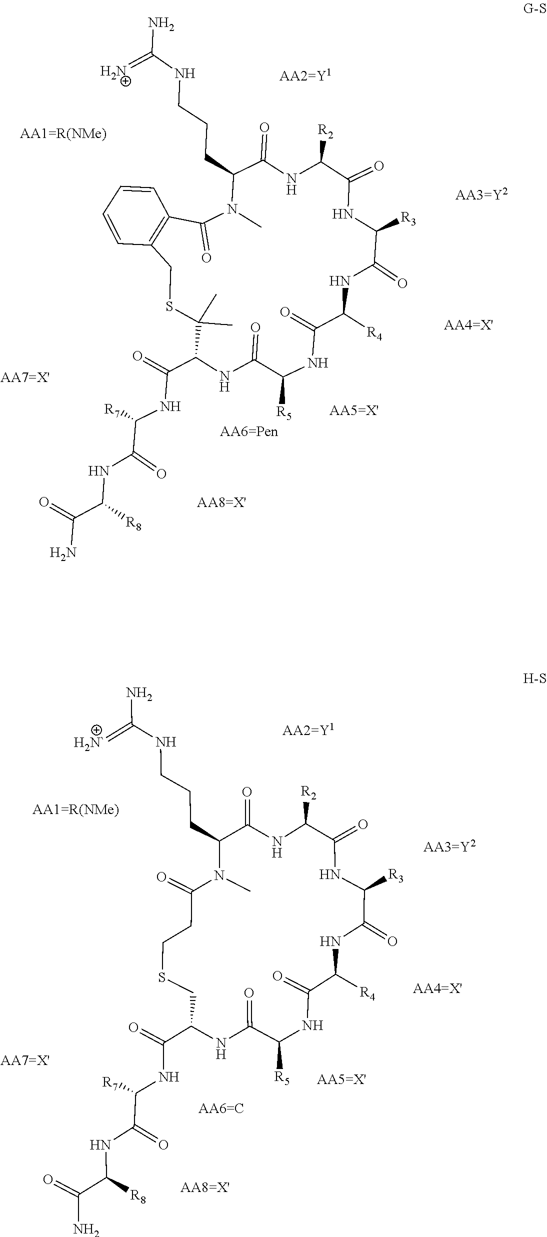

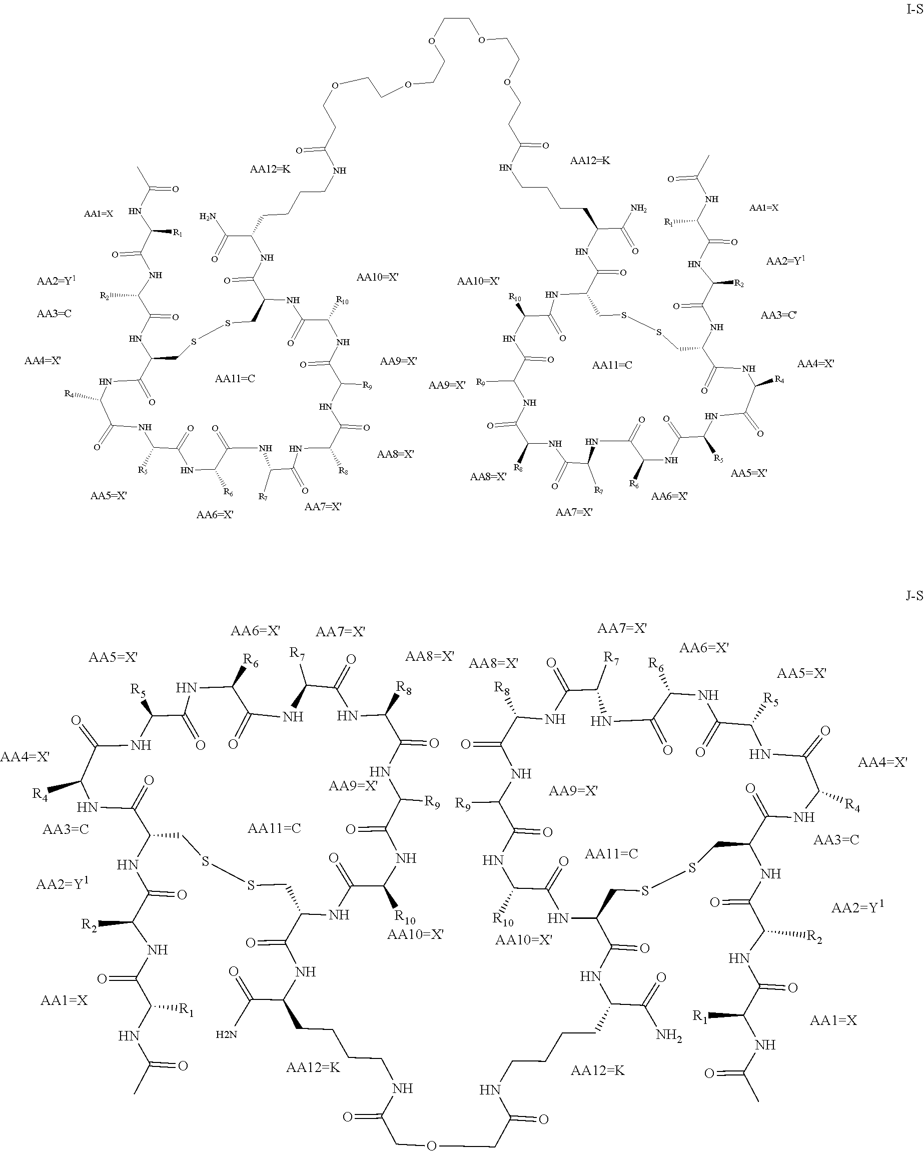

wherein
each X independently is Ser, Cys, Gly, Arg, R(NMe), Ac-Ser, or Ac-Cys;
each Y¹ independently is any amino acid;
each Y² independently is any amino acid;
each X′ independently is any amino acid;
each X″ independently is any amino acid or absent;
each C′″ independently is Cys or an Ac-Cys;
each X′″ independently is any amino acid;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In another aspect, the peptide scaffolds are scaffold A, B, C, D, E, F, G, H, I, J A-S, B-S, C-S, D-S, E-S, F-S, G-S, H-S, I-S, and J-S, wherein
each X independently is Ser, Cys, Gly, Arg, R(NMe), Ac-Ser, or Ac-Cys;
each Y¹ independently is Gln, Lys, His, Thr, Glu, Cys, Phe, Met, Asp, Ala, Arg, Ile, Asn, Gly, Leu, Ser, or Trp;
each Y² independently is Cys, Ser, or Asp;
each X′ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;
each X″ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, Pen, or absent;
each C′″ independently is Cys or an Ac-Cys;
each X′″ independently is Ser, Cys, Gly, Arg, R(NMe), Ac-Ser, or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

In another aspect, the peptide scaffolds are scaffold A, B, C, D, E, F, G, H, I, J A-S, B-S, C-S, D-S, E-S, F-S, G-S, H-S, I-S, and J-S, wherein each X independently is Ser, Cys, Gly, Arg, R(NMe), Ac-Ser, or Ac-Cys;
each Y¹ independently is Lys, Arg, or Ser;
each Y² independently is Cys, Ser, or Asp;
each X′ independently is Pro, Asp, Met, Leu, Thr, Nle, Hse(OMe), Ile, Val, Pen, Cys, Trp, Ala, Gly, Tyr, Gln, Glu, Lys, or absent;
each X″ independently is Pro, Asp, Met, Leu, Thr, Nle, Hse(OMe), Ile, Val, Pen, Cys, Trp, Ala, Gly, Tyr, Gln, Glu, Lys, or absent;
each C′″ independently is Cys or an Ac-Cys;
each X′″ independently is Ser, Cys, Gly, R(NMe), or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

The compounds of the present disclosure may also be utilized to achieve the desired pharmacological effect by administration to a patient in need thereof in an appropriately formulated pharmaceutical composition. A patient, for the purpose of this disclosure, is a mammal, including a human, in need of treatment for a particular condition or disease. Therefore, the present disclosure includes pharmaceutical compositions which include a pharmaceutically acceptable carrier and a therapeutically effective amount of any of the compounds as described herein. One embodiment described in the present disclosure provides compositions that modulate α4β7 and/or MAdCAM activity. Generally, the compositions that modulate activity in humans and animals will comprise a pharmaceutically acceptable carrier or diluent and any of the compounds described herein.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, nasally or the like. In some aspects, the compound is administered intravenously, orally, buccally, or transdermally.

A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated.

In one embodiment described herein is a composition comprising one or more compounds as described herein and one or more pharmaceutically acceptable carriers.

The term “composition” as used herein is intended to encompass a product comprising specific ingredients in specified amounts, as well as any product which results, directly or indirectly, from combination of the specified ingredients in the specified amounts. By “pharmaceutically acceptable”, it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy. All methods include the step of bringing the active ingredient into association with a carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which may be of the ordinary hard- or soft-shelled gelatin type containing, for example, surfactants, lubricants, and inert fillers such as lactose, sucrose, calcium phosphate, and corn starch.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

In another aspect, the compounds of this disclosure may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Such suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n propyl, phydroxybenzoate, one or more coloring agents, one or more flavoring agents, and one or more sweetening agents, such as sucrose or saccharin.

The pharmaceutical compositions of this disclosure may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Sweetening agents such as those set forth above, and flavoring agents may be added to provide a palatable oral preparation. These compositions may be preserved by the addition of an antioxidant such as ascorbic acid.

Syrups and elixirs may be formulated with sweetening agents, for example glycerol, propylene glycol, sorbitol or sucrose. Such formulations may also contain a demulcent, a preservative, and flavoring and coloring agents. Oral solutions can be prepared in combination with, for example, cyclodextrin, PEG and surfactants.

The compounds of the instant disclosure may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

The parenteral compositions of this disclosure may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant may be a single component having the above HLB or may be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3 butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono or diglycerides. In addition, fatty acids such as oleic acid find use in the preparation of injectables.

Another formulation employed in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art (See, e.g., U.S. Pat. No. 5,023,252, incorporated herein by reference). Such patches may be constructed for continuous, pulsatile, or on demand delivery of pharmaceutical agents.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Additionally, the compounds can be administered via ocular delivery by means of solutions or ointments. Still further, transdermal delivery of the subject compounds can be accomplished by means of iontophoretic patches and the like.

For topical use, creams, ointments, jellies, solutions or suspensions containing the compounds of the present disclosure are employed. As used herein, topical application is also meant to include the use of mouth washes and gargles.

Methods of delivering the compounds of this disclosure include any number of modes of administering the compounds or pharmaceutical compositions of compounds to the lungs via the mouth or rectum. Modes of administration may include delivery of liquid or powder formulations of pharmaceutical compositions for nasal administration via either passive of active delivery mechanisms. Liquid formulations may be delivered through a variety of mechanisms including vaporization through nasal inhalation, hand actuated nasal devices and mechanical spray pumps. Formulations for such delivery mechanisms may be in the form of propellant containing aerosols or propellant-free inhalable solutions. Mechanical spray pumps may be hand actuated, gas driven or electrical, as in the case of electrically powered nebulizers and atomizers. In one aspect described herein, a propellant-free inhalable solution is administered by nebulizer or direct nasal inhalation.

Powder formulations may be delivered through mechanical power sprayers, nasal inhalers and nebulizers/atomizers. Prior to delivery, powder formulations may be solubilized in suitable solvents including water and saline solutions. In one aspect described herein the compound may be solubilized in a saline solution. In another aspect described herein a therapeutically effective amount of the compound delivered to the lungs.

The compositions of the disclosure may also contain other conventional pharmaceutically acceptable compounding ingredients, generally referred to as carriers or diluents, as necessary or desired. Any of the compositions of this disclosure may be preserved by the addition of an antioxidant such as ascorbic acid or by other suitable preservatives. Conventional procedures for preparing such compositions in appropriate dosage forms may be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

In yet another aspect of the present disclosure is method of treating a disease or condition associated with the biological function of α4β7 comprising administering to a subject in need thereof an effective amount of any of the compound of the present disclosure. Compounds for use in the present methods include those compounds according to Formula (I), Formula (III), Formula (IV), SEQ ID Nos. 1-152, and those provided above as embodiments, those specifically exemplified in the Examples below, and those provided with specific sequences herein. In another embodiment described herein, is method of treating a disease or condition associated with the biological function of MAdCAM comprising administering to a subject in need thereof an effective amount of any of the compounds of the present disclosure, including the compounds according to Formula (I), Formula (III), Formula (IV), or Table 2. Compounds for use in the present methods include those compounds according to Formula (I), Formula (III), Formula (IV), and Table 2, and those provided above as embodiments, those specifically exemplified in the Examples below, and those provided with specific sequences herein.

In another aspect of the present disclosure is a compound for use for treating a disease or condition associated with the biological function of α4β7 comprising administering to a subject in need thereof an effective amount of any of the compound of the present disclosure.

In another embodiment described herein, is a compound for use for treating a disease or condition associated with the biological function of MAdCAM comprising administering to a subject in need thereof an effective amount of any of the compounds of the present disclosure, including the compounds according to Formula (I), Formula (III), Formula (IV), or Table 2. Compounds for use in the present methods include those compounds according to Formula (I), Formula (III), Formula (IV), and Table 2, and those provided above as embodiments, those specifically exemplified in the Examples below, and those provided with specific sequences herein. In another aspect of the present disclosure is a use of a compound in the treatment of a disease or condition associated with the biological function of α4β7 comprising administering to a subject in need thereof an effective amount of any of the compound of the present disclosure.

In another embodiment described herein, is a use of a compound in the treatment of a disease or condition associated with the biological function of MAdCAM comprising administering to a subject in need thereof an effective amount of any of the compounds of the present disclosure, including the compounds according to Formula (I), Formula (III), Formula (IV), or Table 2. Compounds for use in the present methods include those compounds according to Formula (I), Formula (III), Formula (IV), and Table 2, and those provided above as embodiments, those specifically exemplified in the Examples below, and those provided with specific sequences herein.

As used herein the term “biological function” contemplates any activity or interaction of any of the molecules described herein, examples of which include, but not limited to, binding to any receptors, ligands or binding partners on the same or different cells, and other interactions as understood by those skilled in the art. Modulation of biological function thus contemplates any type of regulation, balancing, adjusting of any biological function, including but not limited to, antagonists, agonists, allosteric modulators, upregulating, downregulating, promoting, suppressing, enhancing, inhibiting, activating, and others as understood by those skilled in the art. Thus, without being bound by any theory, an antagonist of α4β7 would block α4β7 from binding to any binding partner or ligand, including MAdCAM. Similarly, an antagonist of MAdCAM would block MAdCAM from binding to its binding partner or ligand, including α4β7 integrin.

Yet another aspect described herein, is a method of treating disease comprising administrating to a subject in need thereof an effective amount of any of the compounds described herein. In one aspect, diseases include but are not limited to Inflammatory Bowel Disease (IBD), ulcerative colitis, Crohn's disease, Celiac disease, colitis, diverticulitis, eosinophilic gastroenteritis, gastrointestinal cancer, autoimmune hepatitis, pancreatitis, encephalomyelitis, psoriasis and other inflammatory dermatoses such as dermatitis, eczema, atopic dermatitis, allergic contact dermatitis, urticarial, and pruritus, autoimmune diseases, such as fibromyalgia, scleroderma, ankylosing spondylitis, juvenile RA, Still's disease, polyarticular juvenile RA, pauciarticular juvenile RA, polymyalgia rheumatica, rheumatoid arthritis (RA), psoriatic arthritis, osteoarthritis, polyarticular arthritis, multiple sclerosis, lupus, systemic lupus erythematosus, type I diabetes (T1DM), type II diabetes (T2DM), glomerulonephritis and the like, graft-v-host disease (including both acute and chronic), HIV infection, other diseases in which undesired inflammatory responses are to be inhibited.

In another aspect described herein, is a compound for use for treating disease comprising administrating to a subject in need thereof an effective amount of any of the compounds described herein.

In another aspect described herein, is a use of a compound in the treatment of a disease comprising administrating to a subject in need thereof an effective amount of any of the compounds described herein.

In one aspect, the disease comprises Inflammatory Bowel Disease, ulcerative colitis, and Crohn's disease.

In another aspect, the compounds of the present disclosure may be administered in combination with one or more additional therapeutic agent to improve the efficacy of other drugs. Potential other drugs include but not limited to: chemotherapeutic drugs including but not limited to camptothecin, indolizino, irinotecan, diflomotecan, exatecan, gimatecan, irinotecan, karenitecin, lurtorecan, rubitecan, silatecan, topotecan; NSAIDs including but not limited to salicylates, p-amino phenol derivatives, propionic acid derivatives, carboxylic acid derivatives, enolic acid derivatives, fenamic acid derivatives, sulphonanilides, and selective COX-2 inhibitors. “NSAID salicylates” include, but are not limited to, aspirin (acetylsalicylic acid), diflunisal, and salsalate. “NSAID p-amino phenol derivatives” include, but are not limited to, paracetamol and phenacetin. “NSAID propionic acid derivatives” include, but are not limited to, ibuprofen, naproxen, fenoprofen, ketoprofen, dexketoprofen, fluribiprofen, oxaprozin, and loxoprofen. “NSAID carboxylic acid derivatives” include, but are not limited to, indomethacin, sulindac, etodolac, ketorolac, diclofenac. NSAID carboxylic acid derivatives include NSAID acetic acid derivatives. NSAID carboxylic acid derivatives are also referred to herein as “carboxylic acid NSAIDs.” “NSAID enolic acid derivatives” include, but are not limited to, piroxicam, meloxicam, tenoxicam, droxicam, lomoxicam, and isoxicam. “NSAID fenamic acid derivatives” include, but are not limited to, mefenamic acid, meclofenamic acid, flufenamic acid, and tolfenamic acid. “NSAID sulphonanilides” include, but are not limited to, nimesulide. “NSAID selective COX-2 inhibitors” include, but are not limited to, celecoxib, rofecoxib, valdecoxib, parecoxib, lumiracoxib, etoricoxib, and firocoxib; antibiotics including not limited to cephalosporins such as cefixime and cefpodoxime, clindamycin, penicillins, fluoroquinolones such as ciprofloxacin and levofloxacin; immunosuppressants and anti-rejection drugs including but not limited to tacrolimus and cyclosporine, mycophenolate mofetil, mycophenolate sodium, azathioprine, sirolimus and prednisone; other β-glucuronidase substrate drugs including but not limited to morphine, paracetamol, oxazepam, androsterone, carbamazepine, codeine, lamotrigine, lorazepam, temazepam, testosterone, and zidovudine; analgesics including but not limited to analgesics suitable for use in the pharmaceutical compositions described herein include, for example, opioids, natural opium alkaloids, morphine, opium, hydromorphone, nicomorphine, oxycodone, dihydrocodeine, diamorphine, tapentadol, papavereturn, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics such as, for example, rimazolium, glafenine, floctafenine, viminol, nefopam, flupirtine, or ziconotide.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

The therapeutically effective dosage of the compounds of this disclosure may readily be determined for treatment of each desired indication. The amount of the active ingredient (e.g., compounds) to be administered in the treatment of one of these conditions may vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg. The daily inhaled concentration may be that required to maintain a daily dose of from 0.0001 mg/kg to 10 mg/kg.

The specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present disclosure may be ascertained by those skilled in the art using conventional treatment tests.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### Examples

The following examples describe the synthesis, purification, cyclization of the compounds described herein, including various assays to determine biophysical characteristics. It is understood that the skilled artisan may modify any of the examples, protocols and procedures in order to generate and/or test suitable compounds as described herein.

### Synthesis and Purification of Peptides

The compounds of the present disclosure may be made and purified by any means known in the art, including by the use of commercially available protein synthesizers for making the peptide scaffolds described herein. Alternatively, the peptides may be commercially purchased. The peptides of Table 2 were purchased from Genscript® and/or DeliverTides.

Alternatively, exemplary peptide synthesis may be performed using solid phase peptide synthesis with Fmoc chemistry on a commercial peptide synthesizer. Peptides may be cleaved using trifluoroacetic acid (TFA) and purified using a C18 column on a Gilson® Semiprep HPLC. The peptides may be lyophilized and the mass confirmed using MALDI-TOF mass spectrometry.

It is understood that the skilled artisan may use any means of synthesis and purification necessary to obtain any suitable peptide scaffolds including the peptide scaffolds described herein.

### Synthesis of Linkers

Chemical linkers as described in the present disclosure may be synthesized and purified by any means known in the art, including, for example, as described in Wong et al., 2021, or as described herein. Alternatively, linkers may be purchased.

The chemical linkers TSL-1, TSL-3 and TSL-6 containing aminooxy and benzyl chloride functional groups were synthesized according to Scheme 1:

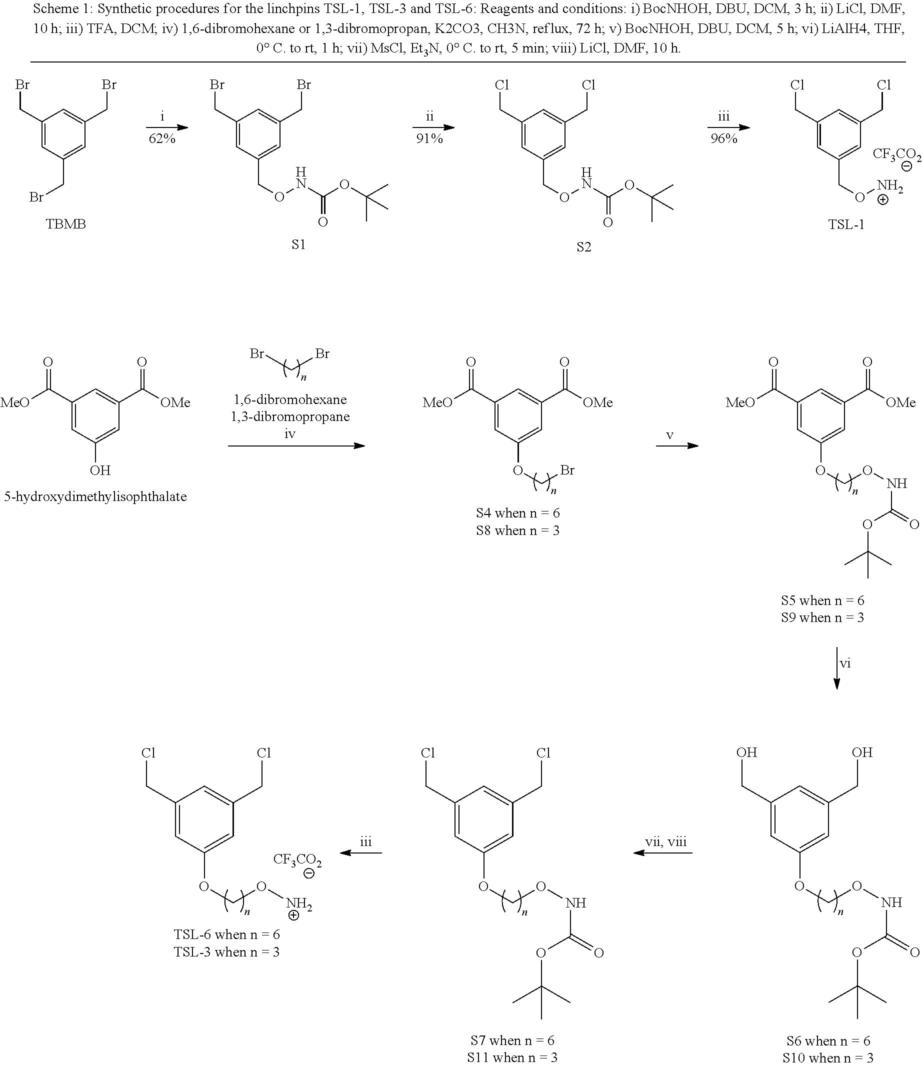

The chemical linker TSLC-7 containing amide and benzyl chloride functional groups was synthesized according to Scheme 2:

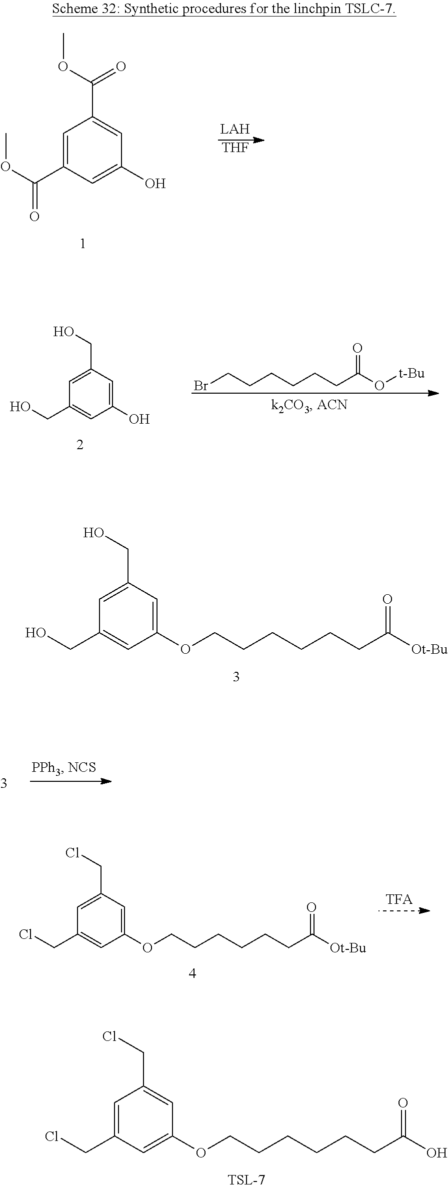

Synthetic procedures for the linchpins TSL-1, TSL-3, TSL-6 and TSLC-7:

### tert-Butyl ((3,5-bis(bromomethyl)benzyl)oxy)carbamate S1

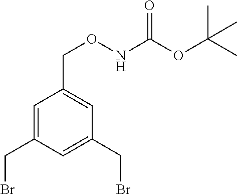 [SMILES: CC(C)(C)OC(=O)NOCc1cc(CBr)cc(CBr)c1]

To a solution of 1,3,5-Trisbromobenzylbenzene (TBMB) (2.5 g, 7 mmol) in DCM (30 mL), an equimolar solution of N-Boc-hydroxylamine (306 mg, 2.3 eq.) and DBU (0.3 mL, 2.3 eq.) in DCM (5 mL) was added over the course of 30 min and the resulting solution was stirred for 3 h. The solvent was removed on a rotary evaporator and the crude residue was purified over silica gel chromatography using ethyl acetate-hexanes (1:4) as eluent producing the title compound S1 as a white solid (707 mg, 25%): ¹H NMR (500 MHz, CDCₗ₃) δ=7.37 (s, 1H), 7.34 (s, 1H), 7.33 (s, 2H), 4.82 (s, 2H), 4.44 (s, 4H), 1.47 (s, 9H). ¹³C NMR (125 MHz, CDCl₃) δ=156.73, 138.65, 137.18, 129.59, 129.38, 81.89, 77.58, 32.48, 28.20. HRMS (ESI) calculated for C₁₄H₁₉Br₂NO₃Na [M+Na]+ m/z=429.9629, found 429.9226.

### tert-Butyl ((3,5-bis(chloromethyl)benzyl)oxy)carbamate S2

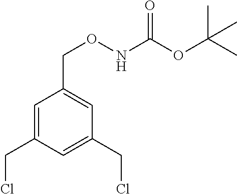 [SMILES: CC(C)(C)OC(=O)NOCc1cc(CCl)cc(CCl)c1]

Lithium chloride (196 mg, 3 eq.) was added to a solution of S1 (707 mg, 1.72 mmol) in DMF (10 mL) and the solution was stirred for 10 h. The reaction mixture was partitioned between ethyl acetate and water. The combined organic layers were washed with water and brine. The organic layer was dried over anhydrous sodium sulfate. After removing the solvent on a rotary evaporator, the crude residue was purified over silica gel chromatography using ethyl acetate-hexanes (1:4) as eluent producing the title compound S2 as colorless oil (457 mg, 83%). ¹H NMR (400 MHz, CDCₗ₃) δ=7.46 (s, 1H), 7.37 (s, 1H), 7.36 (s, 2H), 4.82 (s, 2H), 4.53 (s, 4H), 1.46 (s, 9H). ¹³C NMR (100 MHz, CDCl3) δ=156.99, 138.51, 137.29, 129.14, 128.86, 82.04, 77.85, 45.70, 28.39. HRMS (ESI) calculated for C₁₄H₁₉Cl₂NO₃Na [M+Na]+ m/z=342.0640, found 342.0632.

### O-(3,5-bis(chloromethyl)benzyl)hydroxylammonium 2,2,2-trifluoroacetate TSL-1

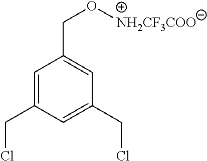

To a solution of S2 (450 mg, 1.4 mmol) in DCM (10 mL), TFA (0.5 mL, 5 eq.) was added and the mixture was stirred for 1 h. The volatiles were removed on a rotary evaporator. Residual TFA was azeotropically removed by repeatedly dissolving the resulting oil in toluene and evaporation on a rotary evaporator to produce the title compound TSL-1 as white viscous liquid (416 mg, 89%). To obtain product of higher purity 300 mg of crude TSL-1 was purified by semi preparative RP-HPLC and lyophilized to yield TSL-1 as light-yellow powder (212 mg, 71%). 1H NMR (400 MHz, CD3OD) δ=7.56 (s, 1H), 7.49 (d, 2H, J=1.6 Hz), 5.06 (s, 2H), 4.70 (s, 4H). 13C NMR (100 MHz, CD3OD) δ=141.5, 136.7, 131.7, 131.1, 78.3, 46.49. HRMS (ESI) calculated for C₉H₁₂Cl₂NO [M+H]+ m/z=220.0290, found 220.0289.

### Dimethyl 5-((6-bromohexyl)oxy)isophthalate S4

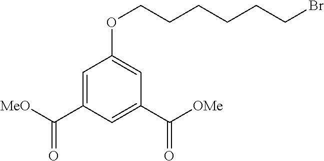 [SMILES: COC(=O)c1cc(OCCCCCCBr)cc(C(=O)OC)c1]

To a solution of 5-hydroxydimethylisophthalate (4.2 g, 20 mmol) and 1,6-dibromohexane (9.2 mL, 3 eq.) in CH₃CN (50 mL), potassium carbonate (8.3 g, 3 eq.) was added and the mixture was refluxed for 12 h. The reaction mixture was cooled to room temperature, diluted with water (50 mL) and extracted with ethyl acetate (3×20 mL). The combined organic layer was washed with water (50 mL) and brine (50 mL). Ethyl acetate was removed by rotary evaporator. Chromatography of the residue on silica gel using ethyl acetate-hexanes (7:1) as eluent produced the title compound S4 as white solid (5.3 g, 70%): ¹H NMR (500 MHz, CDCl3) δ=8.23 (s, 1H), 7.71 (s, 2H), 4.02 (t, 2H, J=6.5 Hz), 3.92 (s, 6H), 3.40 (t, 2H, J=6.5 Hz), 1.89-1.80 (m, 4H), 1.51-1.48 (m, 4H). ¹³C NMR (125 MHz, CDCl3) δ=166.43, 159.40, 132.00, 123.09, 120.07, 68.60, 33.95, 32.91, 29.18, 28.13, 25.48. HRMS (ESI) calculated for C₁₆H₂₁BrO₅Na [M+Na]+ m/z=395.0465, found 395.0472.

### Dimethyl 5-((6-(((tert-butoxycarbonyl)amino)oxy)hexyl)oxy)isophthalate S5

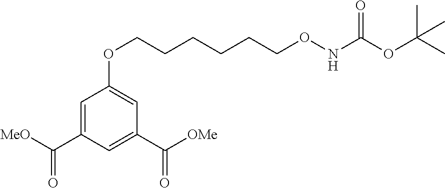 [SMILES: COC(=O)c1cc(OCCCCCCONC(=O)OC(C)(C)C)cc(C(=O)OC)c1]

A solution of lithium aluminum hydride (713 mg, 18.3 mmol) in THF (10 mL) was added to an ice cold solution of S7 (2.6 g, 6.1 mmol) in THF (25 mL) drop wise via cannula and the mixture was stirred for 3 h. Water was added very carefully until the evolution of hydrogen ceased. The white precipitate was filtered off and the solution was partitioned between ethyl acetate (3×30 mL) and water. The combined organic layers were washed with water, brine and dried over anhydrous sodium sulfate. Ethyl acetate was evaporated on a rotary evaporator and chromatography over silica gel of the crude residue with ethyl acetate-hexanes (1:1) produced the title compound S6 as a colorless gum (1.5 g, 66%): ¹H NMR (500 MHz, CDCₗ₃) δ=7.15 (s, 1H), 6.92 (s, 1H), 6.84 (s, 1H), 4.65 (s, 4H), 3.98 (t, 2H, J=6.5 Hz), 3.86 (t, 2H, J=6.5 Hz), 2.03 (bs, 2H), 1.82-1.76 (m, 2H), 1.69-1.64 (m, 2H), 1.52-1.43 (m, 13H). ¹³C NMR (125 MHz, CDCₗ₃) δ=159.61, 156.96, 142.81, 117.36, 112.17, 81.63, 76.70, 67.87, 65.13, 29.05, 28.23, 27.92, 25.83, 25.61. HRMS (ESI) calculated for C₁₉H₃₁NO₆Na [M+Na]+ m/z=392.2044, found 392.2046.

### tert-Butyl ((6-(3,5-bis(chloromethyl)phenoxy)hexyl)oxy)carbamate S7

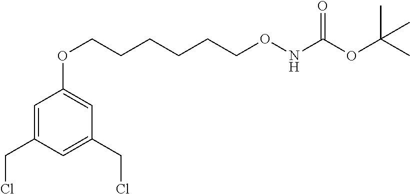 [SMILES: CC(C)(C)OC(=O)NOCCCCCCOc1cc(CCl)cc(CCl)c1]

To an ice cold solution of S6 (1.5 g, 4 mmol) and triethylamine (1.7 mL, 3 eq.) in DCM (20 mL), methane sulfonyl chloride (0.8 mL, 2.5 eq.) was added dropwise and the solution was stirred for 30 min. Without further purification THF (10 mL) and lithium chloride (500 mg, 3 eq.) was added subsequently. The ice bath was removed, and the reaction stirred for 12 h. The solvent was removed in a rotary evaporator and the crude residue was purified by chromatography over silica gel using ethyl acetate-hexanes (7:1) as eluent to produce the title compound S7 was as colorless oil (1.2 g, 74%). ¹H NMR (500 MHz, CDCₗ₃) δ=7.15 (s, 1H), 6.97 (s, 1H), 6.87 (s, 2H), 4.53 (s, 4H), 3.96 (t, 2H, J=6.5 Hz), 3.86 (t, 2H, J=6.5 Hz), 3.92 (s, 6H), 3.40 (t, 2H, J=7.0 Hz), 1.89-1.63 (m, 4H), 1.51-1.48 (m, 4H). ¹³C NMR (125 MHz, CDCₗ₃) δ=159.61, 156.91, 139.29, 120.69, 114.72, 81.60, 68.04, 52.57, 45.86, 29.02, 28.25, 27.97, 25.88, 25.69. HRMS (ESI) calculated for C₁₉H₂₉Cl₂NO₄Na [M+Na]+ m/z=428.1366, found 428.1372.

### O-(6-(3,5-bis(chloromethyl)phenoxy)hexyl)hydroxylammonium 2,2,2-trifluoroacetate TSL-6

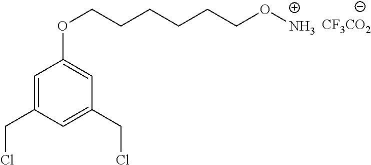

TFA (1.1 mL, 5 eq.) was added to a solution of S7 (1.2 g, 2.9 mmol) in DCM (15 mL) and stirred for 1 h. TFA and DCM was removed on a rotary evaporator. Residual TFA was azeotropically removed by repeatedly dissolving the resulting oil in toluene and evaporation on the rotary evaporator to produce the title compound TSL-6 as white viscous liquid (1.1 g, 89%). To yield a product of higher purity 100 mg of this compound was purified by RP-HPLC and lyophilized to produce the title compound TSL-6 as white powder (62 mg, 62%). ¹H NMR (500 MHz, CD3OD) δ=7.05 (s, 1H), 6.95 (s, 2H), 4.61 (s, 4H), 4.08-4.01 (m, 4H), 1.82-1.68 (m, 4H), 1.57-1.46 (m, 4H). ¹³C NMR (125 MHz, CD3OD) δ=161.81, 141.94, 122.82, 116.44, 77.12, 69.81, 47.29, 30.94, 29.48, 27.56, 27.23. HRMS (ESI) calculated for C₁₄H₂₂Cl₂NO₂ [M+H]+ m/z=306.1028, found 306.1026.

### Dimethyl 5-(3-bromopropoxy)isophthalate S8

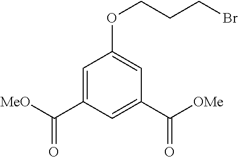 [SMILES: COC(=O)c1cc(OCCCBr)cc(C(=O)OC)c1]

To a solution of 5-hydroxydimethylisophthalate (900 mg, 2.3 mmol) and 1,3-dibromopropane (0.31 mL, 1.5 eq.) in CH₃CN (20 mL), potassium carbonate was added and the mixture was refluxed for 12 h. The reaction mixture was allowed to cool down to room temperature, diluted with water (60 mL) and extracted with ethyl acetate (3 □20 mL). The combined organic layer was washed with brine (50 mL). Ethyl acetate was removed on a rotary evaporator. Chromatography of the residue on silica gel in ethyl acetate-hexanes (7:1) produced the title compound S8 as white solid (980 mg, 85%): ¹H NMR (500 MHz, CDCl3) δ=8.27 (s, 1H), 7.74 (s, 2H), 4.18 (t, 2H, J=6.0 Hz), 3.92 (s, 6H), 3.60 (t, 2H, J=6.0 Hz), 2.33 (p, 2H, J=6.0 Hz). ¹³C NMR (125 MHz, CDCl3) δ=166.43, 159.13, 132.20, 123.56, 120.17, 66.23, 52.78, 32.48, 29.96. HRMS (ESI) calculated for C₁₃H₁₅BrO₅Na [M+Na]+ m/z=353.9995, found 353.0002.

### Dimethyl 5-(3-(((tert-butoxycarbonyl)amino)oxy)propoxy)isophthalate S9

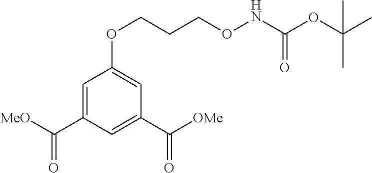 [SMILES: COC(=O)c1cc(OCCCONC(=O)OC(C)(C)C)cc(C(=O)OC)c1]

To a mixture of S8 (3 g, 9 mmol) and N-Boc hydroxylamine (1.4 g, 1.2 eq.) in DCM (20 mL), DBU (1.6 mL, 1.2 eq.) was added drop wise and the solution stirred for 5 h. DCM was evaporated on a rotary evaporator and the crude was subjected to chromatography over silica gel with ethyl acetate-hexanes (4:1) produced the title compound S9 as colorless oil (1.25 g, 36%). ¹H NMR (500 MHz, CDCl3) δ=8.26 (s, 1H), 7.74 (s, 2H), 7.21 (s, 1H) 4.18 (t, 2H, J=6.0 Hz), 4.04 (t, 2H, J=6.0 Hz), 3.93 (s, 6H), 2.14 (p, 2H, J=6.0 Hz), 1.47 (s, 9H). ¹³C NMR (125 MHz, CDCl3) δ=166.19, 159.03, 157.04, 131.81, 123.06, 119.91, 81.87, 73.14, 65.29, 52.44, 28.25, 28.05. HRMS (ESI) calculated for C₁₈H₂₅NO₈Na [M+Na]+ m/z=406.1472, found 406.1468.

### tert-Butyl (3-(3,5-bis(hydroxymethyl)phenoxy)propoxy)carbamate S10

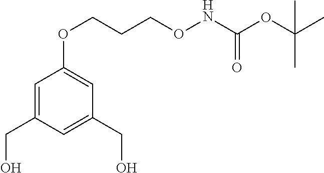 [SMILES: CC(C)(C)OC(=O)NOCCCOc1cc(CO)cc(CO)c1]

A solution lithium aluminium hydride (129 mg, 3 eq.) in THF (2 mL) was added to an ice cold solution of S9 (1.25 g, 3.2 mmol) in THF (10 mL) via cannula drop wise and the mixture was stirred for 3 h. Water was added carefully until the evolution of hydrogen ceased. The white precipitate was filtered off and the filtrate was extracted with ethyl acetate (3×30 mL). The combined organic layer was washed with water, brine and dried over anhydrous sodium sulfate. Ethyl acetate was evaporated on a rotary evaporator and purification of the crude residue by chromatography over silica gel with ethyl acetate-hexanes (1:1) as eluent produced the title compound S10 as a colorless gum (1.23 g, 86%). 1H NMR (500 MHz, CDCl3) δ=7.39 (s, 1H), 6.87 (s, 1H), 4.59 (s, 4H), 4.18 (t, 2H, J=6.0 Hz), 4.04 (t, 2H, J=6.0 Hz), 3.93 (s, 6H), 2.14 (t, 2H, J=6.5 Hz), 1.47 (s, 9H). 13C NMR (125 MHz, CDCl3) δ=159.63, 157.36, 143.14, 117.89, 112.48, 82.10, 73.61, 65.28, 65.01, 28.54, 28.42. HRMS (ESI) calculated for C₁₆H₂₅NO₆Na [M+Na]+ m/z=350.1574, found 350.1569.

### tert-Butyl (3-(3,5-bis(chloromethyl)phenoxy)propoxy)carbamate S11

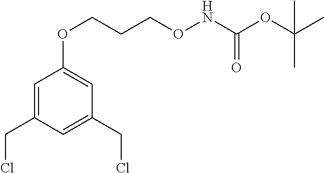 [SMILES: CC(C)(C)OC(=O)NOCCCOc1cc(CCl)cc(CCl)c1]

To an ice-cold solution of S10 (1.2 g, 3.7 mmol) and trimethylamine (1.5 mL, 3 eq.) in DCM (15 mL), methane sulfonyl chloride (0.7 mL, 2.5 eq.) was added dropwise and the solution was stirred for 30 minutes. Without further purification THF (5 mL) and lithium chloride (421 mg, 3 eq.) was added subsequently. The ice bath was removed and the reaction mixture was stirred for 12 h. The volatiles were evaporated on a rotary evaporator and the crude residue was subjected to chromatography over silica gel using ethyl acetate-hexanes (1:6) as eluent produced the title compound S11 as colorless oil (900 mg, 65%). 1H NMR (500 MHz, CDCl3) δ=6.99 (s, 1H), 6.90 (s, 2H), 4.54 (s, 4H), 4.31 (t, 2H, J=6.0 Hz), 4.13 (t, 2H, J=6.0 Hz), 2.15 (p, 2H, J=6.0 Hz), 1.48 (s, 9H). 13C NMR (125 MHz, CDCl3) δ=159.49, 151.69, 139.51, 121.14, 114.92, 86.19, 76.23, 45.97, 38.76, 28.27. HRMS (ESI) calculated for C₁₆H₂₃Cl₂NO₄Na [M+Na]+m/z=386.0896, found 386.0902.

### O-(3-(3,5-bis(chloromethyl)phenoxy)propyl)hydroxylammonium 2,2,2-trifluoroacetate TSL-3

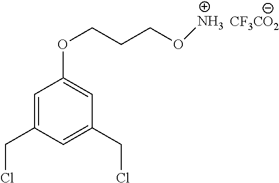

TFA (0.9 mL, 5 eq.) was added to a solution of S11 (900 mg, 2.4 mmol) in DCM (10 mL) and stirred for 1 h. TFA and DCM was removed on a rotary evaporator. Residual TFA was azeotropically removed by repeatedly dissolving the resulting oil in toluene and evaporation, which produced the title compound TSL-3 as white gummy liquid (725 mg, 80%). To obtain a product of higher purity, 300 mg of the title compound was purified in RP-HPLC and lyophilized to get a white powder (202 mg, 67%). ¹H NMR (400 MHz, CD3OD) δ=7.08 (s, 1H), 6.98 (s, 2H), 4.63 (s, 4H), 4.27 (t, 2H, J=6.0 Hz), 4.14 (t, 2H, J=6.0 Hz), 2.20 (p, 2H, J=6.0 Hz). ¹³C NMR (100 MHz, CD3OD) δ=161.3, 142.1, 123.3, 116.5, 73.9, 65.9, 29.7. HRMS (ESI) calculated for C₁₁H₁₆Cl₂NO₂ [M+Na]+ m/z=264.0553, found 264.0551.

It is understood that the skilled artisan may use any means of synthesis and purification necessary to obtain suitable linkers, including the linkers described herein, and that the synthesis scheme as described above may be modified in any way to obtain suitable linkers.

### Cyclization of Peptide Scaffolds to Form Compounds

A one pot peptide scaffold cyclization process was used to obtain the cyclic or bicyclic compounds as described herein.

Disulfide formation: synthetic, linear peptides described above were suspended in phosphate buffered solution: (10 mM NaH₂PO₄; 1.8 mM KH₂PO₄) plus 20% v/v DMSO to a final concentration of approximately 1 mg/mL and rocked at room temperature for 24 hours. The progress of oxidation was monitored by analytical C18 reverse-phase HPLC and the completion of the reaction was confirmed by LCMS.

Aldehyde formation: to the above, water containing 20% (v/v) of acetonitrile was added to a final concentration of 0.5 mM, followed by the addition of 1.2 equivalents of sodium periodate from a 500 mM stock solution and mixed and rocked at room temperature in the dark for five minutes. Quenching was performed using residual oxidizing agent with five equivalents of methionine from 125 mM stock solution, mixed and rocked at room temperature for 15 minutes.

Ligation of linker: 5% of TFA was added to the solution to adjust the pH to 3, and 1.5 equivalents of TSL-6 were added from a 30 mM stock solution, and rocked at room temperature for two hours. If solids were observed, 1-2 mL of acetonitrile was added to dissolve them. Reduction of the disulfide was done by adding five equivalents of TCEP from 125 mM stock solution and rocked at room temperature for 20 minutes.

Regarding the ligation of 3-chloropropanoic acid, 2-(chloromethyl)benzoic acid, DIG, and chloroacetic acid linkers, incorporation of patent no. U.S. Pat. No. 9,809,623 B2 with regards to acylating organic compounds is made herein.

Bicyclization was performed by adjusting the pH to 10 with carbonate-bicarbonate buffer at 500 nM (about 20% (v/v) of the volume used in aldehyde formation and left at room temperature overnight.

The crude reaction was purified on a C18 reverse phase HPLC column (Symmetry, 5 μm particle size, 19×50 mm, 100 Å pore size). The desired product was eluted with a linear gradient from 2% to 75% buffer B in 40 min. Flow rate 8 mL/min. Buffer A: H₂O+0.1% (v/v) TFA. Buffer B: Acetonitrile+0.1% (v/v) TFA. Formation and purity of the bicycle product was confirmed by LCMS.

It is understood that the skilled artisan may make suitable modifications to the above cyclization procedure to obtain suitable compounds.

### Representative Compounds

Table 3 describes representative compounds made according to the present disclosure.

TABLE 3
Representative compounds

| | | IC50 | |
|---|---|---|---|
| No. | | (μM) | Structure |
| 1 | Sulfide- BzR(NMe)SDTL (Pen)WE | 0.003 | 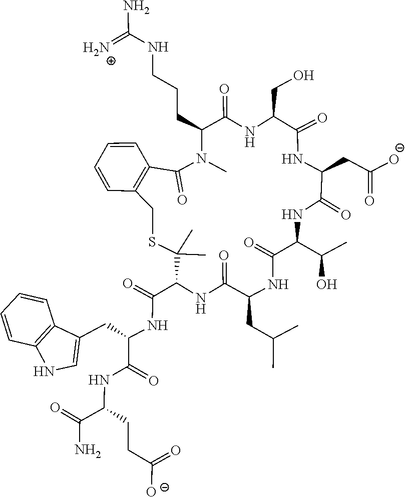 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)c2ccccc2CSC(C)(C)[C@@H](C(=O)N[C@@H](Cc2c[nH]c3ccccc23)C(=O)N[C@H](CCC(=O)[O-])C(N)=O)NC1=O] |
| 2 | Sulfide- ProR(NMe) SDTLCW | 0.033 | 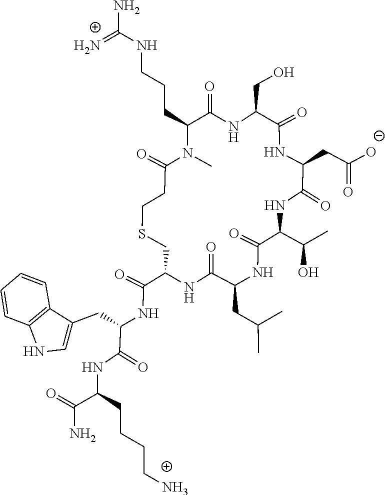 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)CCSC[C@@H](C(=O)N[C@@H](Cc2c[nH]c3ccccc23)C(=O)N[C@@H](CCCC[NH3+])C(N)=O)NC1=O] |
| 3 | TSL6- SRCP{Nle} DVWQEC | 0.053 | 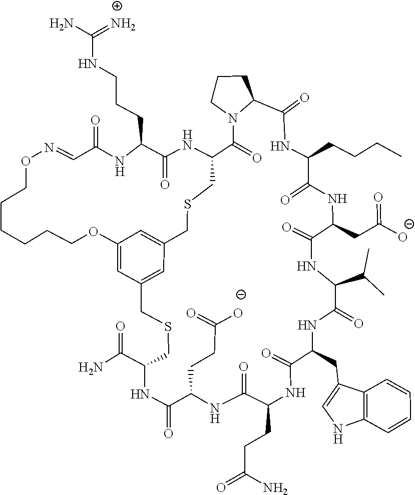 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 4 | TSLC7- GRCP{Nle} DIWQEC | 0.075 | 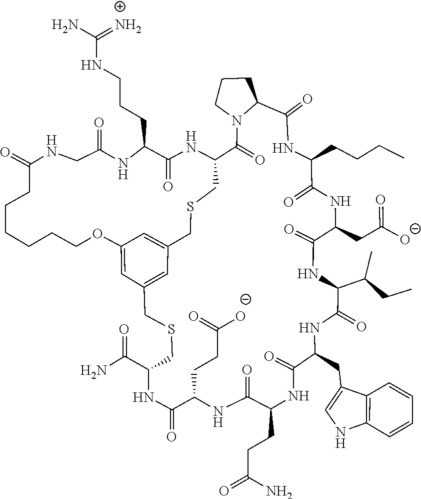 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 5 | Disulfide-Ac- CRSDTLC | 0.122 | 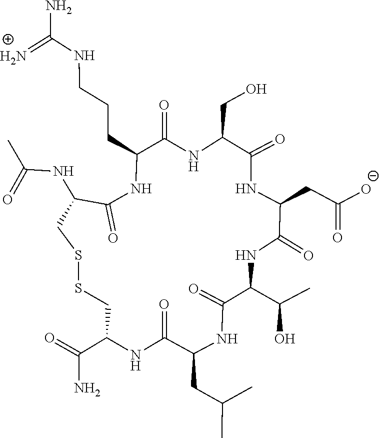 [SMILES: CC(=O)N[C@H]1CSSC[C@@H](C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 6 | TSL6- SRCP{Nle} DVWYEC | 0.130 | 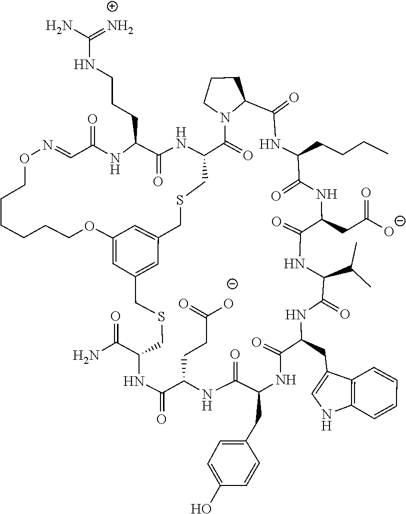 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC(=O)[C@H](C(C)C)NC(=O)[C@H](CC(=O)[O-])NC1=O] |
| 7 | Disulfide-Ac- CRSDTLCGE | 0.136 | 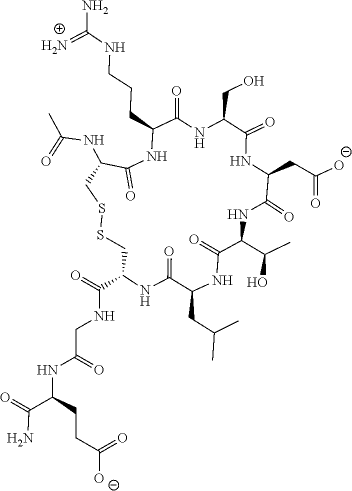 [SMILES: CC(=O)N[C@H]1CSSC[C@@H](C(=O)NCC(=O)N[C@@H](CCC(=O)[O-])C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 8 | Disulfide- CRSDTLC | 0.186 | 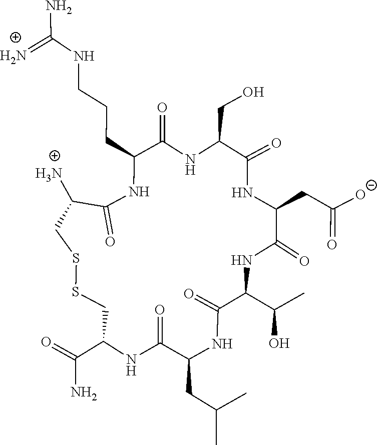 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]([NH3+])CSSC[C@@H](C(N)=O)NC1=O] |
| 9 | TSL6- SRCP{Nle} DIWQEC | 0.187 | 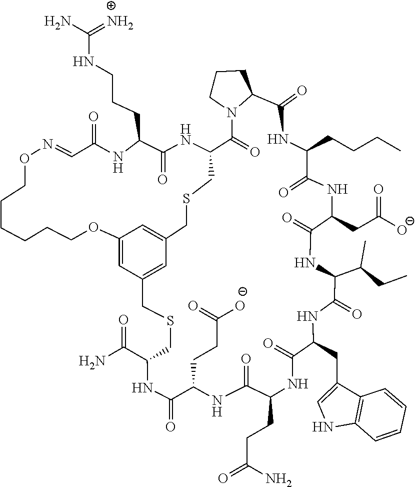 |
| 10 | TSL6- SKCPLDVPYEC | 0.188 | 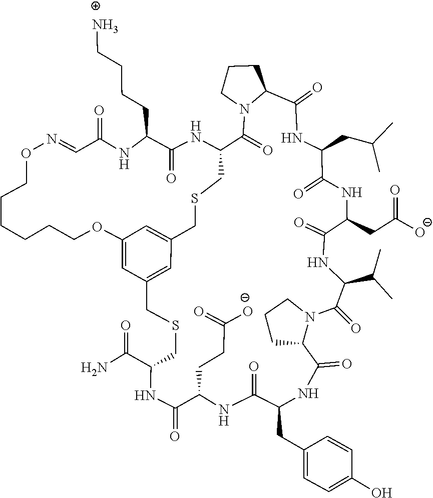 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCCN)C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C(C)C)NC(=O)[C@H](CC(=O)[O-])NC1=O] |
| 11 | TSL6- SKCPLDIPYEC | 0.198 | 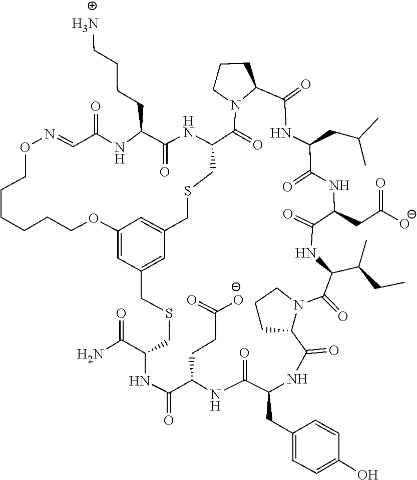 [SMILES: CC[C@H](C)[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCCN)C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 12 | TSL6- SRCP{Nle} DIAAEC | 0.204 | 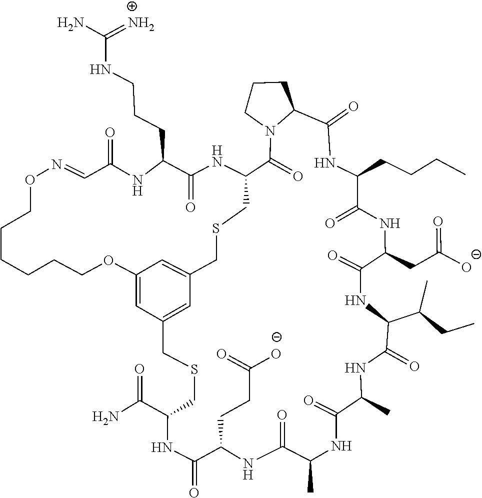 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](C)NC(=O)[C@H](C)NC(=O)[C@H]([C@@H](C)CC)NC(=O)[C@H](CC(=O)[O-])NC1=O] |
| 13 | TSL6- SRCPMDIAGEC | 0.227 | 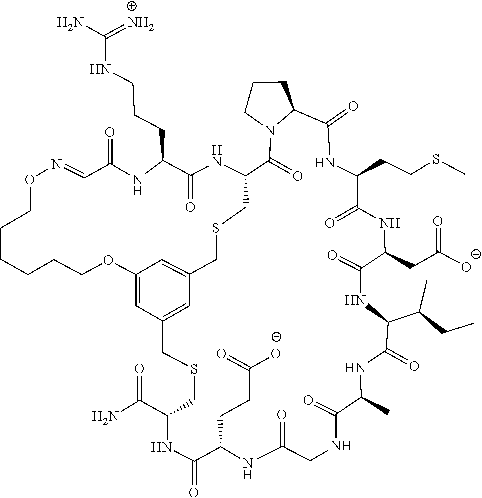 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC1=O] |
| 14 | TSL6- SRCP{Hse (OMe)} DIWQEC | 0.260 | 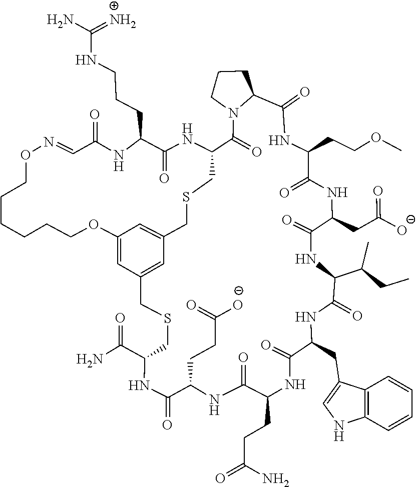 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 15 | TSL6- SRCP{Nle} DVAGEC | 0.261 | 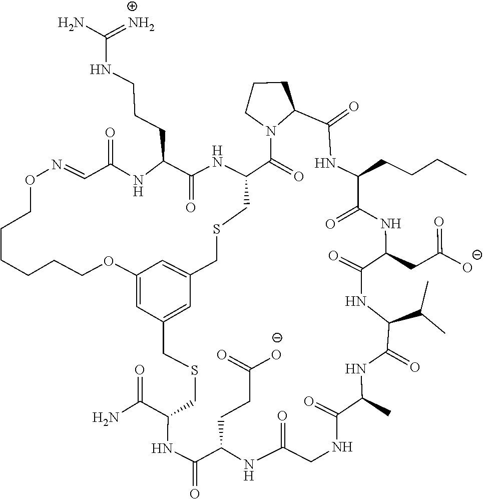 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 16 | TSL6- SRCPMDIWQEC | 0.272 | 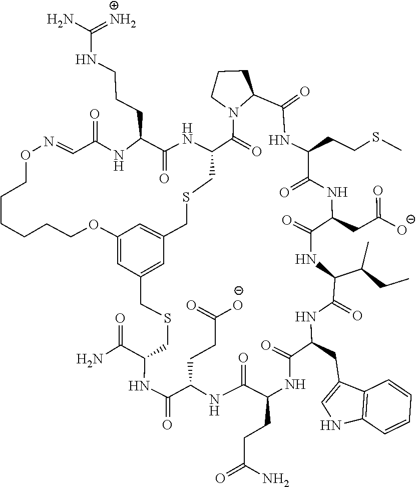 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 17 | TSL6- SRCPLDTPYEC | 0.307 | 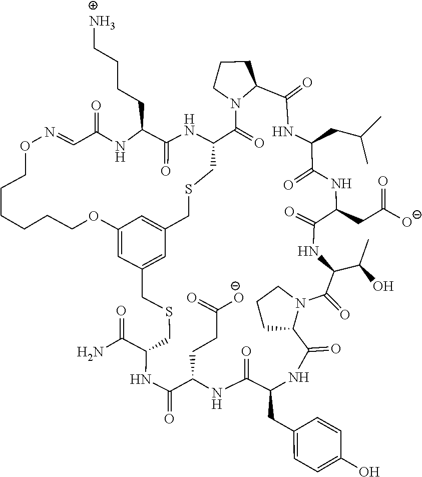 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 18 | TSL6- SKCPMDIPTC | 0.345 | 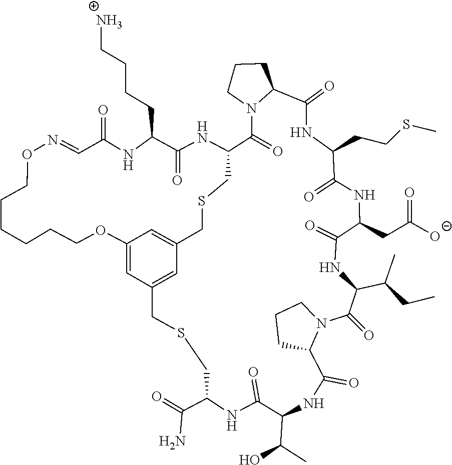 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H]([C@@H](C)O)NC(=O)[C@@H]2CCCN2C1=O] |
| 19 | TSL6- SKCPLDLPYEC | 0.363 | 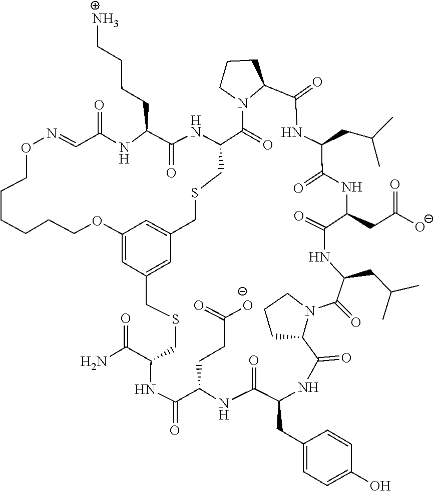 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](CC(C)C)NC(=O)[C@H](CC(=O)[O-])NC1=O] |
| 20 | TSL6- SRCPMDIPTC | 0.404 | 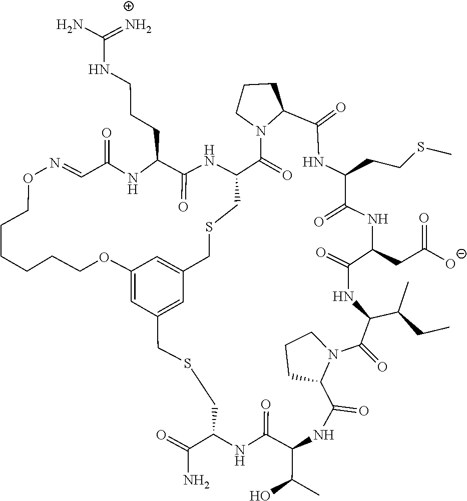 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H]([C@@H](C)O)NC(=O)[C@@H]2CCCN2C1=O] |
| 21 | TSLC7- GKCP{Nle} DIWVKC | 0.450 | 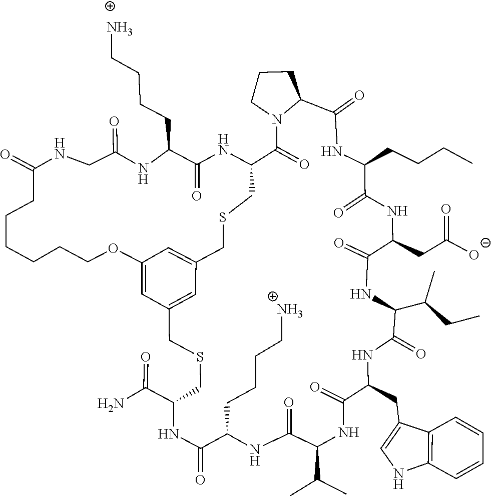 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCCN)C(=O)N2)CSC[C@@H](C(N)=O)NC(=O)[C@H](CCCC[NH3+])NC(=O)[C@H](C(C)C)NC(=O)[C@H](Cc2c[nH]c3ccccc23)NC(=O)[C@H]([C@@H](C)CC)NC(=O)[C@H](CC(=O)[O-])NC1=O] |
| 22 | TSL6- SKCPLDTPYEC | 0.495/0.94 | 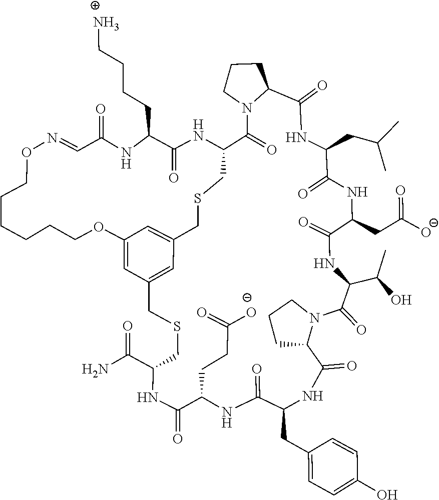 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 23 | TSL6- SRCPMDVAGEC | 0.511 | 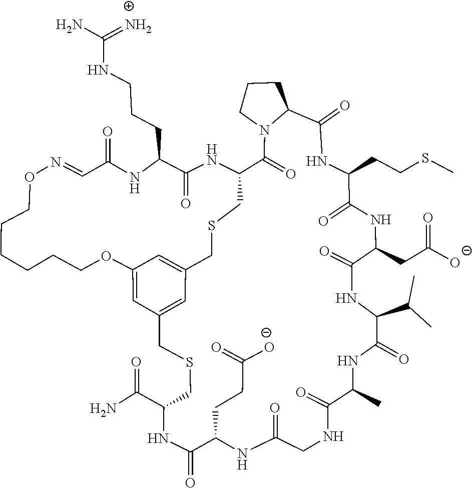 [SMILES: CSCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 24 | TSL6- SKCPLDTPYAC | 0.517 | 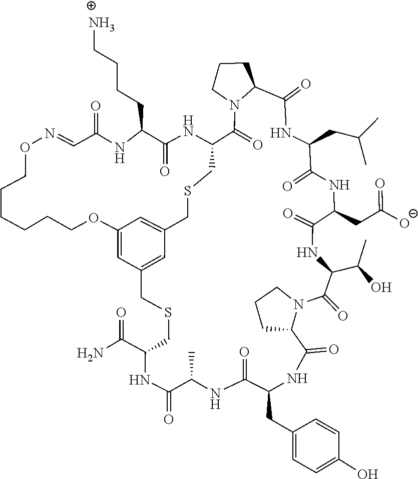 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 25 | Disulfide- SKCPLDTPYEC | 0.552 | 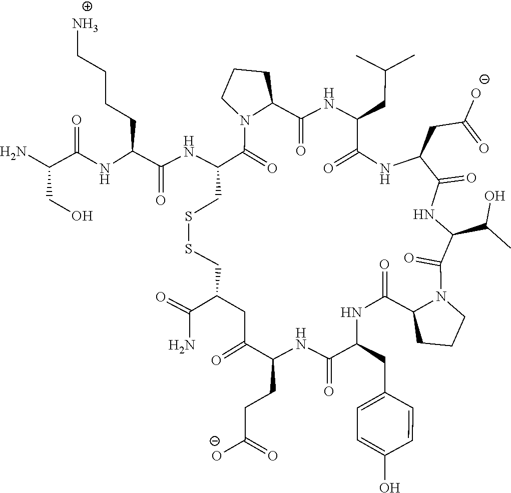 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)CC(=O)[C@H](CCC(=O)[O-])NC(=O)[C@H](Cc2ccc(O)cc2)NC(=O)[C@@H]2CCCN2C(=O)[C@H](C(C)O)NC(=O)[C@H](CC(=O)[O-])NC1=O] |
| 26 | TSL6- SKCPLDTPQEC | 0.602 | 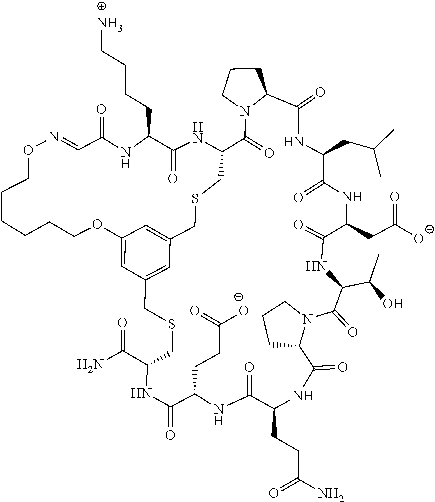 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](CCC(N)=O)NC(=O)[C@@H]2CCCN2C(=O)[C@H]([C@@H](C)O)NC(=O)[C@H](CC(=O)[O-])NC1=O] |
| 27 | TSL6- SRCP{Nle} DIWVKC | 0.620 | 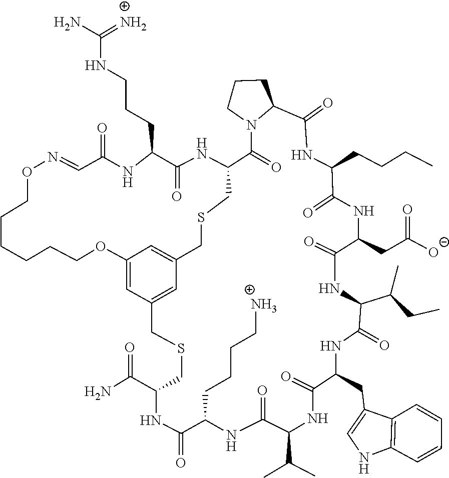 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCCC[NH3+])NC(=O)[C@H](C(C)C)NC(=O)[C@H](Cc2c[nH]c3ccccc23)NC(=O)[C@H]([C@@H](C)CC)NC(=O)[C@H](CC(=O)[O-])NC1=O] |
| 28 | TSL6- SQCPMDVPTC | 0.672 | 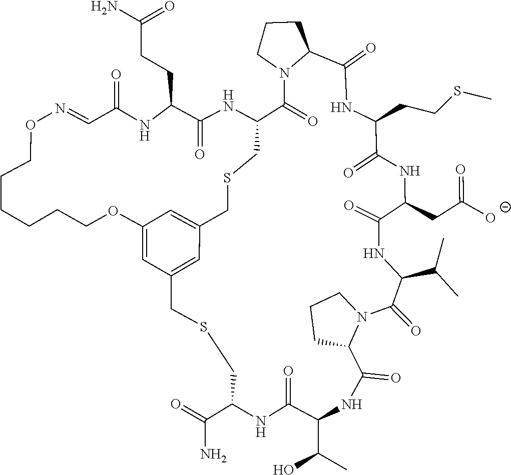 [SMILES: CSCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C(=O)[C@H](C(C)C)NC(=O)[C@H](CC(=O)[O-])NC1=O] |
| 29 | TSL6- SKCPLDMPYEC | 0.672 | 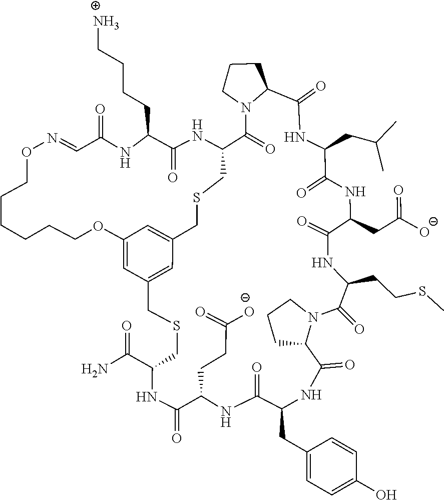 [SMILES: CSCC[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 30 | TSL6- SRCP{tBuAla} DVPQEC | 0.690 | 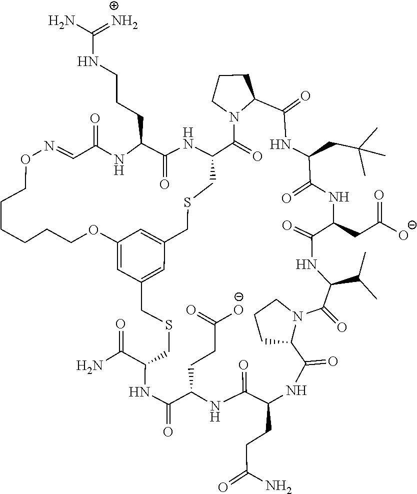 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@@H]2CCCN2C1=O] |
| 31 | TSL6- SKCPLDVPSC | 0.723 | 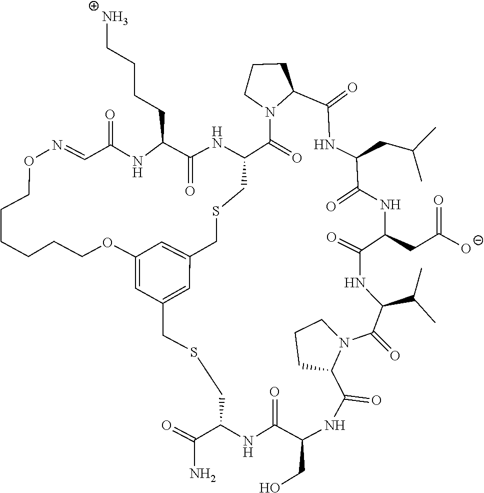 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 32 | DIG dimer-Ac- SRCPMDIAGECK | 0.729 | 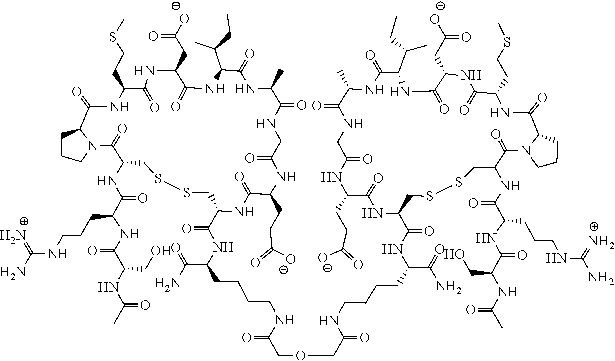 [SMILES: CC[C@H](C)[C@@H]1NC(=O)[C@H](CC(=O)[O-])NC(=O)[C@H](CCSC)NC(=O)[C@@H]2CCCN2C(=O)C(NC(=O)[C@H](CCCNC(N)=[NH2+])NC(=O)[C@H](CO)NC(C)=O)CSSC[C@@H](C(=O)N[C@@H](CCCCNC(=O)COCC(=O)NCCCC[C@H](NC(=O)[C@@H]2CSSC[C@H](NC(=O)[C@H](CCCNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)NC(=O)[C@H](CCC(=O)[O-])NC(=O)CNC(=O)[C@H](C)NC1=O] |
| 33 | TSLC7- GACP{Nle} DVWQEC | 0.740 | 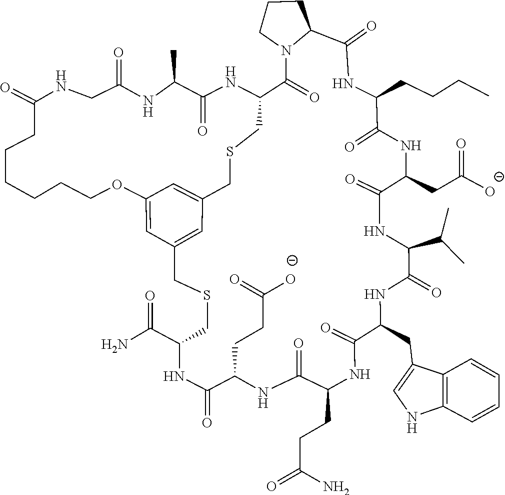 [SMILES: CCCC[C@@H]1NC(=O)[C@@H]2CCCN2C(=O)[C@@H]2CSCc3cc(cc(c3)OCCCCCCC(=O)NCC(=O)N[C@@H](C)C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 34 | TSL6- SKCPLDTPTEC | 0.748 | 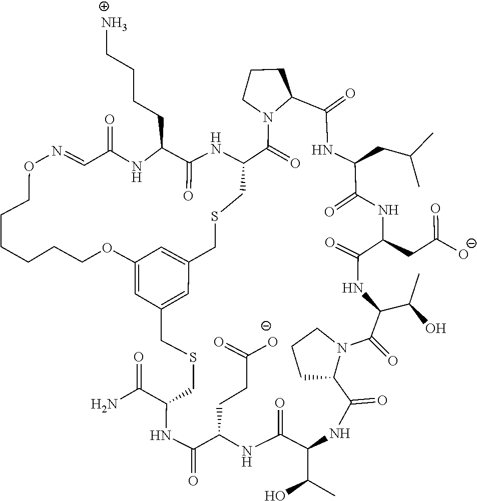 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H]([C@@H](C)O)NC(=O)[C@@H]2CCCN2C(=O)[C@H]([C@@H](C)O)NC(=O)[C@H](CC(=O)[O-])NC1=O] |
| 35 | TSL6- SKCPLDTPAEC | 0.796 | 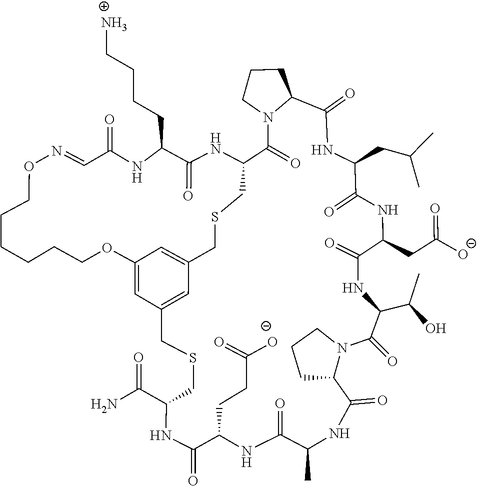 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](C)NC(=O)[C@@H]2CCCN2C(=O)[C@H]([C@@H](C)O)NC(=O)[C@H](CC(=O)[O-])NC1=O] |
| 36 | Disulfide-Ac- SRCPMDIAGEC | 0.932 | 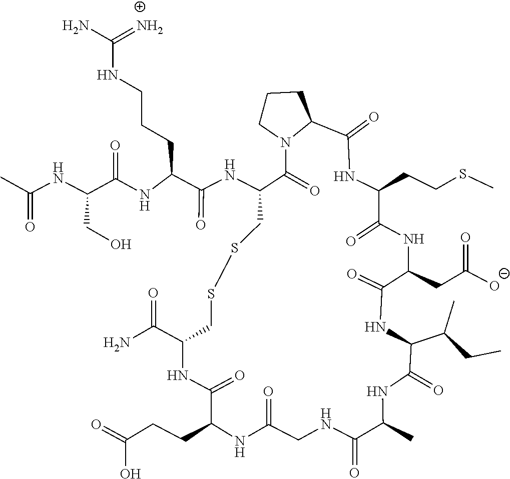 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(N)=O)NC(=O)[C@H](CCC(=O)O)NC(=O)CNC(=O)[C@H](C)NC1=O] |
| 37 | TSL6- SACPMDIPTC | 0.943 | 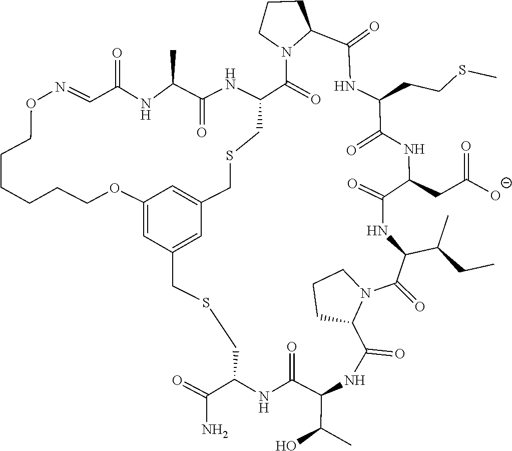 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H]([C@@H](C)O)NC(=O)[C@@H]2CCCN2C1=O] |
| 38 | TSL6- SKCPLDTAYEC | 1.042 | 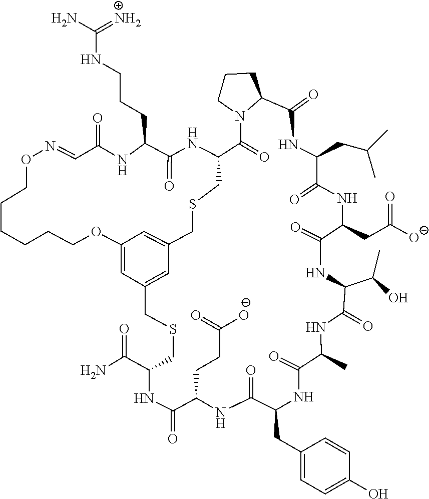 |
| 39 | TSL6- SRCP{Nle}D {tBuAla}PYEC | 1.13 | 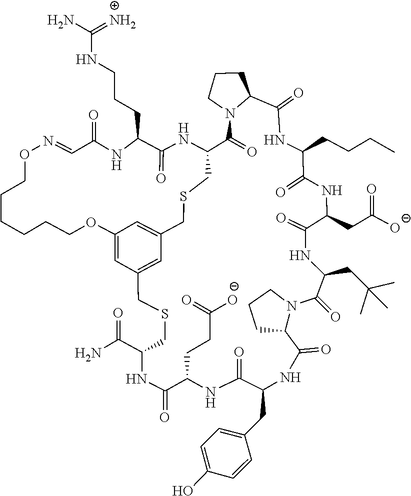 |
| 40 | TSL6- SQCPMDIPTC | 1.27 | 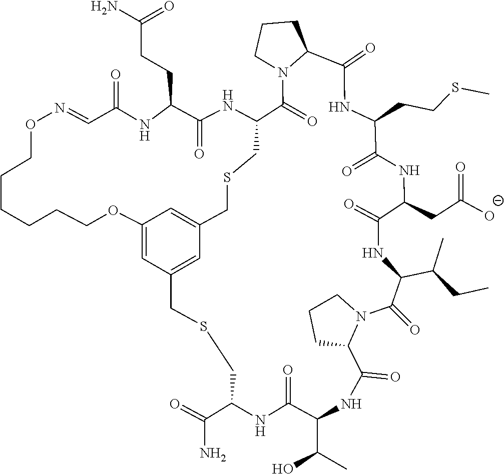 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 41 | Disulfide- SKCPLDVPSC | 1.299 | 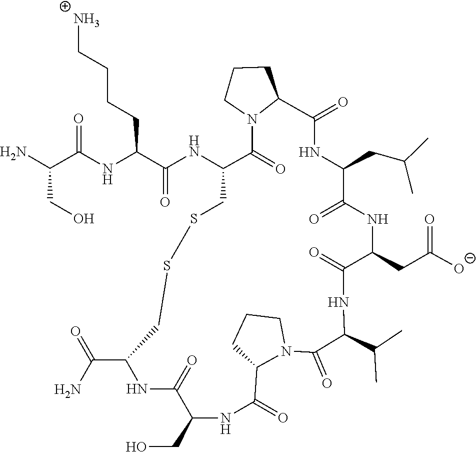 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 42 | TSL6- SKCPLDAPYEC | 1.40 | 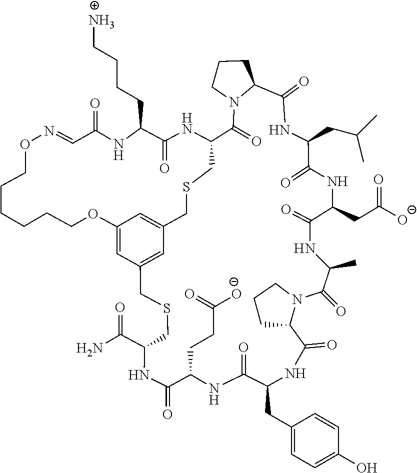 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C)NC(=O)[C@H](CC(=O)[O-])NC1=O] |
| 43 | TSL6- SACPLDTPYEC | 1.45 | 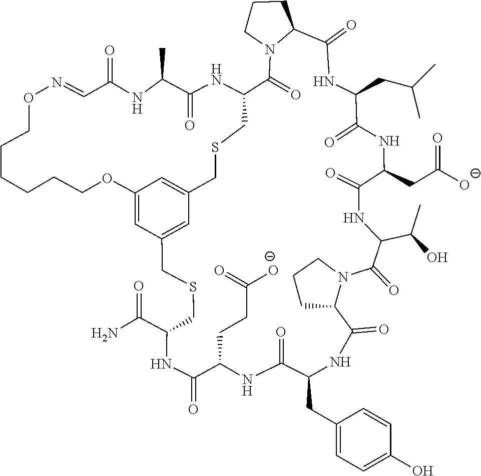 [SMILES: CC(C)C[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)C([C@@H](C)O)NC(=O)[C@H](CC(=O)[O-])NC1=O] |
| 44 | TSL6- SDCPDAERYIC | 1.463 | 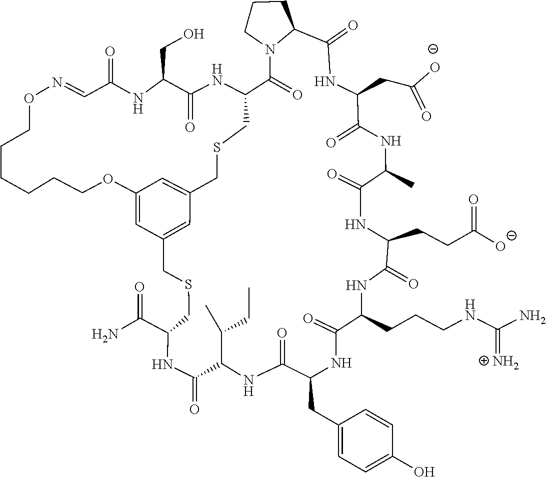 |
| 45 | TSLC7- GKCP{Nle}D {CbA}WQEC | 1.614 | 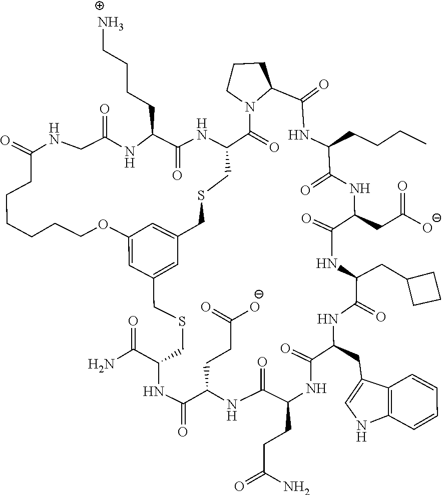 |
| 46 | TSL6- SQCPMDIPAC | 1.641 | 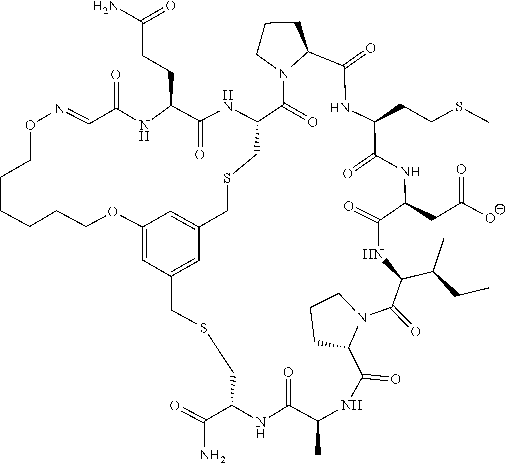 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H](C)NC(=O)[C@@H]2CCCN2C1=O] |
| 47 | Disulfide-Ac- SQCPMDIPTC | 1.696 | 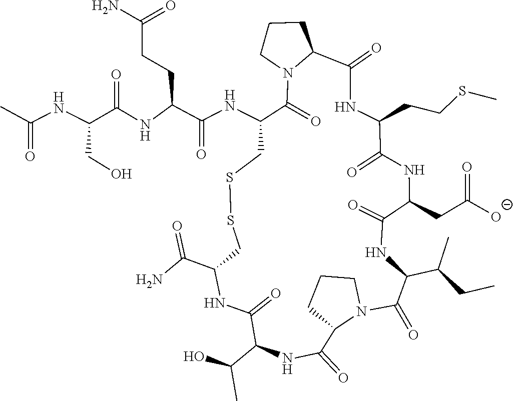 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCC(N)=O)NC(=O)[C@H](CO)NC(C)=O)CSSC[C@@H](C(N)=O)NC(=O)[C@H]([C@@H](C)O)NC(=O)[C@@H]2CCCN2C1=O] |
| 48 | TSL6- SRCP{Hse(OMe)} DIWYEC | 1.71 | 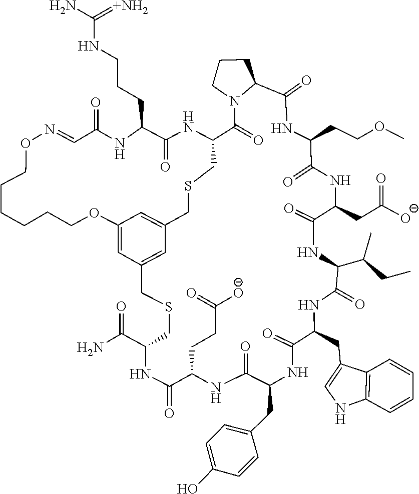 |
| 49 | BisPEG5-Ac- SRCPMDIAGECK | 1.731 | 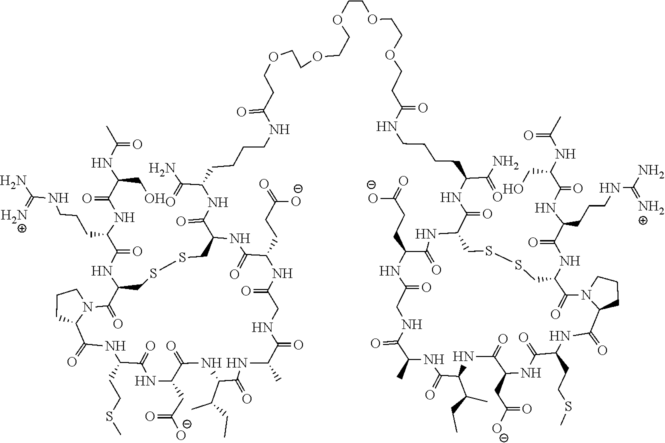 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(=O)N[C@@H](CCCCNC(=O)CCOCCOCCOCCOCCOCCC(=O)NCCCC[C@H](NC(=O)[C@@H]2CSSC[C@H](NC(=O)[C@H](CCCNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)NC(=O)[C@H](CCC(=O)[O-])NC(=O)CNC(=O)[C@H](C)NC1=O] |
| 50 | TSL6- SKCPLDAPSC | 1.74 | 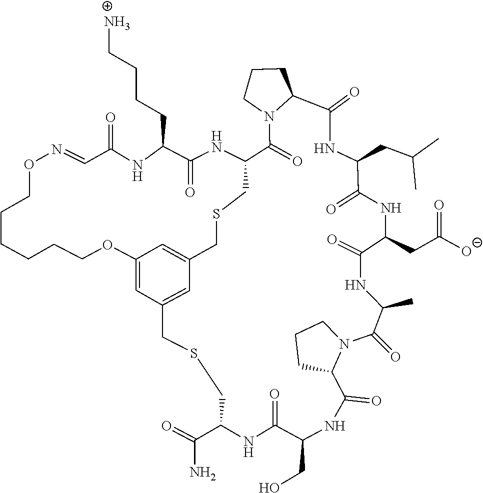 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C)NC(=O)[C@H](CC(=O)[O-])NC1=O] |
| 51 | TSL6- SKCPLDVPAC | 1.922 | 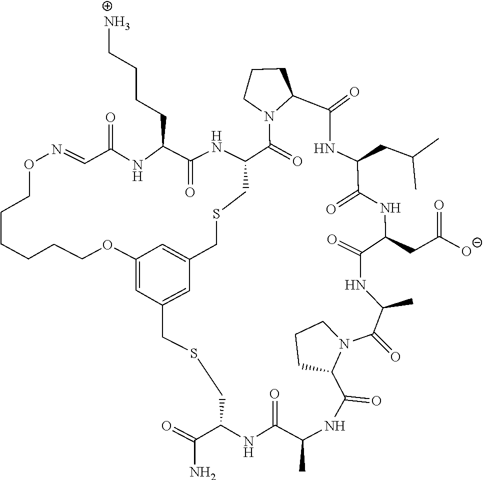 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@@H]2CCCN2C(=O)[C@H](C)NC(=O)[C@H](CC(=O)[O-])NC1=O] |
| 52 | TSL6- SQCPMDAPTC | 2.486 | 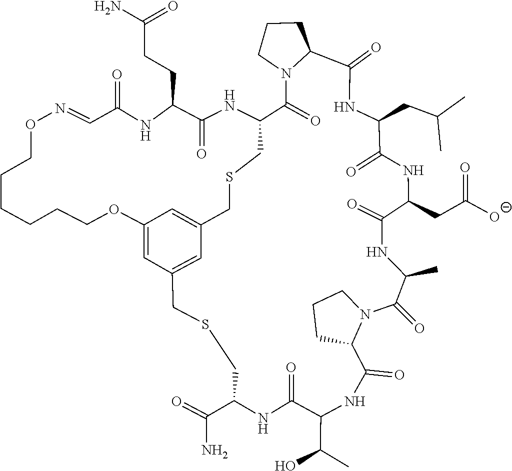 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)C([C@@H](C)O)NC(=O)[C@@H]2CCCN2C(=O)[C@H](C)NC(=O)[C@H](CC(=O)[O-])NC1=O] |
| 53 | TSL6- SKCALDTPYEC | 2.7 | 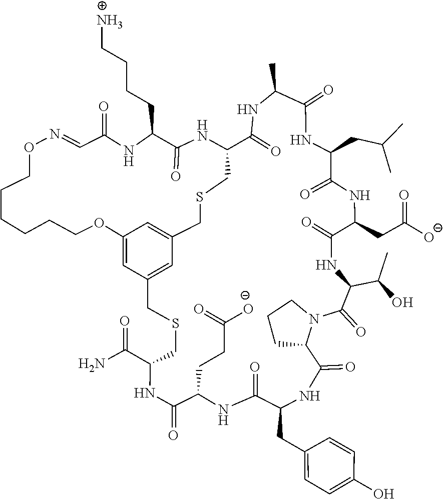 [SMILES: CC(C)C[C@@H]1NC(=O)[C@H](C)NC(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 54 | TSL6- SACPMDVWQEC | 3.063 | 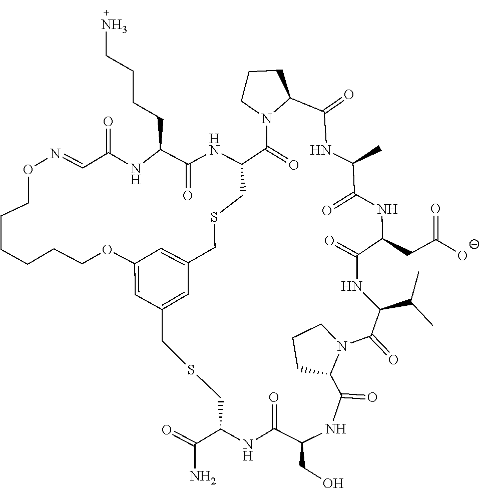 [SMILES: CC(C)[C@@H]1NC(=O)[C@H](CC(=O)[O-])NC(=O)[C@H](C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C1=O] |
| 55 | TSL6- SRCP{Nle}D {CbA}WQEC | 3.45 | 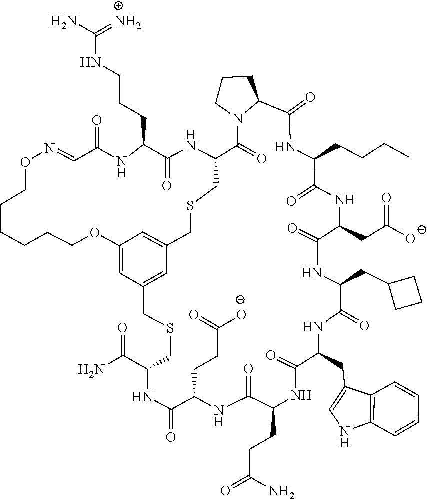 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](CC2CCC2)NC(=O)[C@H](CC(=O)[O-])NC1=O] |
| 56 | TSL6- SRCP{CbA}D {tBuAla}WQEC | 3.89 | 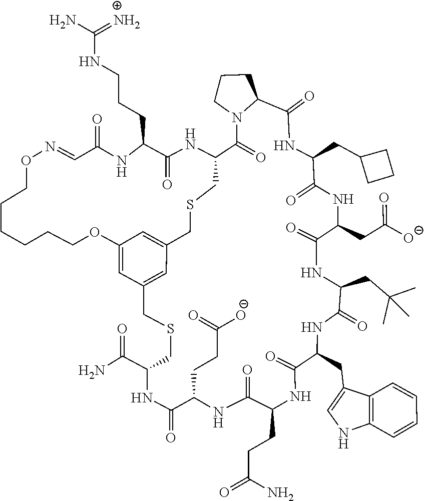 [SMILES: CC(C)(C)C[C@@H]1NC(=O)[C@H](CC(=O)[O-])NC(=O)[C@H](CC2CCC2)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 57 | TSL6- SACAWHQHAC | 3.895 | 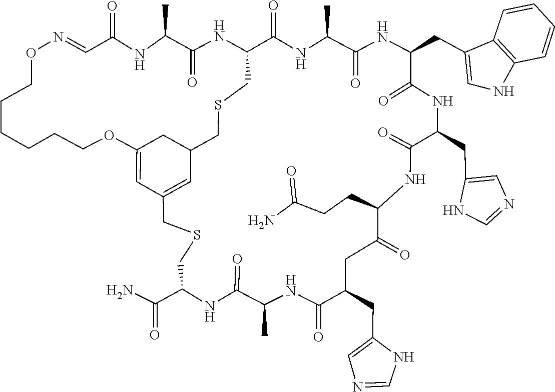 [SMILES: C[C@@H]1NC(=O)/C=N/OCCCCCCOC2=CC3=CC(CSC[C@H](NC1=O)C(=O)N[C@@H](C)C(=O)N[C@@H](Cc1c[nH]c4ccccc14)C(=O)N[C@@H](Cc1cnc[nH]1)C(=O)N[C@H](CCC(N)=O)C(=O)C[C@@H](Cc1cnc[nH]1)C(=O)N[C@@H](C)C(=O)N[C@H](C(N)=O)CSC3)C2] |
| 58 | TSL6- SRCP{tBuAla} DVWGEC | 4.01 | 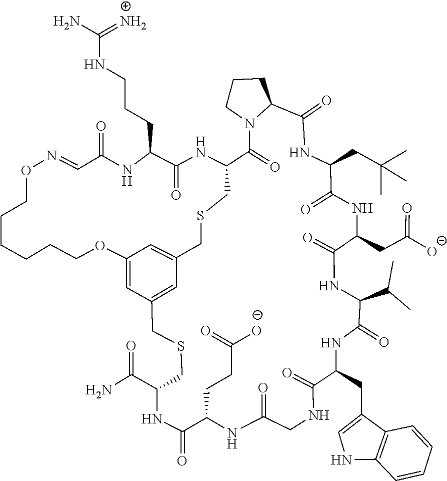 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](Cc2c[nH]c3ccccc23)NC1=O] |
| 59 | TSL6- SKCALDVPSC | 4.015 | 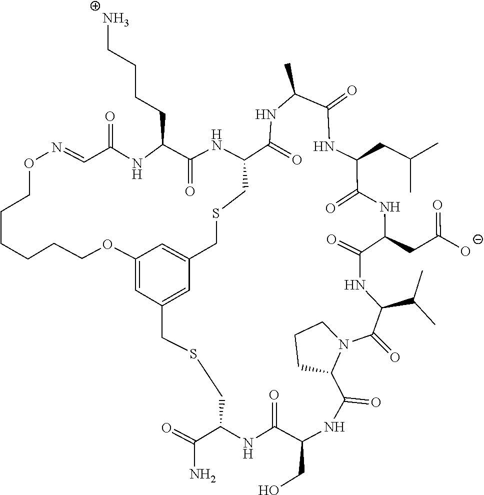 [SMILES: CC(C)C[C@@H]1NC(=O)[C@H](C)NC(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 60 | TSL6- SDCHDKFTEC | 5.432 | 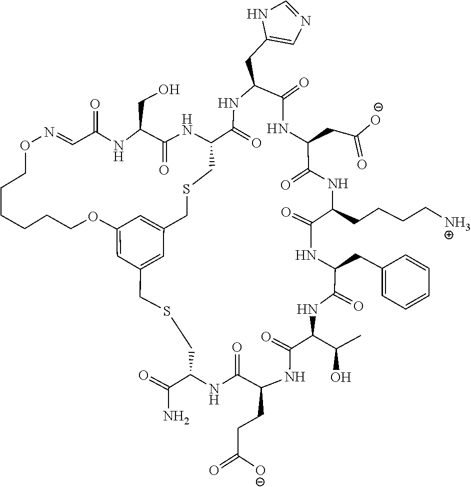 [SMILES: C[C@@H](O)[C@@H]1NC(=O)[C@H](Cc2ccccc2)NC(=O)[C@H](CCCC[NH3+])NC(=O)[C@H](CC(=O)[O-])NC(=O)[C@H](Cc2cnc[nH]2)NC(=O)[C@@H]2CSCc3cc(cc(c3)OCCCCCCO/N=C/C(=O)N[C@@H](CO)C(=O)N2)CSC[C@@H](C(N)=O)NC(=O)[C@H](CCC(=O)[O-])NC1=O] |
| 61 | TSL6- SACPMDVAGEC | 5.981 | 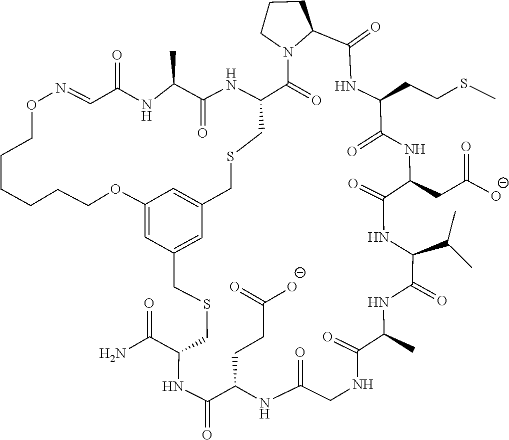 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 62 | TSL6- SDCHDPPKVC | 6.419 | 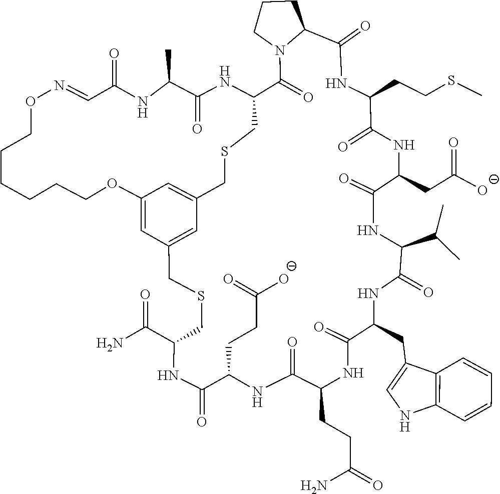 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 63 | TSL6- SLCYEWEHAC | 7.95 | 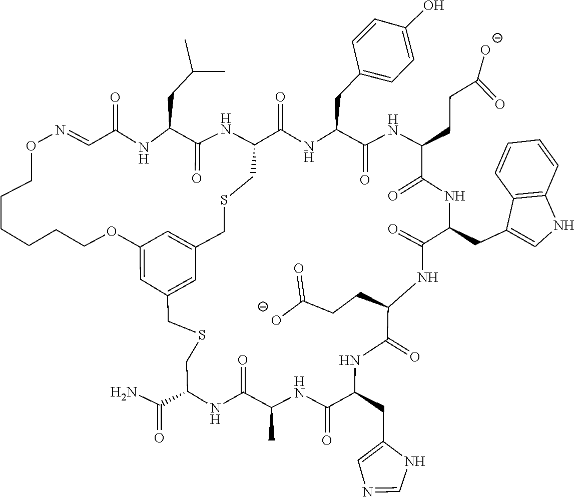 [SMILES: CC(C)C[C@@H]1NC(=O)/C=N/OCCCCCCOc2cc3cc(c2)CSC[C@H](NC1=O)C(=O)N[C@@H](Cc1ccc(O)cc1)C(=O)N[C@@H](CCC(=O)[O-])C(=O)N[C@@H](Cc1c[nH]c2ccccc12)C(=O)N[C@H](CCC(=O)[O-])C(=O)N[C@@H](Cc1cnc[nH]1)C(=O)N[C@@H](C)C(=O)N[C@H](C(N)=O)CSC3] |
| 64 | TSL6- SQCP{Nle}DIATC | 10.7 | 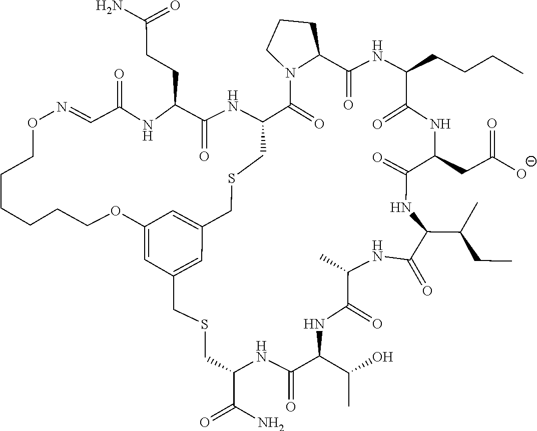 [SMILES: CCCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@H](C)NC(=O)[C@H]([C@@H](C)CC)NC(=O)[C@H](CC(=O)[O-])NC1=O] |
| 65 | TSL6- SDCPDSMAHSC | 13.81 | 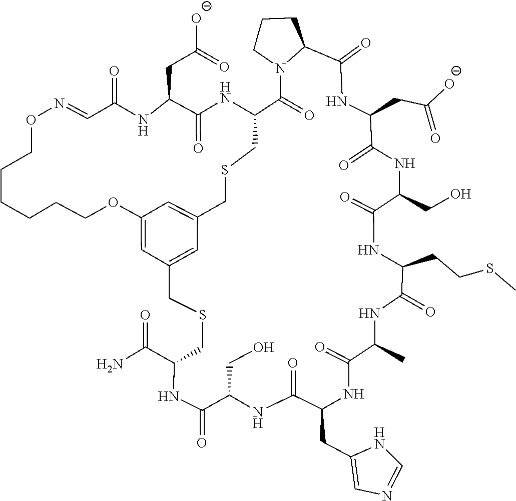 [SMILES: CSCC[C@@H]1NC(=O)[C@H](CO)NC(=O)[C@H](CC(=O)[O-])NC(=O)[C@@H]2CCCN2C(=O)[C@@H]2CSCc3cc(cc(c3)OCCCCCCO/N=C/C(=O)N[C@@H](CC(=O)[O-])C(=O)N2)CSC[C@@H](C(N)=O)NC(=O)[C@H](CO)NC(=O)[C@H](Cc2cnc[nH]2)NC(=O)[C@H](C)NC1=O] |
| 66 | TSL6- SICRTDVPIDC | 14.42 | 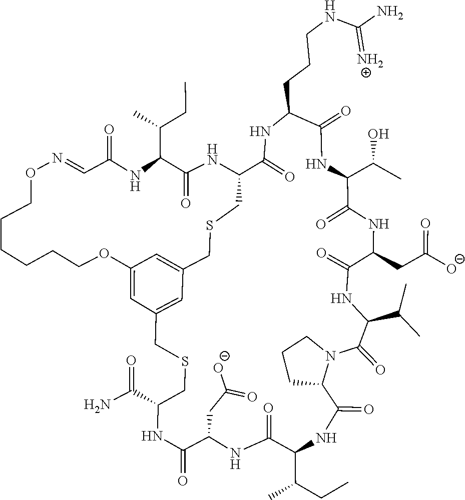 [SMILES: CC[C@@H](C)[C@@H]1NC(=O)/C=N/OCCCCCCOc2cc3cc(c2)CSC[C@H](NC1=O)C(=O)N[C@@H](CCCNC(N)=[NH2+])C(=O)N[C@@H]([C@@H](C)O)C(=O)N[C@@H](CC(=O)[O-])C(=O)N[C@@H](C(C)C)C(=O)N1CCC[C@H]1C(=O)N[C@@H]([C@@H](C)CC)C(=O)N[C@@H](CC(=O)[O-])C(=O)N[C@H](C(N)=O)CSC3] |
| 67 | TSL6- SQCP{Ox- Met}DAPTC | 15.7 | 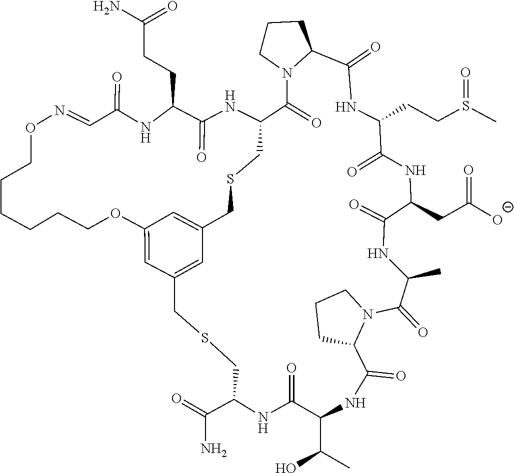 |
| 68 | Disulfide- RCSDTLCRS {NIe}DICWQ | 15.9 | 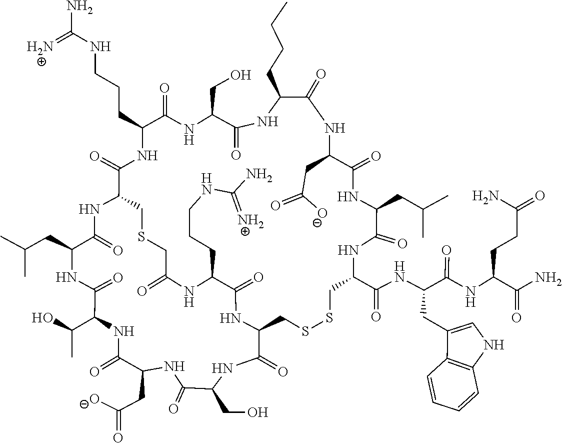 [SMILES: CCCC[C@@H]1NC(=O)[C@H](CO)NC(=O)[C@H](CCCNC(N)=[NH2+])NC(=O)[C@@H]2CSCC(=O)N[C@@H](CCCNC(N)=[NH2+])C(=O)N[C@@H](CSSC[C@@H](C(=O)N[C@@H](Cc3c[nH]c4ccccc34)C(=O)N[C@@H](CCC(N)=O)C(N)=O)NC(=O)[C@H](CC(C)C)NC(=O)[C@@H](CC(=O)[O-])NC1=O)C(=O)N[C@@H](CO)C(=O)N[C@@H](CC(=O)[O-])C(=O)N[C@@H]([C@@H](C)O)C(=O)N[C@@H](CC(C)C)C(=O)N2] |
| 69 | TSL6- SDCPWQSKTC | 17.69 | 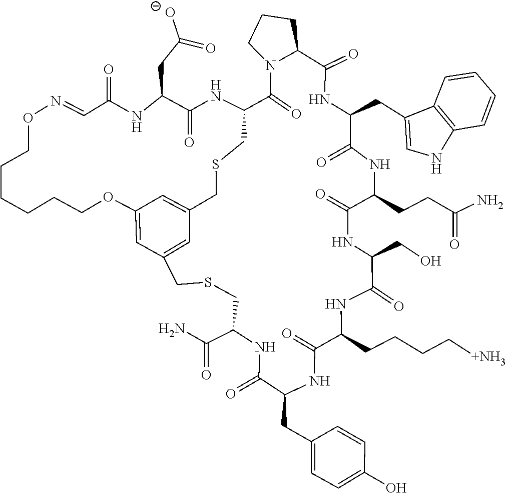 |
| 70 | Disulfide- RCS{Nle}DICRS LDVCWQ | 19.66 | 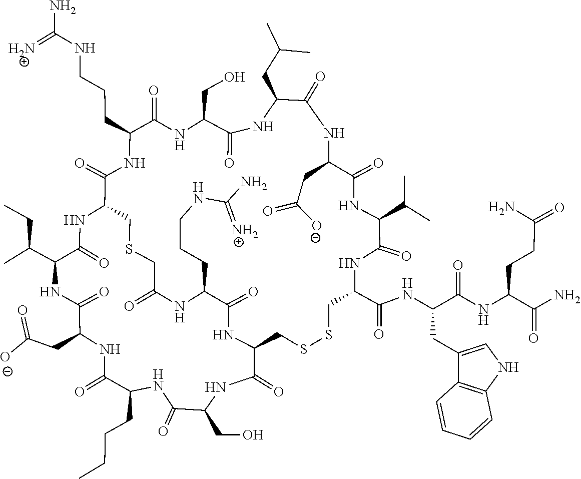 [SMILES: CCCC[C@@H]1NC(=O)[C@H](CO)NC(=O)[C@@H]2CSSC[C@@H](C(=O)N[C@@H](Cc3c[nH]c4ccccc34)C(=O)N[C@@H](CCC(N)=O)C(N)=O)NC(=O)[C@H](C(C)C)NC(=O)[C@@H](CC(=O)[O-])NC(=O)[C@H](CC(C)C)NC(=O)[C@H](CO)NC(=O)[C@H](CCCNC(N)=[NH2+])NC(=O)[C@H](CSCC(=O)N[C@@H](CCCNC(N)=[NH2+])C(=O)N2)NC(=O)[C@H]([C@@H](C)CC)NC(=O)[C@H](CC(=O)[O-])NC1=O] |
| 71 | TSL6- SECYPDTYHC | 20.18 | 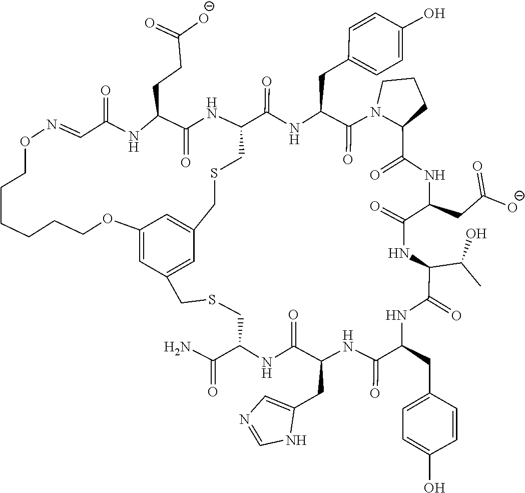 [SMILES: C[C@@H](O)[C@@H]1NC(=O)[C@H](CC(=O)[O-])NC(=O)[C@@H]2CCCN2C(=O)[C@H](Cc2ccc(O)cc2)NC(=O)[C@@H]2CSCc3cc(cc(c3)OCCCCCCO/N=C/C(=O)N[C@@H](CCC(=O)[O-])C(=O)N2)CSC[C@@H](C(N)=O)NC(=O)[C@H](Cc2cnc[nH]2)NC(=O)[C@H](Cc2ccc(O)cc2)NC1=O] |
| 72 | TSL6- SMCPLYIVHC | 29.38 | 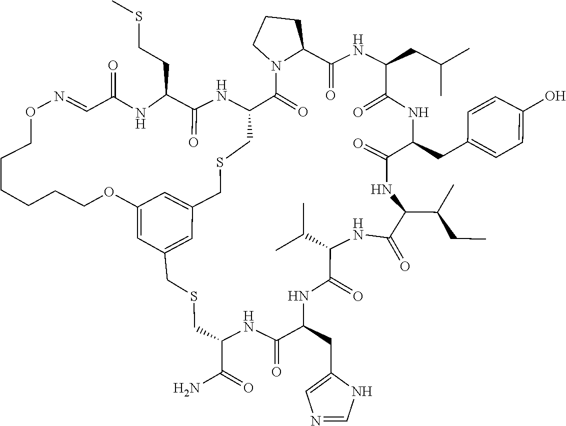 [SMILES: CC[C@H](C)[C@@H]1NC(=O)[C@H](Cc2ccc(O)cc2)NC(=O)[C@H](CC(C)C)NC(=O)[C@@H]2CCCN2C(=O)[C@@H]2CSCc3cc(cc(c3)OCCCCCCO/N=C/C(=O)N[C@@H](CCSC)C(=O)N2)CSC[C@@H](C(N)=O)NC(=O)[C@H](Cc2cnc[nH]2)NC(=O)[C@H](C(C)C)NC1=O] |
| 73 | TSL6- SLCYPDTYHC | 30.62 | 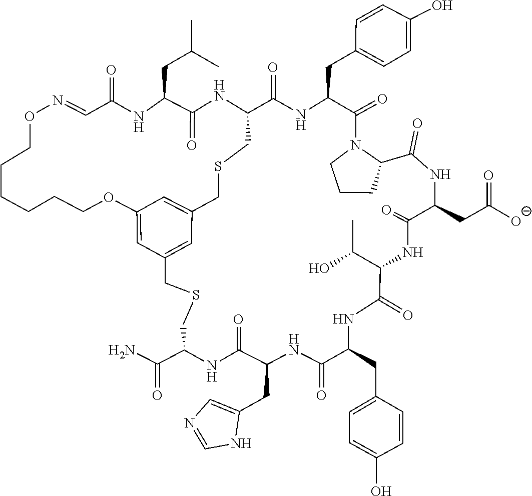 [SMILES: CC(C)C[C@@H]1NC(=O)/C=N/OCCCCCCOc2cc3cc(c2)CSC[C@H](NC1=O)C(=O)N[C@@H](Cc1ccc(O)cc1)C(=O)N1CCC[C@H]1C(=O)N[C@@H](CC(=O)[O-])C(=O)N[C@@H]([C@@H](C)O)C(=O)N[C@@H](Cc1ccc(O)cc1)C(=O)N[C@@H](Cc1cnc[nH]1)C(=O)N[C@H](C(N)=O)CSC3] |
| 74 | TSL6- SACPLDVPSC | 31 | 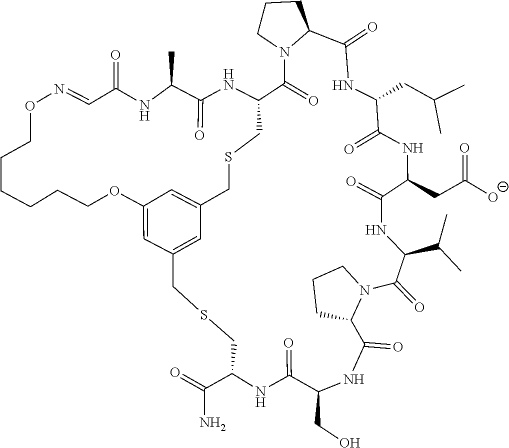 [SMILES: CC(C)C[C@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 75 | TSL6- SACPMDVWGEC | 31.32 | 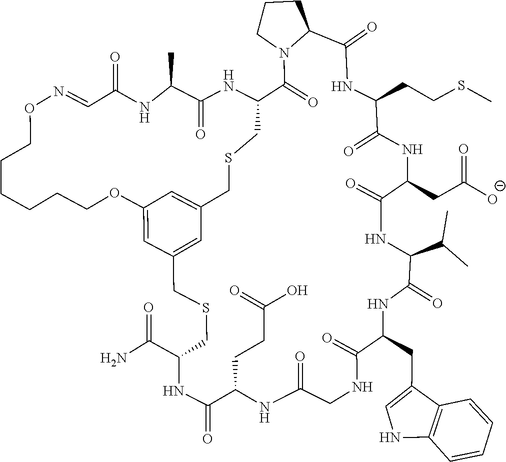 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)O)NC(=O)CNC(=O)[C@H](Cc2c[nH]c3ccccc23)NC(=O)[C@H](C(C)C)NC(=O)[C@H](CC(=O)[O-])NC1=O] |
| 76 | Disulfide- RCS{Nle}DICRS DTLCWQ | 34.3 | 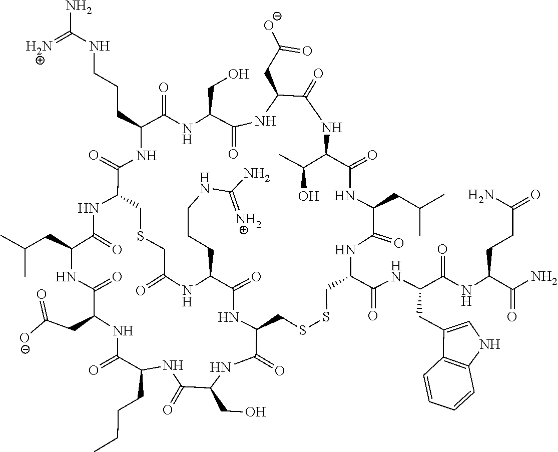 [SMILES: CCCC[C@@H]1NC(=O)[C@H](CO)NC(=O)[C@@H]2CSSC[C@@H](C(=O)N[C@@H](Cc3c[nH]c4ccccc34)C(=O)N[C@@H](CCC(N)=O)C(N)=O)NC(=O)[C@H](CC(C)C)NC(=O)[C@@H]([C@H](C)O)NC(=O)[C@H](CC(=O)[O-])NC(=O)[C@H](CO)NC(=O)[C@H](CCCNC(N)=[NH2+])NC(=O)[C@H](CSCC(=O)N[C@@H](CCCNC(N)=[NH2+])C(=O)N2)NC(=O)[C@H](CC(C)C)NC(=O)[C@H](CC(=O)[O-])NC1=O] |
| 77 | TSL6- SKCPADVPSC | 41.13 | 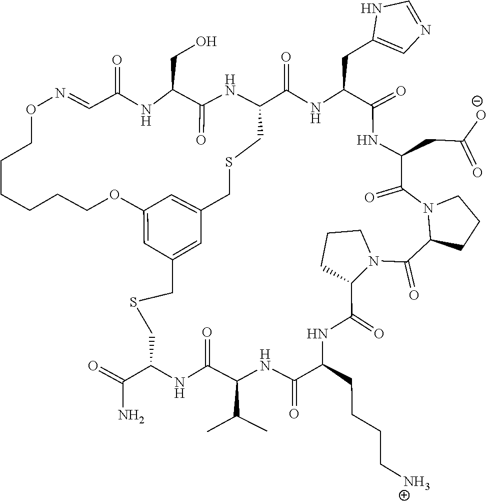 [SMILES: CC(C)[C@@H]1NC(=O)[C@H](CCCC[NH3+])NC(=O)[C@@H]2CCCN2C(=O)[C@@H]2CCCN2C(=O)[C@H](CC(=O)[O-])NC(=O)[C@H](Cc2cnc[nH]2)NC(=O)[C@@H]2CSCc3cc(cc(c3)OCCCCCCO/N=C/C(=O)N[C@@H](CO)C(=O)N2)CSC[C@@H](C(N)=O)NC1=O] |
| 78 | TSL6- SQCPADIPTC | 43.71 | 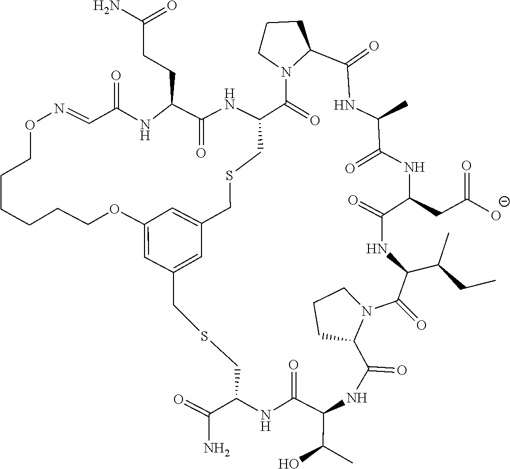 [SMILES: CC[C@H](C)[C@@H]1NC(=O)[C@H](CC(=O)[O-])NC(=O)[C@H](C)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 79 | TSL6- SDCYPDTFHC | 53.46 | 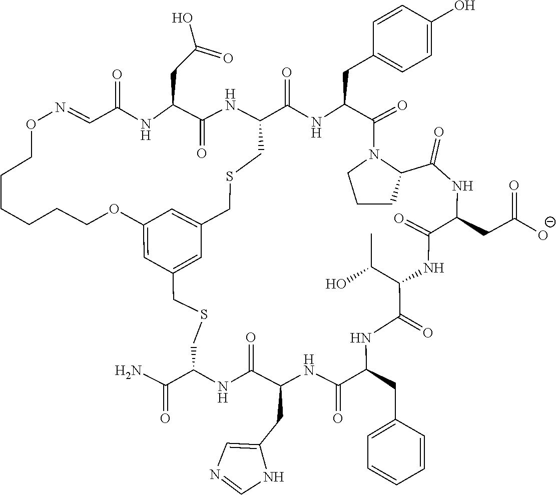 [SMILES: C[C@@H](O)[C@@H]1NC(=O)[C@H](CC(=O)[O-])NC(=O)[C@@H]2CCCN2C(=O)[C@H](Cc2ccc(O)cc2)NC(=O)[C@@H]2CSCc3cc(cc(c3)OCCCCCCO/N=C/C(=O)N[C@@H](CC(=O)O)C(=O)N2)CSC[C@@H](C(N)=O)NC(=O)[C@H](Cc2cnc[nH]2)NC(=O)[C@H](Cc2ccccc2)NC1=O] |
| 80 | TSLC7- GQCP{Hse(OMe)} D{tBuAla}PQEC | 55.9 | 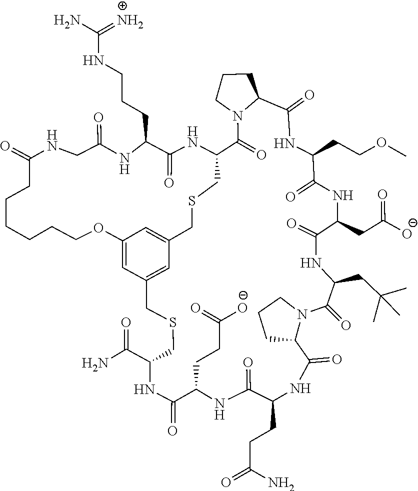 [SMILES: COCC[C@@H]1NC(=O)[C@@H]2CCCN2C(=O)[C@@H]2CSCc3cc(cc(c3)OCCCCCCC(=O)NCC(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@@H]2CCCN2C(=O)[C@H](CC(C)(C)C)NC(=O)[C@H](CC(=O)[O-])NC1=O] |
| 81 | TSL6- SKCPLAVPSC | 55.92 | 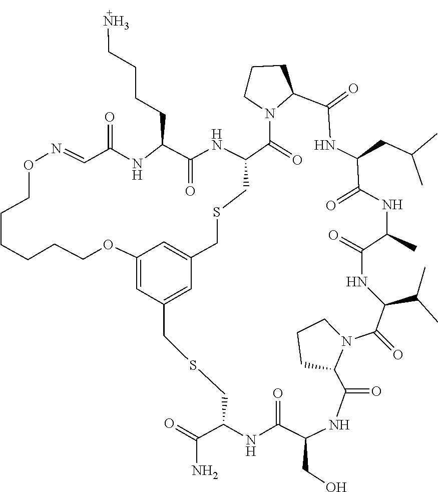 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCCN)C(=O)N2)CSC[C@@H](C(N)=O)NC(=O)[C@H](CO)NC(=O)[C@@H]2CCCN2C(=O)[C@H](C(C)C)NC(=O)[C@H](C)NC1=O] |
| 82 | TSL6- SGCMDDFAFC | 59.12 | 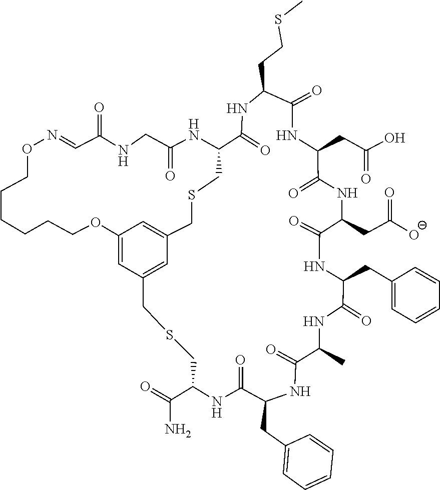 [SMILES: CSCC[C@@H]1NC(=O)[C@@H]2CSCc3cc(cc(c3)OCCCCCCO/N=C/C(=O)NCC(=O)N2)CSC[C@@H](C(N)=O)NC(=O)[C@H](Cc2ccccc2)NC(=O)[C@H](C)NC(=O)[C@H](Cc2ccccc2)NC(=O)[C@H](CC(=O)[O-])NC(=O)[C@H](CC(=O)O)NC1=O] |
| 83 | TSL6- SNCHWYGRIC | 63.83 | 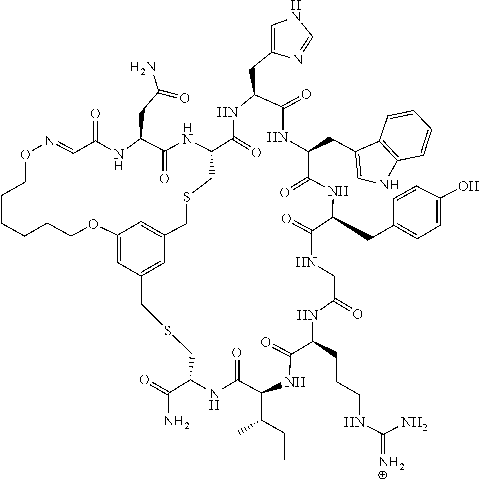 [SMILES: CC[C@H](C)[C@@H]1NC(=O)[C@H](CCCNC(N)=[NH2+])NC(=O)CNC(=O)[C@H](Cc2ccc(O)cc2)NC(=O)[C@H](Cc2c[nH]c3ccccc23)NC(=O)[C@H](Cc2c[nH]cn2)NC(=O)[C@@H]2CSCc3cc(cc(c3)OCCCCCCO/N=C/C(=O)N[C@@H](CC(N)=O)C(=O)N2)CSC[C@@H](C(N)=O)NC1=O] |
| 84 | TSL6- SDCFPDTYHC | 65.7 | 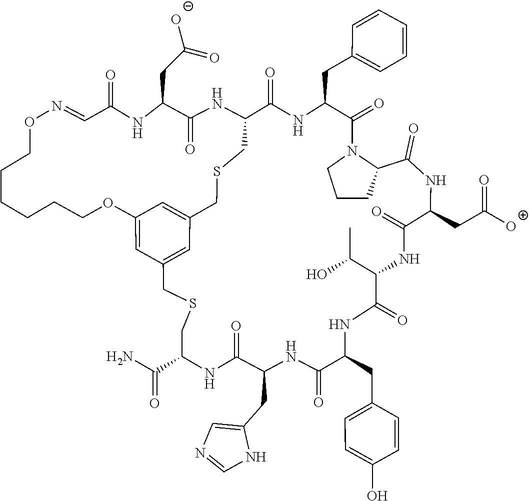 [SMILES: C[C@@H](O)[C@@H]1NC(=O)[C@H](CC(=O)[OH2+])NC(=O)[C@@H]2CCCN2C(=O)[C@H](Cc2ccccc2)NC(=O)[C@@H]2CSCc3cc(cc(c3)OCCCCCCO/N=C/C(=O)N[C@@H](CC(=O)[O-])C(=O)N2)CSC[C@@H](C(N)=O)NC(=O)[C@H](Cc2cnc[nH]2)NC(=O)[C@H](Cc2ccc(O)cc2)NC1=O] |
| 85 | TSL6- SACMLDVISC | 66.63 | 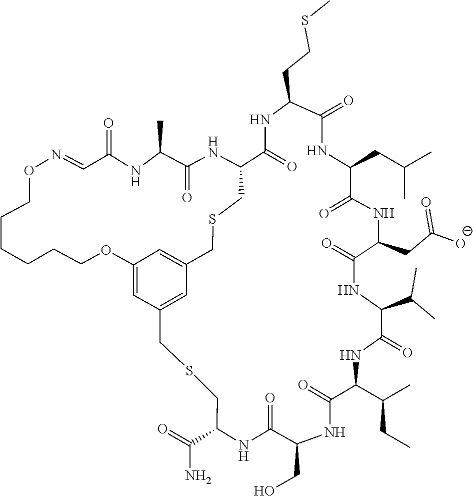 [SMILES: CC[C@H](C)[C@@H]1NC(=O)[C@H](C(C)C)NC(=O)[C@H](CC(=O)[O-])NC(=O)[C@H](CC(C)C)NC(=O)[C@H](CCSC)NC(=O)[C@@H]2CSCc3cc(cc(c3)OCCCCCCO/N=C/C(=O)N[C@@H](C)C(=O)N2)CSC[C@@H](C(N)=O)NC(=O)[C@H](CO)NC1=O] |
| 86 | TSL6- SACSDTGLQC | 70.71 | 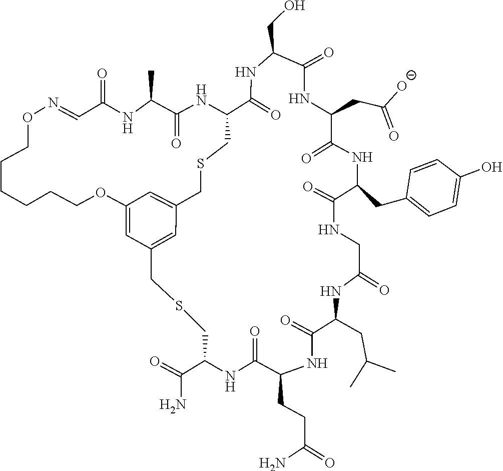 [SMILES: CC(C)C[C@@H]1NC(=O)CNC(=O)[C@H](Cc2ccc(O)cc2)NC(=O)[C@H](CC(=O)[O-])NC(=O)[C@H](CO)NC(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(N)=O)NC1=O] |
| 87 | Disulfide- RCSDTLCR SLDVCWQ | 71.31 | 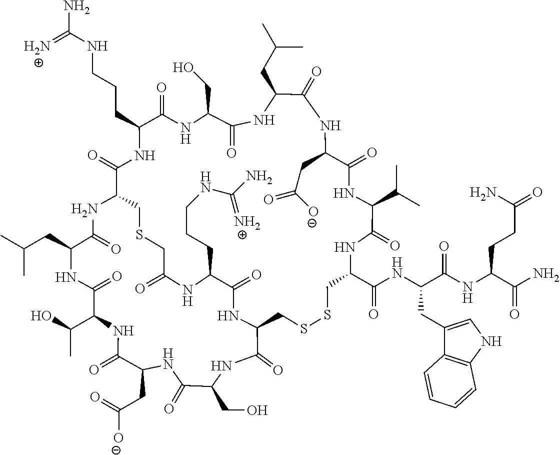 [SMILES: CC(C)C[C@@H]1NC(=O)[C@H](CO)NC(=O)[C@H](CCCNC(N)=[NH2+])NC(=O)[C@@H]2CSCC(=O)N[C@@H](CCCNC(N)=[NH2+])C(=O)N[C@@H](CSSC[C@@H](C(=O)N[C@@H](Cc3c[nH]c4ccccc34)C(=O)N[C@@H](CCC(N)=O)C(N)=O)NC(=O)[C@H](C(C)C)NC(=O)[C@@H](CC(=O)[O-])NC1=O)C(=O)N[C@@H](CO)C(=O)N[C@@H](CC(=O)[O-])C(=O)N[C@@H]([C@@H](C)O)C(=O)N[C@@H](CC(C)C)C(=O)N2] |
| 88 | TSL6- SDCYPDTYHC | 72.51 | 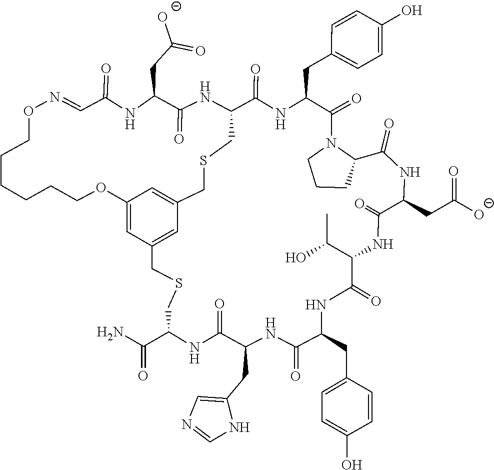 [SMILES: C[C@@H](O)[C@@H]1NC(=O)[C@H](CC(=O)[O-])NC(=O)[C@@H]2CCCN2C(=O)[C@H](Cc2ccc(O)cc2)NC(=O)[C@@H]2CSCc3cc(cc(c3)OCCCCCCO/N=C/C(=O)N[C@@H](CC(=O)[O-])C(=O)N2)CSC[C@@H](C(N)=O)NC(=O)[C@H](Cc2cnc[nH]2)NC(=O)[C@H](Cc2ccc(O)cc2)NC1=O] |
| 89 | TSL6- SNCYPDTYHC | 79.31 | 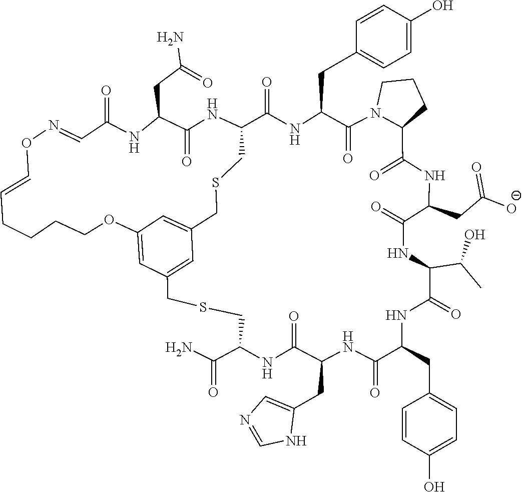 [SMILES: C[C@@H](O)[C@@H]1NC(=O)[C@H](CC(=O)[O-])NC(=O)[C@@H]2CCCN2C(=O)[C@H](Cc2ccc(O)cc2)NC(=O)[C@@H]2CSCc3cc(cc(c3)OCCCC/C=C/O/N=C/C(=O)N[C@@H](CC(N)=O)C(=O)N2)CSC[C@@H](C(N)=O)NC(=O)[C@H](Cc2cnc[nH]2)NC(=O)[C@H](Cc2ccc(O)cc2)NC1=O] |
| 90 | TSL6- SACRSDTLC | 79.77 | 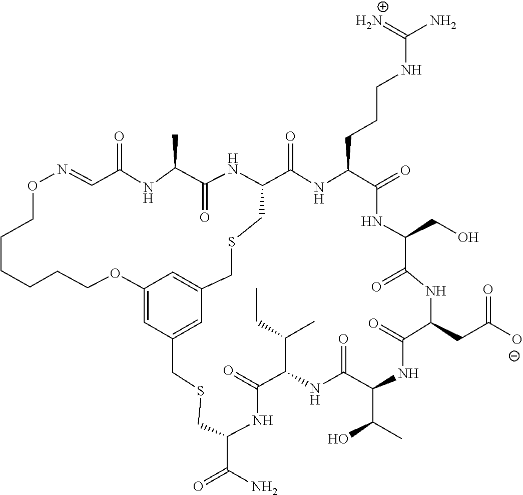 [SMILES: CC[C@H](C)[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]2CSCc3cc(cc(c3)OCCCCCCO/N=C/C(=O)N[C@@H](C)C(=O)N2)CSC[C@@H](C(N)=O)NC1=O] |
| 91 | TSL6- SICYPDTYHC | 93.22 | 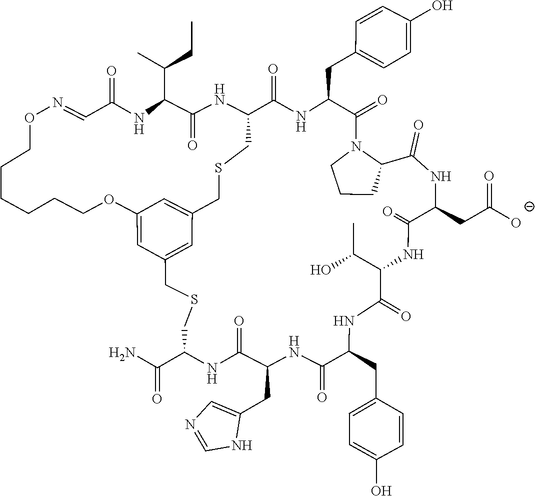 [SMILES: CC[C@H](C)[C@@H]1NC(=O)/C=N/OCCCCCCOc2cc3cc(c2)CSC[C@H](NC1=O)C(=O)N[C@@H](Cc1ccc(O)cc1)C(=O)N1CCC[C@H]1C(=O)N[C@@H](CC(=O)[O-])C(=O)N[C@@H]([C@@H](C)O)C(=O)N[C@@H](Cc1ccc(O)cc1)C(=O)N[C@@H](Cc1cnc[nH]1)C(=O)N[C@H](C(N)=O)CSC3] |
| 92 | TSL6- SRCLDIMYDC | 100.08 | 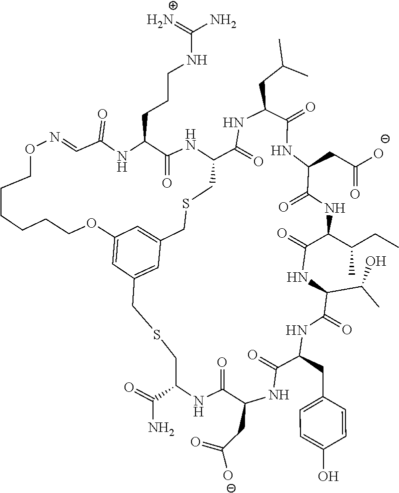 [SMILES: CC[C@H](C)[C@@H]1NC(=O)[C@H](CC(=O)[O-])NC(=O)[C@H](CC(C)C)NC(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CC(=O)[O-])NC(=O)[C@H](Cc2ccc(O)cc2)NC(=O)[C@H]([C@@H](C)O)NC1=O] |
| 93 | TSL6- SACYPDTYHC | 111.5 | 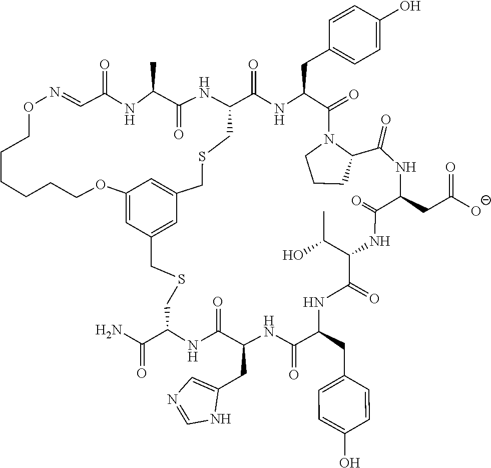 [SMILES: C[C@@H]1NC(=O)/C=N/OCCCCCCOc2cc3cc(c2)CSC[C@H](NC1=O)C(=O)N[C@@H](Cc1ccc(O)cc1)C(=O)N1CCC[C@H]1C(=O)N[C@@H](CC(=O)[O-])C(=O)N[C@@H]([C@@H](C)O)C(=O)N[C@@H](Cc1ccc(O)cc1)C(=O)N[C@@H](Cc1cnc[nH]1)C(=O)N[C@H](C(N)=O)CSC3] |
| 94 | TSL1- SACRSDTLC | 111.5 | 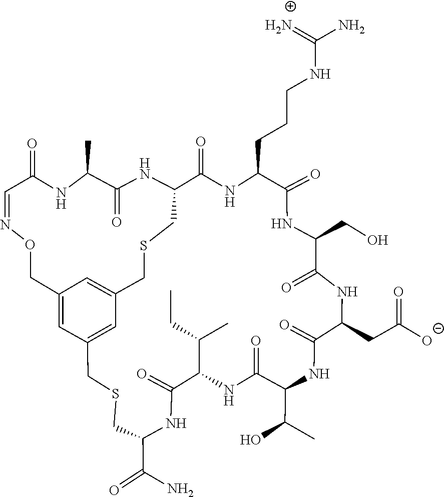 [SMILES: CC[C@H](C)[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]2CSCc3cc(cc(c3)CSC[C@@H](C(N)=O)NC1=O)CO/N=C\C(=O)N[C@@H](C)C(=O)N2] |
| 95 | TSL6- SDCWPDTYHC | 113.8 | 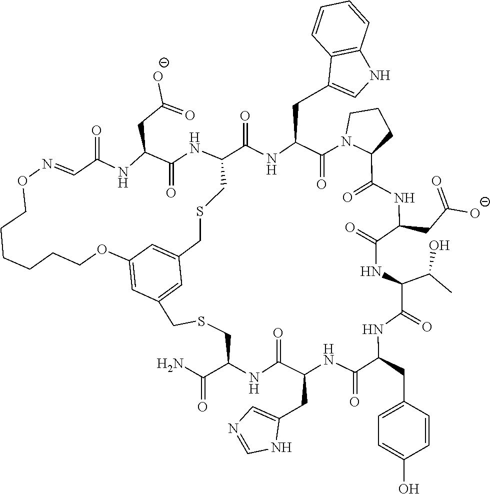 [SMILES: C[C@@H](O)[C@@H]1NC(=O)[C@H](CC(=O)[O-])NC(=O)[C@@H]2CCCN2C(=O)[C@H](Cc2c[nH]c3ccccc23)NC(=O)[C@@H]2CSCc3cc(cc(c3)OCCCCCCO/N=C/C(=O)N[C@@H](CC(=O)[O-])C(=O)N2)CSC[C@H](C(N)=O)NC(=O)[C@H](Cc2cnc[nH]2)NC(=O)[C@H](Cc2ccc(O)cc2)NC1=O] |
| 96 | TSL6- SDCYPDFYHC | 124.5 | 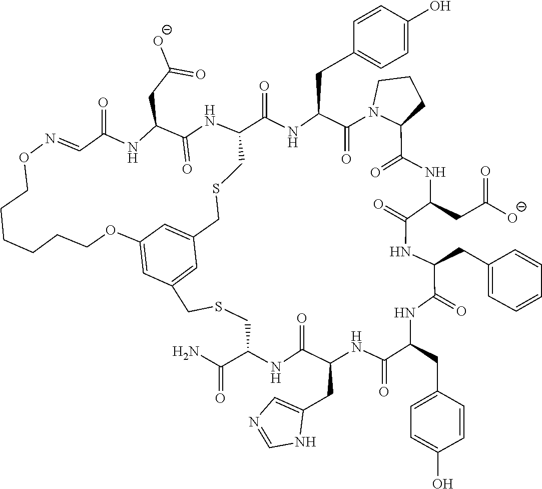 [SMILES: NC(=O)[C@@H]1CSCc2cc3cc(c2)OCCCCCCO/N=C/C(=O)N[C@@H](CC(=O)[O-])C(=O)N[C@@H](CSC3)C(=O)N[C@@H](Cc2ccc(O)cc2)C(=O)N2CCC[C@H]2C(=O)N[C@@H](CC(=O)[O-])C(=O)N[C@@H](Cc2ccccc2)C(=O)N[C@@H](Cc2ccc(O)cc2)C(=O)N[C@@H](Cc2cnc[nH]2)C(=O)N1] |
| 97 | TSL6- SFCLGYEASC | 127.2 | 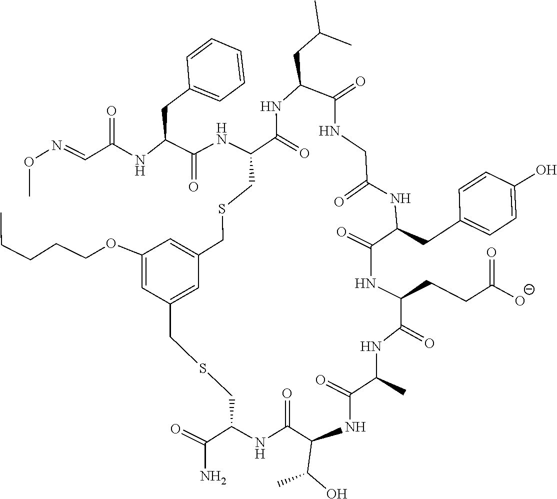 [SMILES: CCCCCOc1cc2cc(c1)CSC[C@H](NC(=O)[C@H](Cc1ccccc1)NC(=O)/C=N/OC)C(=O)N[C@@H](CC(C)C)C(=O)NCC(=O)N[C@@H](Cc1ccc(O)cc1)C(=O)N[C@@H](CCC(=O)[O-])C(=O)N[C@@H](C)C(=O)N[C@@H]([C@@H](C)O)C(=O)N[C@H](C(N)=O)CSC2] |
| 98 | TSL6- SMCPLYKVHC | 151.5 | 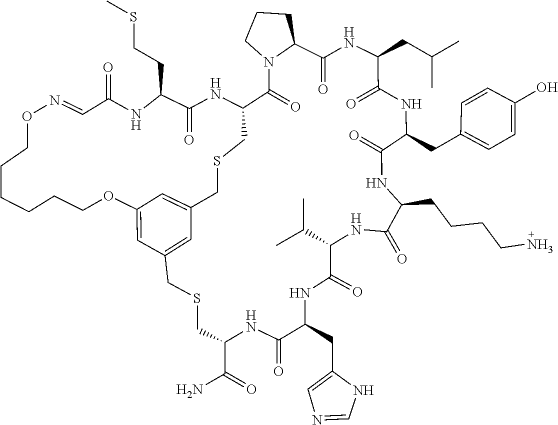 [SMILES: CSCC[C@@H]1NC(=O)/C=N/OCCCCCCOc2cc3cc(c2)CSC[C@H](NC1=O)C(=O)N1CCC[C@H]1C(=O)N[C@@H](CC(C)C)C(=O)N[C@@H](Cc1ccc(O)cc1)C(=O)N[C@@H](CCCCN)C(=O)N[C@@H](C(C)C)C(=O)N[C@@H](Cc1cnc[nH]1)C(=O)N[C@H](C(N)=O)CSC3] |
| 99 | TSL6- SQCP{Ox-Met} AIPTC | 152.3 | 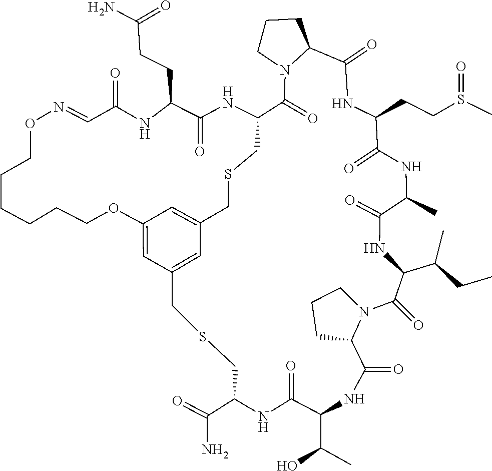 [SMILES: CC[C@H](C)[C@@H]1NC(=O)[C@H](C)NC(=O)[C@H](CCS(C)=O)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 100 | TSL6- SFCGATMKKC | 162.1 | 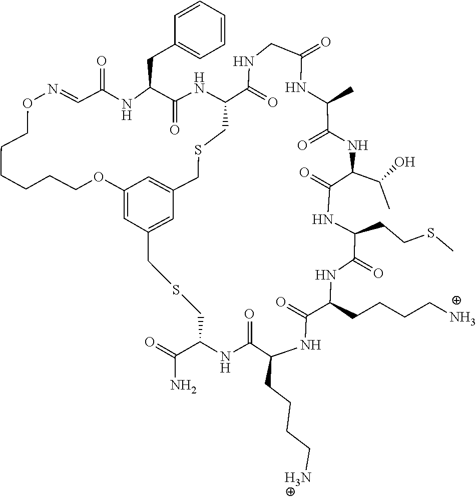 [SMILES: CSCC[C@@H]1NC(=O)[C@H]([C@@H](C)O)NC(=O)[C@H](C)NC(=O)CNC(=O)[C@@H]2CSCc3cc(cc(c3)OCCCCCCO/N=C/C(=O)N[C@@H](Cc3ccccc3)C(=O)N2)CSC[C@@H](C(N)=O)NC(=O)[C@H](CCCC[NH3+])NC(=O)[C@H](CCCC[NH3+])NC1=O] |
| 101 | TSL6- SMCYPDTYHC | 167.4 | 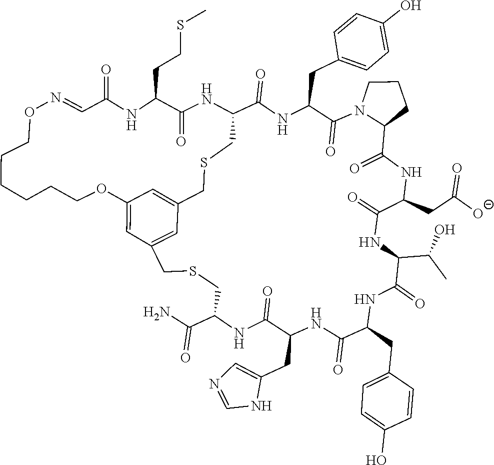 [SMILES: CSCC[C@@H]1NC(=O)/C=N/OCCCCCCOc2cc3cc(c2)CSC[C@H](NC1=O)C(=O)N[C@@H](Cc1ccc(O)cc1)C(=O)N1CCC[C@H]1C(=O)N[C@@H](CC(=O)[O-])C(=O)N[C@@H]([C@@H](C)O)C(=O)N[C@@H](Cc1ccc(O)cc1)C(=O)N[C@@H](Cc1cnc[nH]1)C(=O)N[C@H](C(N)=O)CSC3] |
| 102 | TSL6- SFCQMFEKAC | 177.5 | 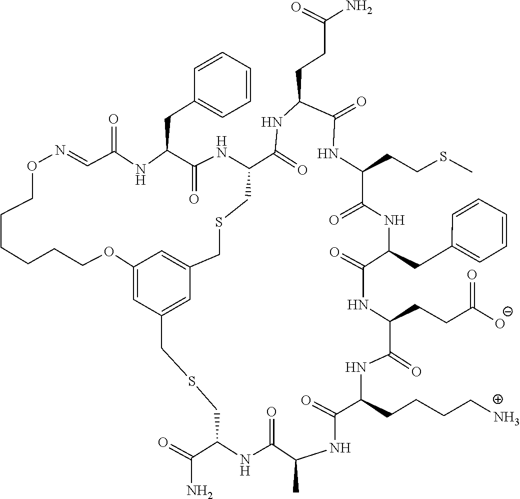 [SMILES: CSCC[C@@H]1NC(=O)[C@H](CCC(N)=O)NC(=O)[C@@H]2CSCc3cc(cc(c3)OCCCCCCO/N=C/C(=O)N[C@@H](Cc3ccccc3)C(=O)N2)CSC[C@@H](C(N)=O)NC(=O)[C@H](C)NC(=O)[C@H](CCCC[NH3+])NC(=O)[C@H](CCC(=O)[O-])NC(=O)[C@H](Cc2ccccc2)NC1=O] |
| 103 | Disulfide- RCS{Nle}DICRS NleDICWQ | 263.4 | 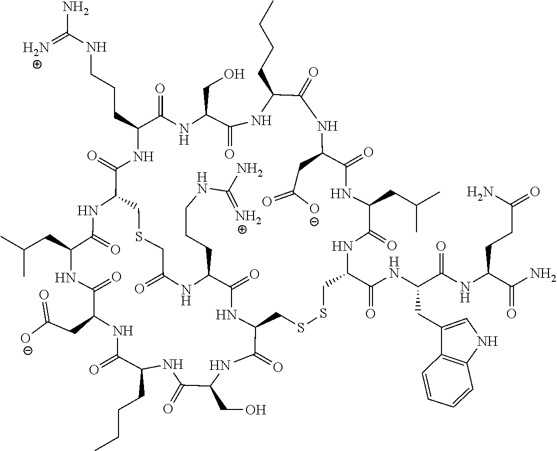 [SMILES: CCCC[C@@H]1NC(=O)[C@H](CO)NC(=O)[C@@H]2CSSC[C@@H](C(=O)N[C@@H](Cc3c[nH]c4ccccc34)C(=O)N[C@@H](CCC(N)=O)C(N)=O)NC(=O)[C@H](CC(C)C)NC(=O)[C@@H](CC(=O)[O-])NC(=O)[C@H](CCCC)NC(=O)[C@H](CO)NC(=O)[C@H](CCCNC(N)=[NH2+])NC(=O)[C@H](CSCC(=O)N[C@@H](CCCNC(N)=[NH2+])C(=O)N2)NC(=O)[C@H](CC(C)C)NC(=O)[C@H](CC(=O)[O-])NC1=O] |
| 104 | TSL6- STCFLDVKNC | 310.62 | 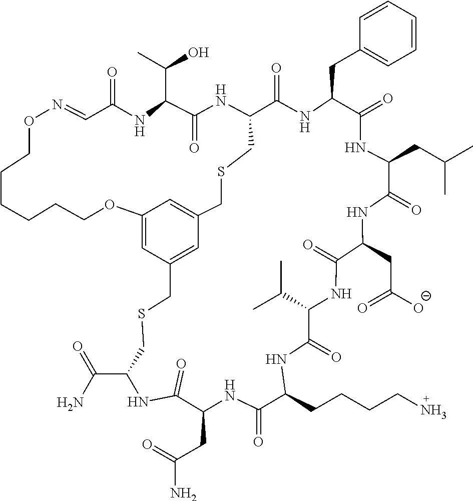 [SMILES: CC(C)C[C@@H]1NC(=O)[C@H](Cc2ccccc2)NC(=O)[C@@H]2CSCc3cc(cc(c3)OCCCCCCO/N=C/C(=O)N[C@@H]([C@@H](C)O)C(=O)N2)CSC[C@@H](C(N)=O)NC(=O)[C@H](CC(N)=O)NC(=O)[C@H](CCCC[NH3+])NC(=O)[C@H](C(C)C)NC(=O)[C@H](CC(=O)[O-])NC1=O] |
| 105 | TSL6- SECPWPLPQC | 341.48 | 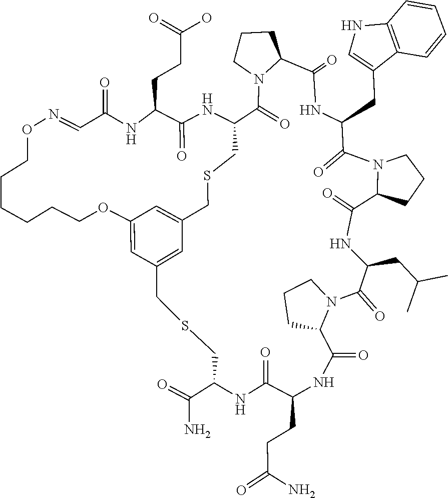 |
| 106 | TSL6- STCRMDFQDC | 499.3 | 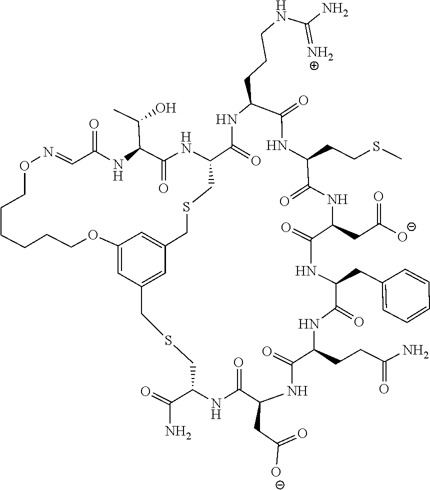 [SMILES: CSCC[C@@H]1NC(=O)[C@H](CCCNC(N)=[NH2+])NC(=O)[C@@H]2CSCc3cc(cc(c3)OCCCCCCO/N=C/C(=O)N[C@@H]([C@H](C)O)C(=O)N2)CSC[C@@H](C(N)=O)NC(=O)[C@H](CC(=O)[O-])NC(=O)[C@H](CCC(N)=O)NC(=O)[C@H](Cc2ccccc2)NC(=O)[C@H](CC(=O)[O-])NC1=O] |
| 107 | TSL6- SHCKLDIWVC | >500 | 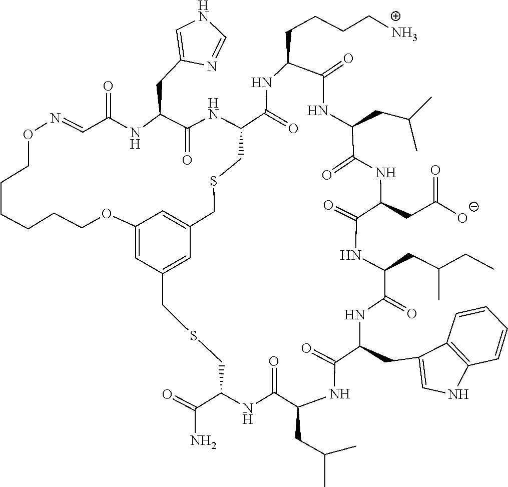 [SMILES: CCC(C)C[C@@H]1NC(=O)[C@H](CC(=O)[O-])NC(=O)[C@H](CC(C)C)NC(=O)[C@H](CCCC[NH3+])NC(=O)[C@@H]2CSCc3cc(cc(c3)OCCCCCCO/N=C/C(=O)N[C@@H](Cc3c[nH]cn3)C(=O)N2)CSC[C@@H](C(N)=O)NC(=O)[C@H](CC(C)C)NC(=O)[C@H](Cc2c[nH]c3ccccc23)NC1=O] |
| 108 | TSL6- SKCPLATPYEC | >500 | 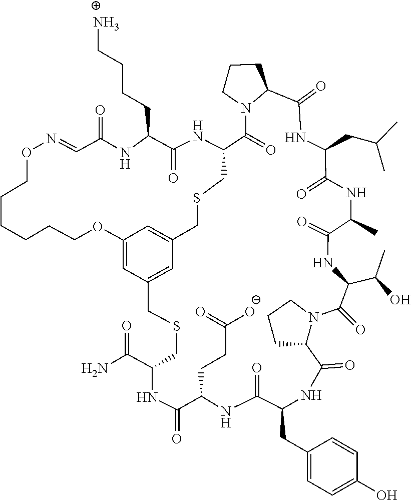 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](C)NC1=O] |
| 109 | TSL- 6SKCPADTPYEC | >500 | 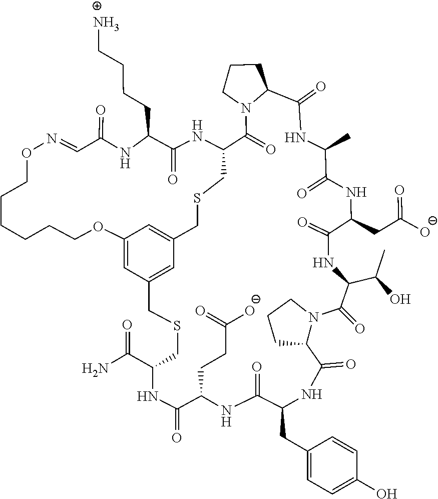 [SMILES: C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 110 | TSL6- SQCPMAIPTC | >500 | 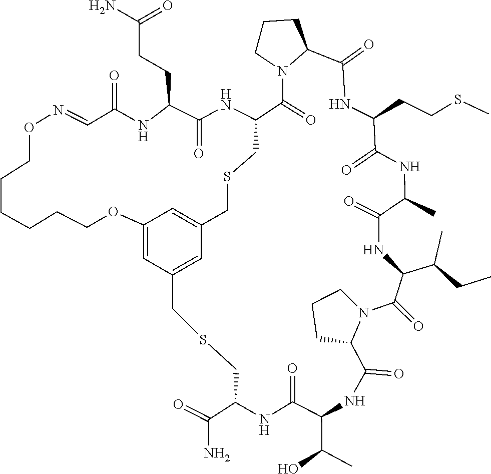 [SMILES: CC[C@H](C)[C@@H]1NC(=O)[C@H](C)NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 111 | TSL6- SQCHWYGRSC | >500 | 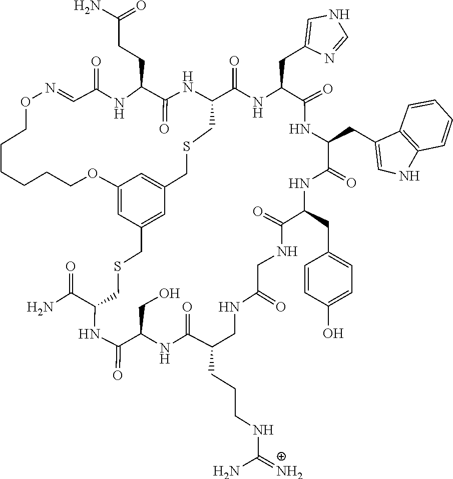 [SMILES: NC(=[NH2+])NCCC[C@H]1CNC(=O)CNC(=O)[C@H](Cc2ccc(O)cc2)NC(=O)[C@H](Cc2c[nH]c3ccccc23)NC(=O)[C@H](Cc2c[nH]cn2)NC(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@@H](CO)NC1=O] |

### Phage Display and Panning

Phage display is the preferred method for discovering high affinity peptide binders to protein targets. The protocol for panning described herein is the same for linear, cyclic, and bicyclic compounds, except that the phage library is chemically modified when panning for bicyclic molecules.

M13 Phage Libraries were used for displaying randomized peptide sequences fused to the pill protein. Libraries were chemically modified before panning. First, the phage library was exposed to NaIO₄ (60 uM in PBS for 10 min, then quenched with 0.5 mM methionine for 20 min) to convert the N-terminal serine to a reactive aldehyde group. TSL linker was added at 1 mM and pH 3.5 for an hour to react the aminooxy functional group to the N-terminal aldehyde. The reacted phage library was then exposed to TCEP and the pH of the library was increased to pH 10 to force bicyclization. Efficiency of the reaction was monitored using aminooxybiotin and a biotin thiol reagent, followed by capture using streptavidin beads.

Panning was performed in three rounds with the target bound to a 96-well plate in Round 1 and beads in Rounds 2 and 3. A solution of 100 μg/mL of target protein in buffer (eg. 20 μL of a 0.5 μg/μL protein solution plus 80 μL of PBS) was added to 100 μL protein/well, making 4 replicate wells in total. Plates were sealed and incubated at 4° C. overnight.

Two E. coli overnight cultures were prepared as follows: a. TG1 prAl4 for phage amplification and b. ER2738 for phage titering by plaque assay. Using a sterile plastic loop, a single colony was picked from the appropriate master plate into 7 mL LB in a polystyrene 15 mL snap cap tube. Both cultures were incubated in a tube rack in a floor shaker at 37° C. with shaking and allowed to grow overnight for about 12-13 hours.

Target protein/plates were blocked with BSA by removing supernatant and adding 100 μL of 2% BSA/PBS blocking solution to each well. Each plate was covered and incubated at room temperature for 1 hour. The phage library was diluted to 10¹² PFU/ml in blocking solution and 100 μL of the diluted library was added to each well and covered and incubated at room temperature for 1.5 hours. Unbound phage was washed using 0.1% Tween-20 in PBS wash solution and the phage libraries were eluted from the target/plate with 0.2M glycine-HCL 0.1% BSA followed by neutralizing with 30 μL of 1 M Tris-HCl pH 9.1. Three aliquots were made for phage tittering, PCR, and phage amplification.

### Round 2 Panning:

Following PCR amplification followed by PEG precipitation, target proteins were bound to beads for Round 2 and 3 of panning, with beads only serving as the negative control. Beads were selected based on the type of protein target tag. Solutions of 100 μg/mL of 10 μg target protein in buffer were added to the beads, and buffer alone was added to the negative control. All protein-bead and bead only solutions were incubated overnight at 4° C. on an end-to-end rotator in a cold room. Phage libraries were generally depleted of non-specific bead binders prior to panning in Round 2 and Round 3. 100 uL of beads were washed in PBS and mixed with beads containing no protein to remove non-specific bead binders. After removing supernatant, 200-300 μL of the Round 2 input library was added to the beads and the library/breads solution was incubated overnight at 4° C. in a cold room. Two E. coli overnight cultures were set up: a. TG1 prAl4 for phage amplification and b. ER2738 for phage titering by plaque assay. Bead/protein solutions were added to a 96 well plate and the library was diluted into 2% BSA in buffer. Plates were washed in protein/beads were transferred to individually labeled tubes, followed by acid and boiling elution (both Round 2 and Round 3), and generating 3 aliquots from each elution for titering in ER2738 plaque assays, amplification and PCR.

### Round 3 Panning:

Round 3 panning is the same as round 2 panning except for performing an additional wash, and resulting in acid and boiling elution tubes for amplification in 250 μl TG1 prAl4, 200 μL phage, titering in ER2738 by plaque assay, and PCR/sequencing. Panning ends with a final PEG/NaCL precipitation as in previous rounds.

Samples collected for PCR were amplified and analyzed by next generation sequencing using Illumina technology. Abundances of specific peptide sequences from the R2 and R3 panning were normalized to input controls and compared to normalized pools from beads or plate alone (no protein). Peptide sequences exhibiting significant enrichment in the target-specific pools were considered for further evaluation.

Positional scan: Significantly enriched peptides were selected to construct a focus library consisting of a positional scan. Each amino acid in the peptide (except for Cys residues and the N-terminal Ser) was replaced with every other amino acid (except Cys). A phage display library was constructed, modified with the TSL-6 linker, and panned for a single round (methods similar as above). NGS bioinformatics was used to look at the relative abundance of each peptide analog in the naïve library and in the enriched library. Amino acid substitutions that resulted in enrichment were predicted to have better binding affinity, and substitutions that caused depletion were considered to be deleterious.

### Example 1

### Competition Binding Assay

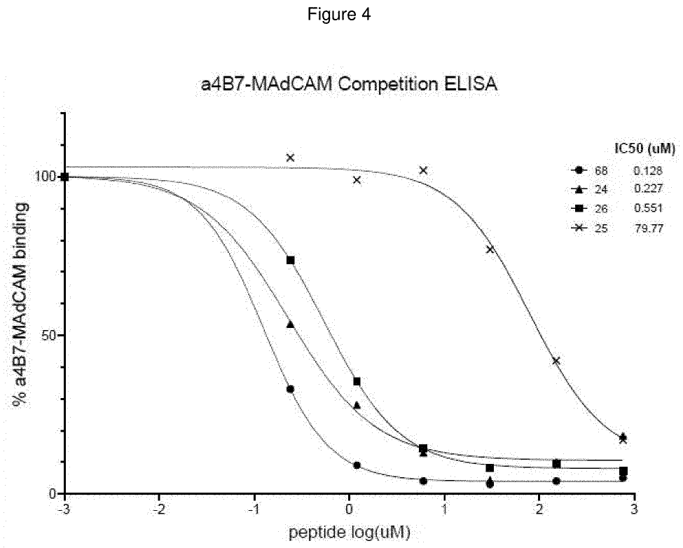

A competition ELISA was performed by coating a 96 well plate with 100 μL of 8 μg/mL rh-integrin α4β7/LPAM-1 for one hour at 25° C. while shaking. After washing with wash buffer, 200 μL block/assay buffer was added to each well comprising 50 μL of 1 μg/mL MAdCAM in assay buffer, 50 μL of peptide stock in 10 mM DMSO diluted to 1 mM assay buffer, and a DMSO control in assay buffer and incubated for one hour at 25° C. while shaking, followed by washing. 50 μL of anti-human IgG1 diluted at 1:2000 in assay buffer was added followed by TMB solution and incubation at 25° C. for 15-30 minutes. After the reaction was stopped, absorbance was measured at 450 nm. Results are shown in FIG. 4 and Table 3.

TABLE 3
IC50

| Sequence | IC50 (uM) | mIC50 (uM) |
|---|---|---|
| SEQ ID NO: 67 | 0.128 | |
| SEQ ID NO: 65 | 0.136 | |
| SEQ ID NO: 66 | 0.164 | |
| SEQ ID NO: 69 | 0.186 | |
| SEQ ID NO: 24 | 0.227 | 0.88 |
| SEQ ID NO: 131 | 0.551 | 1 |
| SEQ ID NO: 26 | 0.631 | 1.15 |
| SEQ ID NO: 23 | 1.325 | 2.27 |
| SEQ ID NO: 60 | 5.432 | |
| SEQ ID NO: 61 | 6.419 | |
| SEQ ID NO: 12 | 59.12 | 53.2 |
| SEQ ID NO: 11 | 66.63 | 37.77 |
| SEQ ID NO: 25 | 79.77 | |
| SEQ ID NO: 16 | 89.95 | 129.2 |
| SEQ ID NO: 70 | 111.5 | |
| SEQ ID NO: 8 | 151.4 | |

### Example 2

### Simulated Intestinal Fluid Assay

Studies were carried out in simulated intestinal fluid (SIF) to evaluate the intestinal stability of the peptide molecules of the peptides of the instant disclosure. A Fasted State Simulated Intestinal Fluid (FaSSIF) reagent was prepared according to methods known in the art (including addition of the enzyme pancreatin), to which stock peptide solutions were added, incubated, quenched, and analyzed using LCMS/MS. Two controls were used: chlorambucil known to degrade in SIF and warfarin known to be stable in SIF.

Percent remaining at each time point was calculated based on the peak area response ratio of test to compound to internal standard. Half-lives were calculated by fitting to a first-order exponential decay equation using GraphPad. The result are shown in Table 4.

TABLE 4
Simulated Intestinal Fluid

| | | Half Life | | |
|---|---|---|---|---|
| Sequence | Matrix | (min) | Transition | Comments |
| SEQ ID NO. 13 | FaSSIF | | | No peak |
| | | | | detected |
| SEQ ID NO: 22 | FaSSIF | 72.3 | 631.5/600.1 | |
| SEQ ID NO. 14 | FaSSIF | 79.4 | 600.6/157.1 | |
| SEQ ID NO. 16 | FaSSIF | 207 | 694.1/136.1 | |
| SEQ ID NO. 5 | FaSSIF | 23.6 | 589.6/143.1 | |
| SEQ ID NO. 8 | FaSSIF | 8.23 | 682.6/659.7 | |
| SEQ ID NO. 15 | FaSSIF | 64.3 | 672.1/70.0 | |
| SEQ ID NO. 19 | FaSSIF | >480 | 706.4/689.2 | |
| SEQ ID NO. 20 | FaSSIF | 81.5 | 669.6/651.7 | |
| SEQ ID NO. 21 | FaSSIF | 19.7 | 694.6/597.7 | |
| Chlorambucil | FaSSIF | 37.6 | 304.1/168.1 | Assay Control |
| Warfarin | FaSSIF | >480 | 309.0/163.0 | Assay Control |

### Example 3—Prophetic Examples

In addition, Table 5 describes additional peptide scaffold sequences of the present disclosure. These sequences were designed using a next generation sequencing protocol (NGS protocol), in which Fastq files generated through Paired-end Deep Sequencing will be parsed and aligned to obtain the abundance (raw read counts) of each individual sequence in the input, control, and test samples respectively (He, B. et al., (2018)). Differential Enrichment Analysis (DEA) will be applied to normalize the abundance first by using the Trimmed Mean of M-values (TMM) method and to fit the normalized abundance into a quasi-likelihood negative binomial generalized log-linear model to obtain fold change (FC) and its corresponding p-values of the test/Input and control/Input, normalized counts per million (CPM) for each sequence in the dataset. FC, p-values and CPM are further converted into probability scores (Confidence Scores (CS)) through a set of sigmoid functions. Confidence scores (CS) represent the estimated likelihood of a peptide to be a binder to the target. Sequences with the highest CS will be selected as top candidate peptide scaffolds.

Positional scans, where the amino acid at a specific position is replaced by the rest of 19 amino acids, will be applied to initial hits. Heat maps comparing the positional scanned peptides to the parent peptides will be generated for visualizations with various metrics.

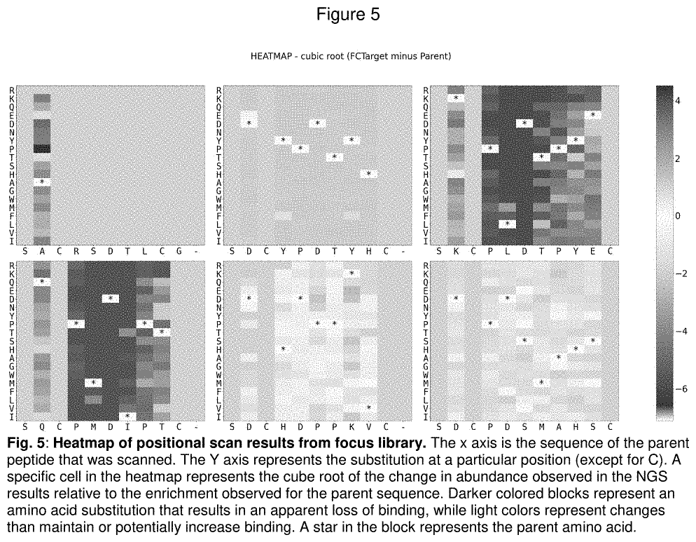

FIG. 5 describes heat maps of positional scans generated for the compounds of the present disclosure comprising peptide sequences of SEQ ID NOs. 16, 23, 131, 61, 62, and 68.

The peptide scaffold sequences of the present disclosure will be positionally scanned as described above and the biophysical characteristics of the cyclic or bicyclic compounds will be elucidated using a variety of experiments and assays.

TABLE 5
Future Peptide Sequences

| Sequence* | Linker | Target |
|---|---|---|
| SXCRSDTLC | TSL-1, TSL-3, TSL-6 | α4β7 |
| SXCYPDTYHC | TSL-6 | α4β7 |
| SXCPMDIAGEC | TSL-6 | α4β7 |
| SXCPLDVPSC | TSL-6 | α4β7 |
| SXCPLDTPYEC | TSL-6 | α4β7 |
| SXCPMDIPTC | TSL-6 | α4β7 |

*X represents replacement of the amino acid residue at that position, and each sequence will be scanned in its entirety.

Various biophysical properties of the compounds described herein were measured using the assays described herein, and other assays, including the following: Biacore™ Binding assay

Binding experiments will be carried out on the compounds described herein, by Surface Plasmon Resonance using a Biacore™ 3000 instrument (GE Healthcare). α4β7 biotinylated will be immobilized on a Sensor Chip SA (Cytivia). The compounds will be flowed across this chip to determine the binding affinity to α4β7 and/or MAdCAM. Binding measurements will be done with concentrations from 50 μM-100 μM in HBS-PMn buffer at 25° C. Diluted compounds will be injected over FC1 and FC2 at a flow rate of 20 μL/mL. The association phase of the injection will last 90 seconds, and the dissociation phase will last 180 seconds. If a compound remains bound after 180 seconds, additional time will be given to fully dissociate the compound (i.e., the signal had returned to baseline) before the next injection. No regeneration will be performed. Data analysis will be performed using the Biacore software. Curve fitting will be done using a 1:1 binding model, with the off-rates and on-rates fit separately.

### α4β7-MAdCAM Competition ELISA

rh integrin α4β7 (R&D Systems #5397-A3) at 400 ng/well will be added to a nickel coated plate (Pierce #15442), then incubated while shaking at room temperature for 1 hour. The solution will be removed and the each well of the plate was washed 3 times with 200 μL of wash buffer (50 mM Tris-HCl pH 7.6, 100 mM NaCl, 1 mM MnCl₂, 0.05% Tween-20). Assay buffer (50 mM Tris-HCl pH 7.6, 150 mM NaCl, 1 mM MnCl₂, 0.05% Tween-20, 0.5% BSA) will be added 200 μL/well and incubated at room temperature for 2 hours to block, then each well will be washed 3 times with wash buffer. A mixture of 25 μL rh MAdCAM-1 (R&D Systems #6056-MC) at 1 μg/mL and 25 μL of compound at 5-fold serial dilutions starting at 750 μM in assay buffer will be added to each well and incubated at room temperature for 1 hour, then each well will be washed 3 times with wash buffer. 100 μL of mouse anti-human IgG1-HRP (Invitrogen #A10648) diluted at 1:4000 in assay buffer will be added to the wells and incubated while shaking at room temperature for 45 minutes, then each well will be washed with was buffer 3 times. 100 μL of TMB will be added to each well and incubated at room temperature 15-30 minutes, then the reaction will be stopped with 100 μL of TMB stop solution (2N H₂SO₄). Absorbance will be read at 450 nm.

### Example 4

### α4β7-MAdCAM Cell Adhesion Assay

RPMI 8866 cells (Sigma #95041316) will be cultured in RPMI 1640 HEPES medium (Invitrogen #22400-089) supplemented with 10% Fetal Bovine Serum (Invitrogen #16140-071), 1 mM sodium pyruvate (Invitrogen #11360-070), 2 mM L-glutamine (Invitrogen #25030-081) and Penicillin-Streptomycin (Invitrogen #15140-122). The cells will be washed 2 times with supplemented DMEM medium (ATCC #30-2002; supplemented with 0.1% BSA, 10 mM HEPES pH 7 and 1 mM MnCl) then re-suspended in supplemented DMEM medium at a density of 4×10 cells/mL. B750 μL of rh MAdCAM-1 (R&D systems #6065-MC) at 200 ng/well in 1×PBS was added to a Nunc MaxiSorp plate and incubated at 4° C. overnight. The solution was will be and replaced with 250 μL/well of block (PBS containing 1% BSA), then incubated at 37° C. for 1 hour. The solution will be removed. Compounds will be diluted by serial dilution in a final volume of 50 μL/well (2× concentration). To each well, 50 μL of cells (200,000 cells) will be added and the plate will be incubated at 37° C., 5% CO, for 30-45 minutes. The wells were washed 3 times with 100 μl/well of supplemented DMEM. Following the final wash, 100 μl/well of supplemented DMEM and 10 μl/well of MTT reagent (ATTC #30-1010K) will be added, and the plate will be incubated at 37° C., 5% C₀₂ for 2-3 hours. When a purple precipitate is visible, Detergent Reagent (ATTC #30-1010K) will be added at 100 μL/well. The plate will be left overnight at room temperature protected from light and wrapped in Parafilm. On the next day, the plate will be shaken for 5 minutes and the absorbance will be read at 570 nm.

### α4β7-MadCAM Competition ELISA

Another competition ELISA was performed by coating a 96 well plate with 100 μL of 4 μg/mL rh-integrin α4β7/LPAM-1 for one hour at 28° C. while shaking. After washing with wash buffer, 200 μL assay buffer was added to each well and incubated for one hour at 28° C. After another wash with wash buffer, 100 μL of a MAdCAM and peptide mixture in assay buffer was added to each well and incubated for one hour at 28° C., followed by washing with wash buffer. 50 μL of anti-human IgG1 diluted at 1:2000 in assay buffer was added, followed by incubation at 25° C. while shaking, washing with wash buffer, and adding 100 μL TMB solution. After the reaction was stopped, absorbance was measured at 450 nm. Results are shown in Table 6.

TABLE 6
IC50

| Sequence | IC50 (uM) |
|---|---|
| SEQ ID NO: 19 | >500 |
| SEQ ID NO: 49 | >500 |
| SEQ ID NO: 71 | >500 |
| SEQ ID NO: 72 | >500 |
| SEQ ID NO: 73 | >500 |
| SEQ ID NO: 74 | 499.3 |
| SEQ ID NO: 75 | 341.48 |
| SEQ ID NO: 10 | 310.62 |
| SEQ ID NO: 76 | 263.4 |
| SEQ ID NO: 77 | 177.5 |
| SEQ ID NO: 78 | 167.4 |
| SEQ ID NO: 79 | 162.1 |
| SEQ ID NO: 80 | 152.3 |
| SEQ ID NO: 8 | 151.4 |
| SEQ ID NO: 81 | 127.2 |
| SEQ ID NO: 82 | 124.5 |
| SEQ ID NO: 83 | 113.8 |
| SEQ ID NO: 70 | 111.5 |
| SEQ ID NO: 39 | 111.5 |
| SEQ ID NO: 84 | 100.08 |
| SEQ ID NO: 30 | 93.22 |
| SEQ ID NO: 85 | 79.77 |
| SEQ ID NO: 86 | 79.31 |
| SEQ ID NO: 16 | 72.51 |
| SEQ ID NO: 87 | 71.31 |
| SEQ ID NO: 88 | 70.71 |
| SEQ ID NO: 11 | 66.63 |
| SEQ ID NO: 28 | 65.7 |
| SEQ ID NO: 7 | 63.83 |
| SEQ ID NO: 12 | 59.12 |
| SEQ ID NO: 56 | 55.92 |
| SEQ ID NO: 89 | 55.9 |
| SEQ ID NO: 29 | 53.46 |
| SEQ ID NO: 48 | 43.71 |
| SEQ ID NO: 55 | 41.13 |
| SEQ ID NO: 90 | 34.3 |
| SEQ ID NO: 91 | 31.32 |
| SEQ ID NO: 53 | 31 |
| SEQ ID NO: 92 | 30.62 |
| SEQ ID NO: 93 | 29.38 |
| SEQ ID NO: 27 | 20.18 |
| SEQ ID NO: 94 | 19.66 |
| SEQ ID NO: 95 | 17.69 |
| SEQ ID NO: 96 | 15.9 |
| SEQ ID NO: 97 | 15.7 |
| SEQ ID NO: 98 | 14.42 |
| SEQ ID NO: 62 | 13.81 |
| SEQ ID NO: 99 | 10.7 |
| SEQ ID NO: 100 | 7.95 |
| SEQ ID NO: 61 | 6.419 |
| SEQ ID NO: 101 | 5.981 |
| SEQ ID NO: 102 | 5.432 |
| SEQ ID NO: 54 | 4.015 |
| SEQ ID NO: 103 | 4.01 |
| SEQ ID NO: 104 | 3.895 |
| SEQ ID NO: 105 | 3.89 |
| SEQ ID NO: 106 | 3.45 |
| SEQ ID NO: 107 | 3.063 |
| SEQ ID NO: 108 | 2.7 |
| SEQ ID NO: 50 | 2.486 |
| SEQ ID NO: 59 | 1.922 |
| SEQ ID NO: 57 | 1.74 |
| SEQ ID NO: 109 | 1.731 |
| SEQ ID NO: 110 | 1.71 |
| SEQ ID NO: 111 | 1.696 |
| SEQ ID NO: 52 | 1.641 |
| SEQ ID NO: 112 | 1.614 |
| SEQ ID NO: 63 | 1.463 |
| SEQ ID NO: 113 | 1.45 |
| SEQ ID NO: 114 | 1.4 |
| SEQ ID NO: 115 | 1.299 |
| SEQ ID NO: 116 | 1.27 |
| SEQ ID NO: 117 | 1.13 |
| SEQ ID NO: 118 | 1.042 |
| SEQ ID NO: 46 | 0.943 |
| SEQ ID NO: 119 | 0.932 |
| SEQ ID NO: 120 | 0.796 |
| SEQ ID NO: 121 | 0.748 |
| SEQ ID NO: 122 | 0.740 |
| SEQ ID NO: 123 | 0.729 |
| SEQ ID NO: 26 | 0.723 |
| SEQ ID NO: 124 | 0.690 |
| SEQ ID NO: 125 | 0.672 |
| SEQ ID NO: 126 | 0.672 |
| SEQ ID NO: 127 | 0.620 |
| SEQ ID NO: 128 | 0.602 |
| SEQ ID NO: 152 | 0.552 |
| SEQ ID NO: 129 | 0.517 |
| SEQ ID NO: 130 | 0.511 |
| SEQ ID NO: 131 | 0.495 |
| SEQ ID NO: 132 | 0.450 |
| SEQ ID NO: 133 | 0.404 |
| SEQ ID NO: 134 | 0.363 |
| SEQ ID NO: 135 | 0.345 |
| SEQ ID NO: 136 | 0.307 |
| SEQ ID NO: 137 | 0.272 |
| SEQ ID NO: 138 | 0.261 |
| SEQ ID NO: 139 | 0.260 |
| SEQ ID NO: 140 | 0.227 |
| SEQ ID NO: 141 | 0.204 |
| SEQ ID NO: 142 | 0.198 |
| SEQ ID NO: 143 | 0.188 |
| SEQ ID NO: 144 | 0.187 |
| SEQ ID NO: 69 | 0.186 |
| SEQ ID NO: 151 | 0.136 |
| SEQ ID NO: 145 | 0.130 |
| SEQ ID NO: 146 | 0.122 |
| SEQ ID NO: 147 | 0.075 |
| SEQ ID NO: 148 | 0.053 |
| SEQ ID NO: 149 | 0.033 |
| SEQ ID NO: 150 | 0.003 |

Those skilled in the art to which the present disclosure pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present invention has been described with reference to particular embodiments, modifications of structure, sequence, materials and the like apparent to those skilled in the art still fall within the scope as claimed.

1. A compound comprising a peptide scaffold and one or more of a linker, a sulfide or disulfide bond, wherein the linker, sulfide, or disulfide bond is covalently bonded to the peptide scaffold.

2. The compound of claim 1, wherein the peptide scaffold comprises a sequence of Formula (I):
XY¹Y²X′ₘZ¹Z²Z³Z⁴Z⁵
or a pharmaceutically acceptable salt thereof, wherein
X is Ser, Cys, Gly, Arg, R(NMe), Ac-Ser, or Ac-Cys;
Y¹ is any amino acid;
Y² is Cys, Ser, or Asp;
each X independently is any amino acid;
Z¹ is Cys, Asp, Thr, or absent;
Z² is Leu, Ile, Val, Lys, or absent;
Z³ is Cys or absent;
Z⁴ is Trp or absent;
Z⁵ is Gln or absent; and
m is 4, 5, 6, or 7 and each X may be the same or different.

3. The compound of claim 2, wherein
Y¹ is Gln, Lys, His, Thr, Glu, Cys, Phe, Met, Asp, Ala, Arg, Ile, Asn, Gly, Leu, Ser, or Trp;
each X independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen.

4. (canceled)

5. The compound of claim 1, wherein the peptide scaffold comprises a sequence of Formula (IV):
XY¹Y²X′ₘZ¹
or a pharmaceutically acceptable salt thereof, wherein
X is Ser, Cys, Gly, R(NMe), or Ac-Cys;
Y¹ is Lys, Arg, or Ser;
Y² is Cys, Ser, or Asp;
each X independently is Pro, Asp, Met, Leu, Thr, Nle, Hse(OMe), Ile, Val, Pen, Cys, Trp, Ala, Gly, Tyr, Gln, Glu, Lys, or absent;
Z¹ is Cys or absent; and
m is 4, 5, 6, or 7 and each X may be the same or different.

6. The compound of claim 1, wherein the linker comprises DIG, PEG, IDA, ADA, Boc-IDA, Glutaric acid, Isopthalic acid, 1,3-phenylenediacetic acid, 1,4-phenylenediacetic acid, 1,2-phenylenediacetic acid, Triazine, Boc-Triazine, IDA-biotin, PEG4-biotin, AADA, any suitable aliphatics, aromatics, heteroaromatics, polyethylene glycol, two-fold symmetric linchpin (TSL), TSLC, PFS, TBMB, DBMB, chloroacetic acid, 3-chloropropanoic acid, or 2-(chloromethyl)benzoic acid.

7. (canceled)

8. The compound of claim 2, wherein the X is Ser; the linker is a TSL; and wherein the linker is bound to the peptide scaffold at the Ser by an aldehyde reactive oxime functionality.

9. The compound of claim 2, wherein the X is Gly; the linker is a TSLC; and wherein the linker is bound to the peptide scaffold at the Gly by an amide functionality.

10. The compound of claim 2, wherein the X is R(NMe); the linker is 3-chloropropanoic acid (Pa) or 2-(chloromethyl)benzoic acid (Bz); and wherein the linker is bound to the peptide scaffold at the R(NMe) by an amide functionality.

11. The compound of claim 2, wherein the X is R; the linker is chloroacetic acid; and wherein the linker is bound to the peptide scaffold at the R by an amide functionality.

12.-20. (canceled)

21. A compound selected from the group consisting of scaffold A, B, C, D, E, F, G, H I, and J, wherein
each X independently is Ser, Cys, Gly, Arg, R(NMe), Ac-Ser, or Ac-Cys;
each Y¹ independently is any amino acid;
each Y² independently is any amino acid;
each X independently is any amino acid;
each X″ independently is any amino acid or absent;
each C″ independently is Cys or an Ac-Cys;
each X″ independently is any amino acid;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

22. The compound of claim 21, wherein
each X independently is Ser, Cys, Gly, Arg, R(NMe), Ac-Ser, or Ac-Cys;
each Y¹ independently is Gln, Lys, His, Thr, Glu, Cys, Phe, Met, Asp, Ala, Arg, Ile, Asn, Gly, Leu, Ser, or Trp;
each Y² independently is Cys, Ser, or Asp;
each X independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;
each X″ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, Pen, or absent;
each C″ independently is Cys or an Ac-Cys;
each X″ independently is Ser, Cys, Gly, Arg, R(NMe), Ac-Ser, or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent; and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

23. The compound of claim 21, wherein
each X independently is Ser, Cys, Gly, Arg, R(NMe), Ac-Ser, or Ac-Cys;
each Y¹ independently is Lys, Arg, or Ser;
each Y² independently is Cys, Ser, or Asp;
each X independently is Pro, Asp, Met, Leu, Thr, Nle, Hse(OMe), Ile, Val, Pen, Cys, Trp, Ala, Gly, Tyr, Gln, Glu, Lys, or absent;
each X″ independently is Pro, Asp, Met, Leu, Thr, Nle, Hse(OMe), Ile, Val, Pen, Cys, Trp, Ala, Gly, Tyr, Gln, Glu, Lys, or absent;
each C″ independently is Cys or an Ac-Cys;
each X″ independently is Ser, Cys, Gly, R(NMe), or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

24. A compound selected from the group consisting of scaffold A-S, B-S, C-S, D-S, E-S, F-S, G-S, H-S, I-S, and J-S, wherein
each X independently is Ser, Cys, Gly, Arg, R(NMe), Ac-Ser, or Ac-Cys;
each Y¹ independently is any amino acid;
each Y² independently is any amino acid;
each X independently is any amino acid;
each X″ independently is any amino acid or absent;
each C″ independently is Cys or an Ac-Cys;
each X″ independently is any amino acid;
each Z¹ independently is Cys, Asp, or Thr;
each Z² independently is Leu, Ile, Val, or Lys;
each Z⁴ independently is Trp or absent;
each Z⁵ independently is Gln or absent; and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

25. The compound of claim 24, wherein
each X independently is Ser, Cys, Gly, Arg, R(NMe), Ac-Ser, or Ac-Cys;
each Y¹ independently is Gln, Lys, His, Thr, Glu, Cys, Phe, Met, Asp, Ala, Arg, Ile, Asn, Gly, Leu, Ser, or Trp;
each Y² independently is Cys, Ser, or Asp;
each X independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, or Pen;
each X″ independently is His, Pro, Lys, Arg, Phe, Nle, Gln, Tyr, Gly, Leu, Trp, Asp, Ser, Met, Ala, Ile, Thr, Ox-Met, Hse(OMe), Glu, tBuAla, CbA, Asn, Val, Cys, Pen, or absent;
each C″ independently is Cys or an Ac-Cys;
each X″ independently is Ser, Cys, Gly, Arg, R(NMe), Ac-Ser, or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent; and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

26. The compound of claim 24, wherein
each X independently is Ser, Cys, Gly, Arg, R(NMe), Ac-Ser, or Ac-Cys;
each Y¹ independently is Lys, Arg, or Ser;
each Y² independently is Cys, Ser, or Asp;
each X independently is Pro, Asp, Met, Leu, Thr, Nle, Hse(OMe), Ile, Val, Pen, Cys, Trp, Ala, Gly, Tyr, Gln, Glu, Lys, or absent;
each X″ independently is Pro, Asp, Met, Leu, Thr, Nle, Hse(OMe), Ile, Val, Pen, Cys, Trp, Ala, Gly, Tyr, Gln, Glu, Lys, or absent;
each C″ independently is Cys or an Ac-Cys;
each X″ independently is Ser, Cys, Gly, R(NMe), or Ac-Cys;
each Z¹ independently is Cys, Asp, or Thr;
each Z² independently is Leu, Ile, Val, or Lys;
each Z⁴ independently is Trp or absent;
each Z⁵ independently is Gln or absent;
and
each of R¹, R², R³, R⁴, R⁵, R⁶, R⁷, R⁸, R⁹, R¹⁰, R¹¹, R¹², R¹⁴ and R¹⁵ independently, is a corresponding side chain specific to each amino acid.

27. The compound of any one of claims 21-26, wherein the compound is bicyclic.

28. A compound selected from the group consisting of:

| No. | Structure |
|---|---|
| 1 | 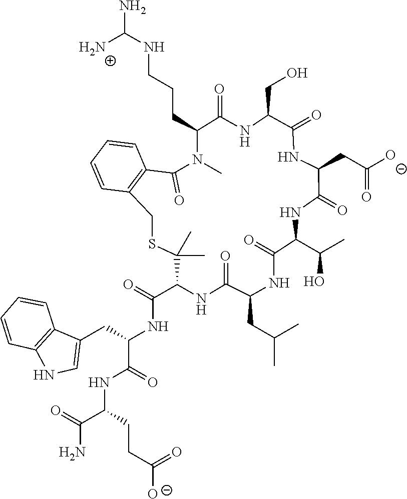 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)[NH3+])N(C)C(=O)c2ccccc2CSC(C)(C)[C@@H](C(=O)N[C@@H](Cc2c[nH]c3ccccc23)C(=O)N[C@H](CCC(=O)[O-])C(N)=O)NC1=O] |
| 2 | 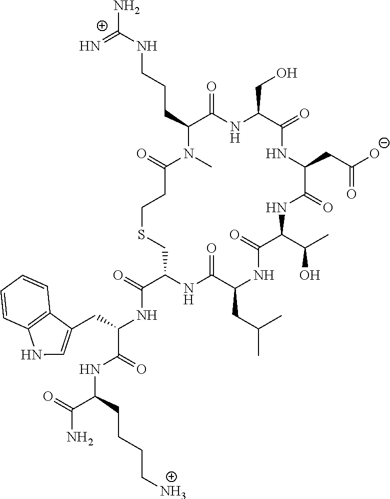 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])N(C)C(=O)CCSC[C@@H](C(=O)N[C@@H](Cc2cn(C)c3ccccc23)C(=O)N[C@@H](CCCC[NH3+])C(N)=O)NC1=O] |
| 3 | 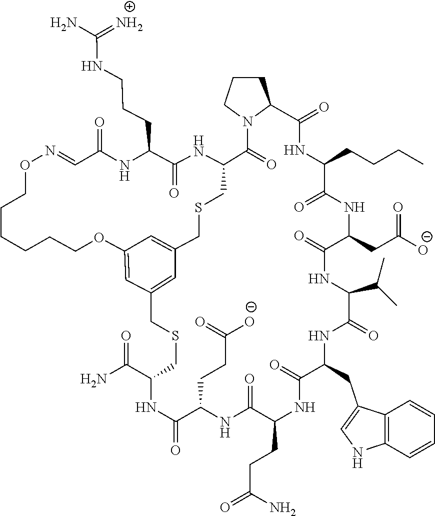 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 4 | 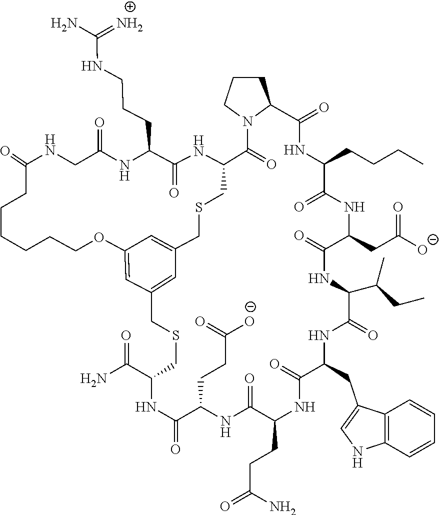 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 5 | 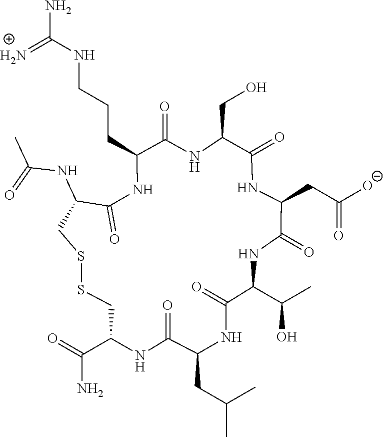 [SMILES: CC(=O)N[C@H]1CSSC[C@@H](C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 6 | 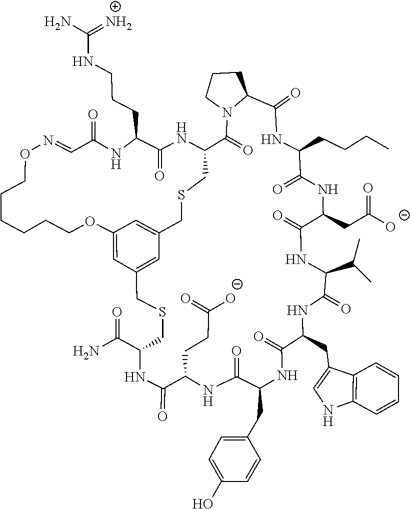 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC(=O)[C@H](C(C)C)NC(=O)[C@H](CC(=O)[O-])NC1=O] |
| 7 | 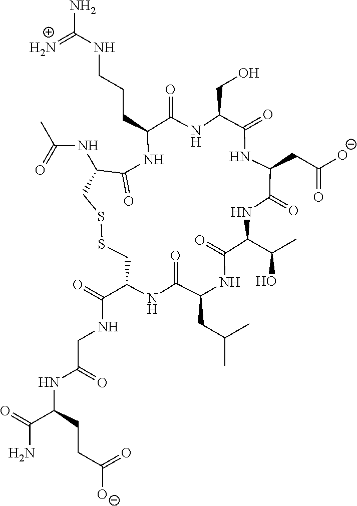 [SMILES: CC(=O)N[C@H]1CSSC[C@@H](C(=O)NCC(=O)N[C@@H](CCC(=O)[O-])C(N)=O)NC(=O)[C@H](CC(C)C)NC(=O)[C@H]([C@@H](C)O)NC(=O)[C@H](CC(=O)[O-])NC(=O)[C@H](CO)NC(=O)[C@H](CCCNC(N)=[NH2+])NC1=O] |
| 8 | 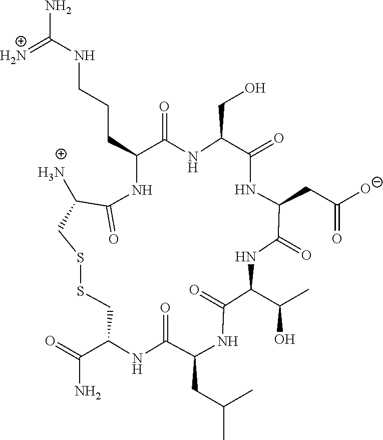 [SMILES: CC(C)C[C@@H]1NC(=O)[C@H]([C@@H](C)O)NC(=O)[C@H](CC(=O)[O-])NC(=O)[C@H](CO)NC(=O)[C@H](CCCNC(N)=[NH2+])NC(=O)[C@@H]([NH3+])CSSC[C@@H](C(N)=O)NC1=O] |
| 9 | 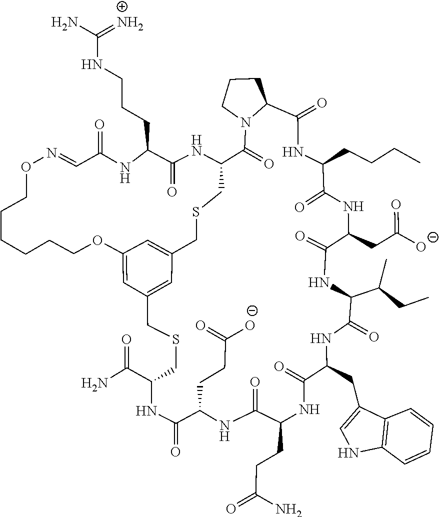 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H]([C@@H](C)CC)NC(=O)[C@H](CC(=O)[O-])NC1=O] |
| 10 | 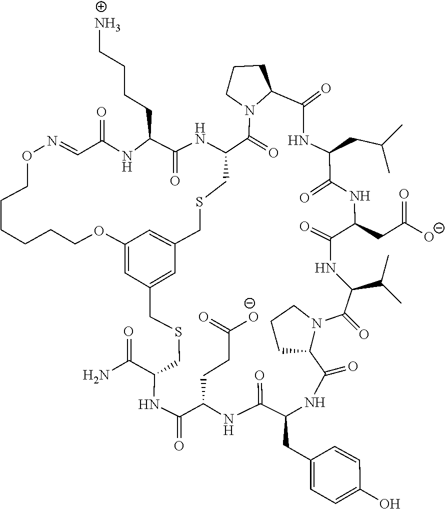 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C(C)C)NC(=O)[C@H](CC(=O)[O-])NC1=O] |
| 11 | 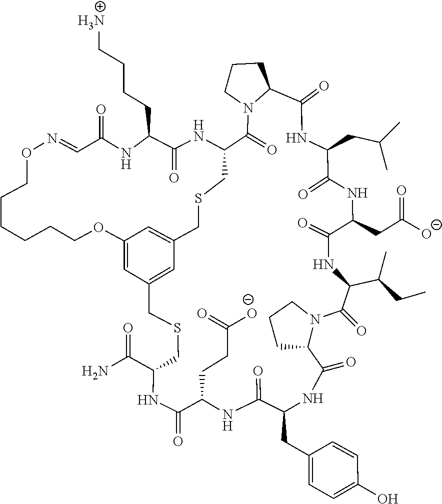 [SMILES: CC[C@H](C)[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 12 | 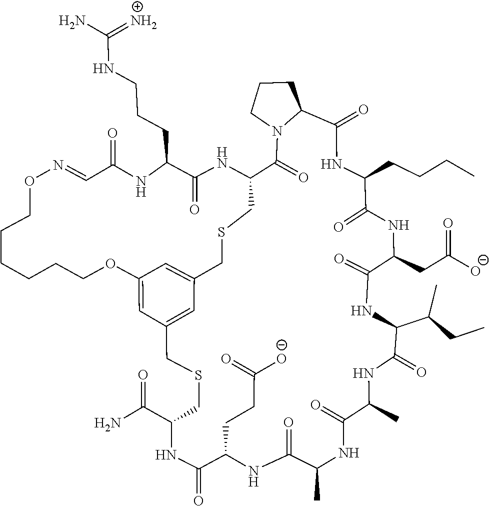 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](C)NC(=O)[C@H](C)NC(=O)[C@H]([C@@H](C)CC)NC(=O)[C@H](CC(=O)[O-])NC1=O] |
| 13 | 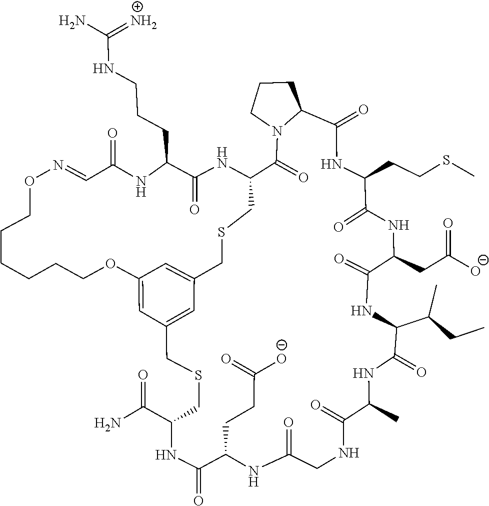 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC1=O] |
| 14 | 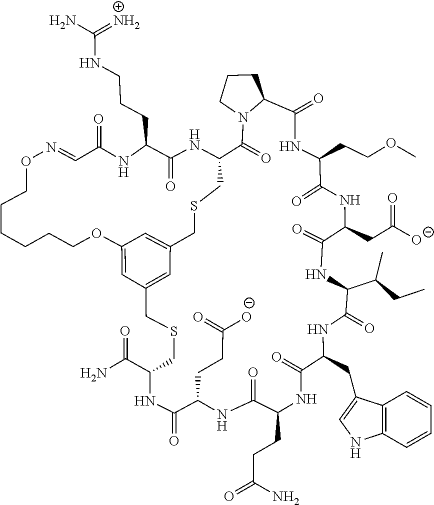 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 15 | 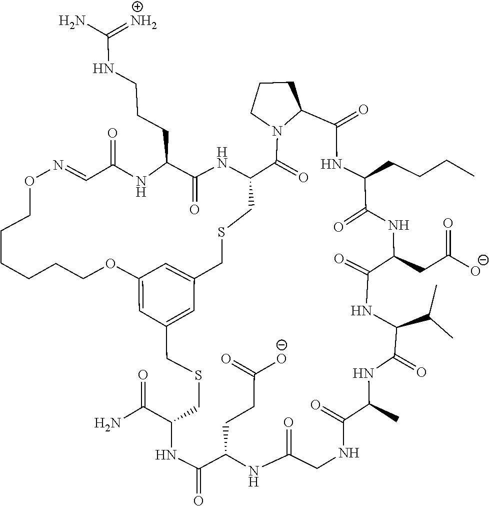 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 16 | 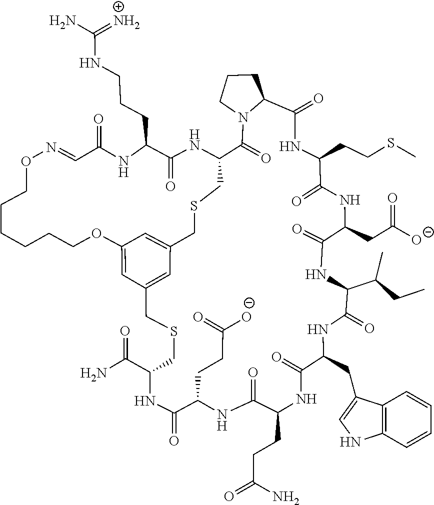 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 17 | 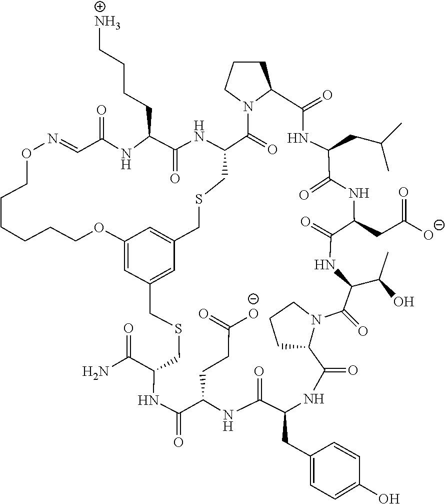 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 18 | 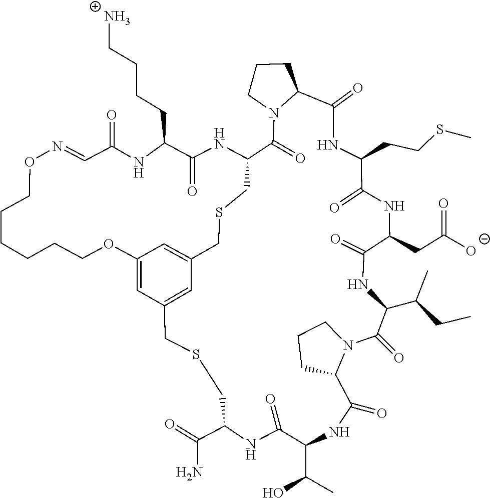 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H]([C@@H](C)O)NC(=O)[C@@H]2CCCN2C1=O] |
| 19 | 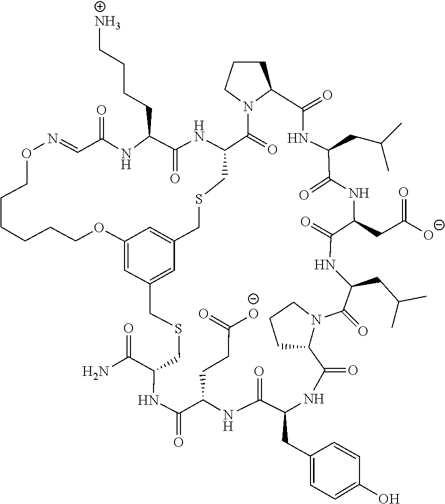 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](CC(C)C)NC(=O)[C@H](CC(=O)[O-])NC1=O] |
| 20 | 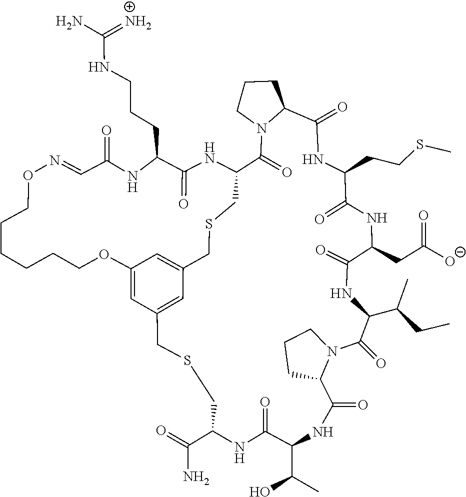 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H]([C@@H](C)O)NC(=O)[C@@H]2CCCN2C1=O] |
| 21 | 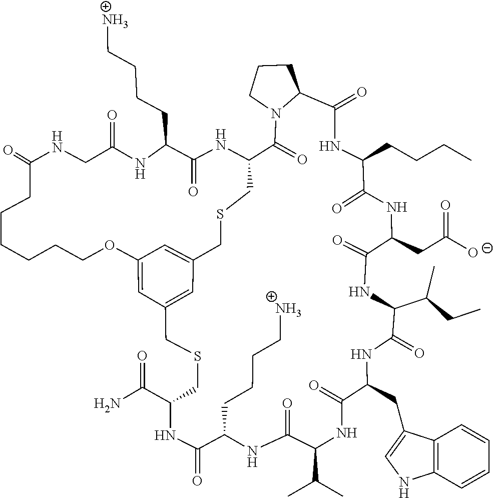 [SMILES: CCCC[C@@H]1NC(=O)[C@@H]2CCCN2C(=O)[C@@H]2CSCc3cc(cc(c3)OCCCCCCC(=O)NCC(=O)N[C@@H](CCCC[NH3+])C(=O)N2)CSC[C@@H](C(N)=O)NC(=O)[C@H](CCCC[NH3+])NC(=O)[C@H](C(C)C)NC(=O)[C@H](Cc2c[nH]c3ccccc23)NC(=O)[C@H]([C@@H](C)CC)NC(=O)[C@H](CC(=O)[O-])NC1=O] |
| 22 | 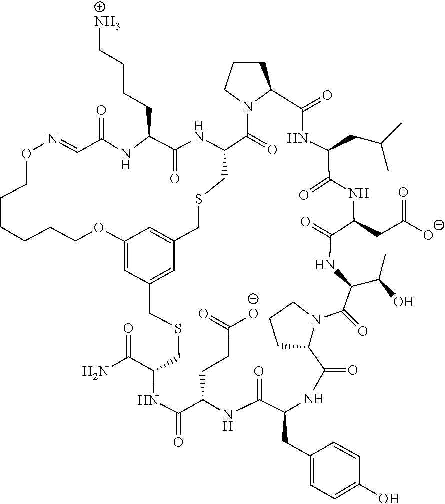 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 23 | 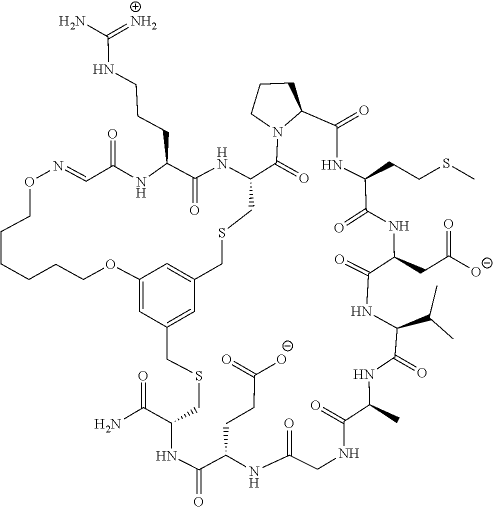 [SMILES: CSCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 24 | 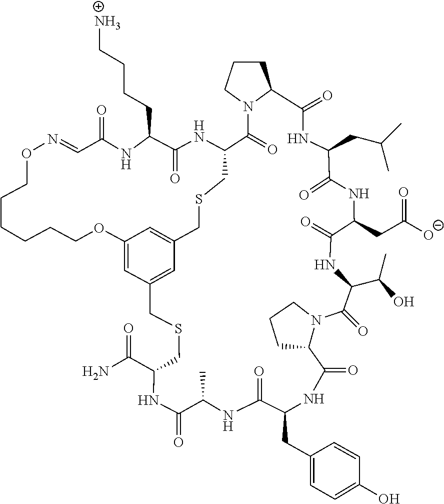 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 25 | 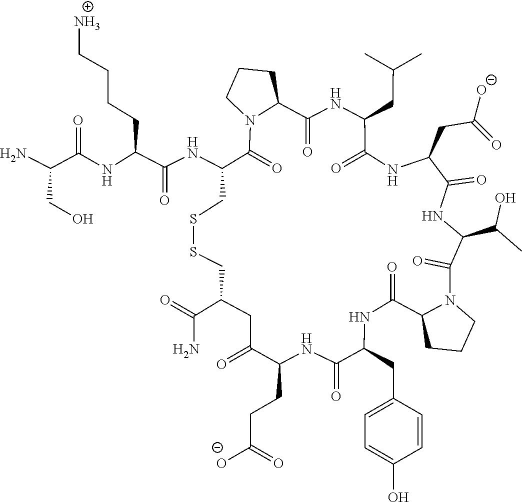 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)CC(=O)[C@H](CCC(=O)[O-])NC(=O)[C@H](Cc2ccc(O)cc2)NC(=O)[C@@H]2CCCN2C(=O)[C@H](C(C)O)NC(=O)[C@H](CC(=O)[O-])NC1=O] |
| 26 | 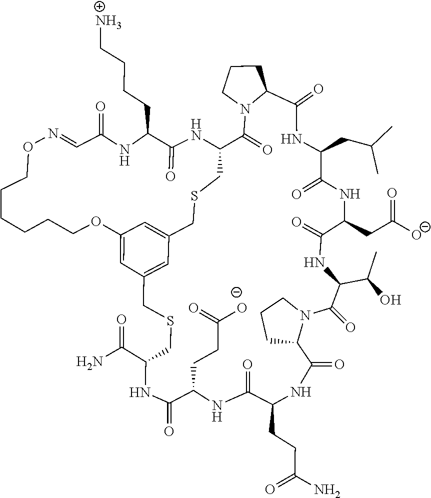 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](CCC(N)=O)NC(=O)[C@@H]2CCCN2C(=O)[C@H]([C@@H](C)O)NC(=O)[C@H](CC(=O)[O-])NC1=O] |
| 27 | 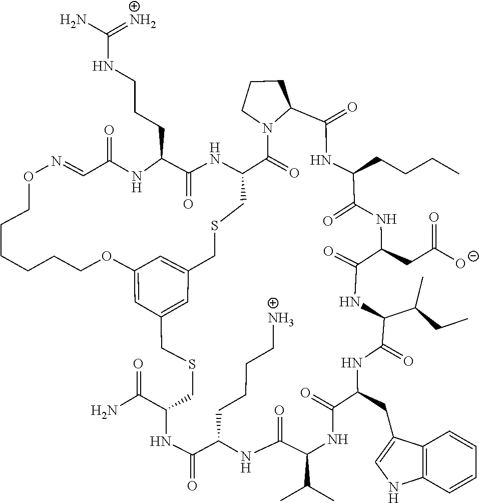 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCCC[NH3+])NC(=O)[C@H](C(C)C)NC(=O)[C@H](Cc2c[nH]c3ccccc23)NC(=O)[C@H]([C@@H](C)CC)NC(=O)[C@H](CC(=O)[O-])NC1=O] |
| 28 | 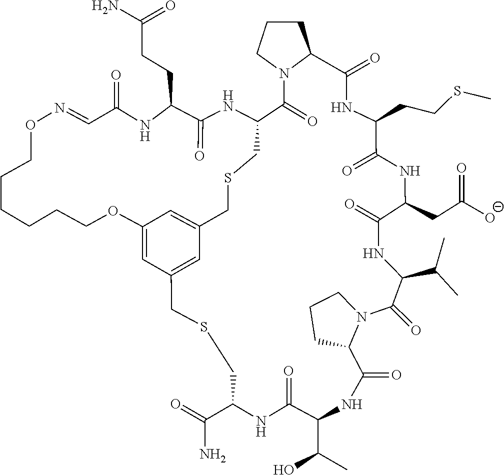 [SMILES: CSCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C(=O)[C@H](C(C)C)NC(=O)[C@H](CC(=O)[O-])NC1=O] |
| 29 | 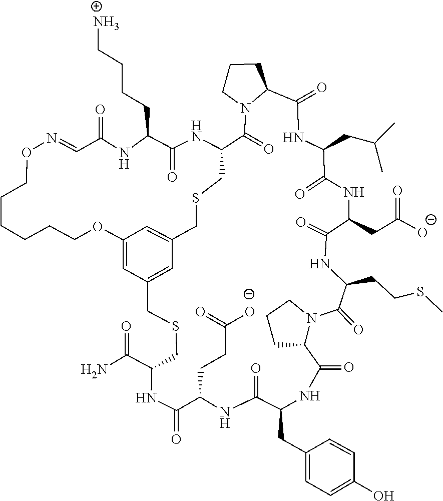 [SMILES: CSCC[C@@H]1NC(=O)[C@H](CC(=O)[O-])NC(=O)[C@H](CC(C)C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C1=O] |
| 30 | 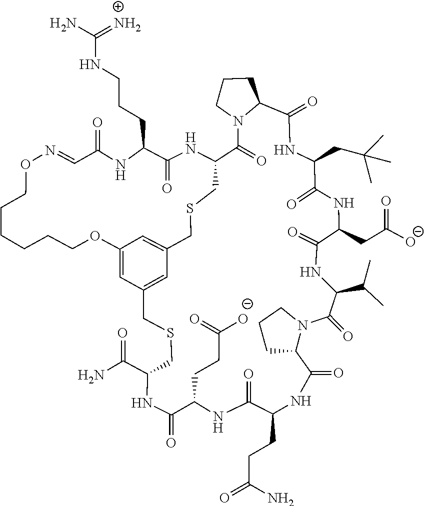 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@@H]2CCCN2C1=O] |
| 31 | 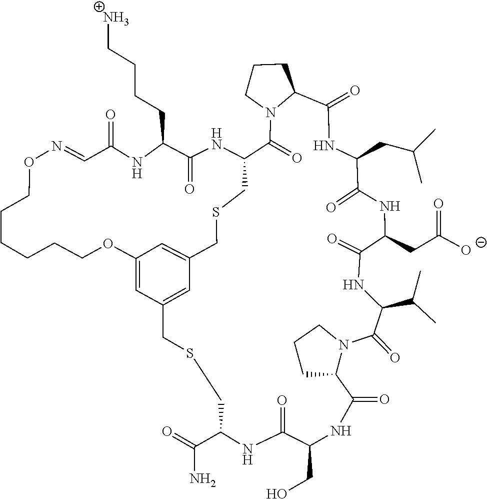 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 32 | 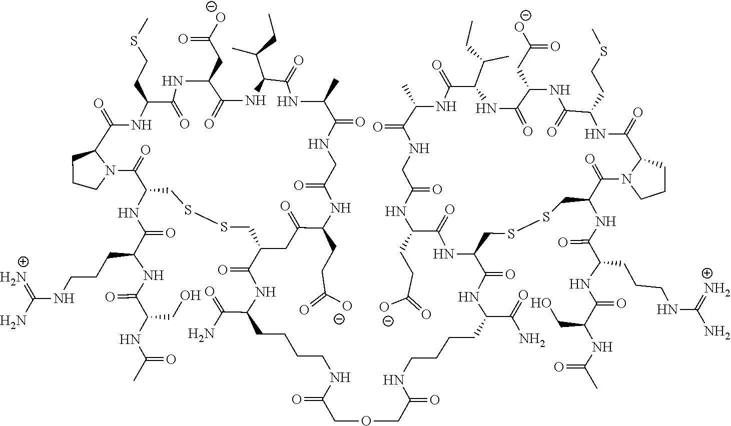 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(=O)N[C@@H](CCCCNC(=O)COCC(=O)NCCCC[C@H](NC(=O)[C@@H]2CSSC[C@H](NC(=O)[C@H](CCCNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)CC(=O)[C@H](CCC(=O)[O-])NC(=O)CNC(=O)[C@H](C)NC1=O] |
| 33 | 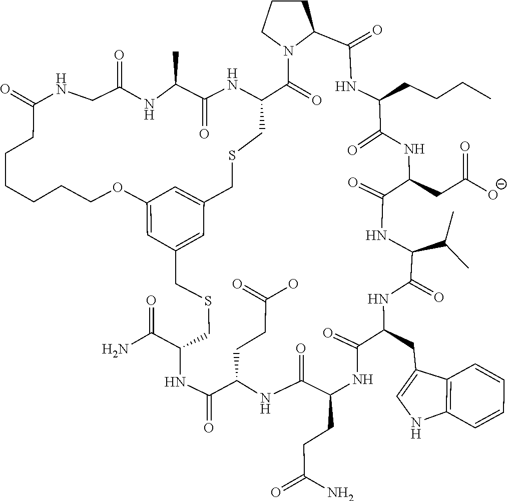 |
| 34 | 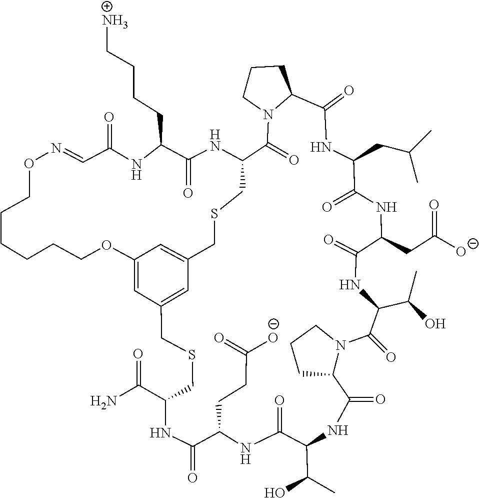 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H]([C@@H](C)O)NC(=O)[C@@H]2CCCN2C(=O)[C@H]([C@@H](C)O)NC(=O)[C@H](CC(=O)[O-])NC1=O] |
| 35 | 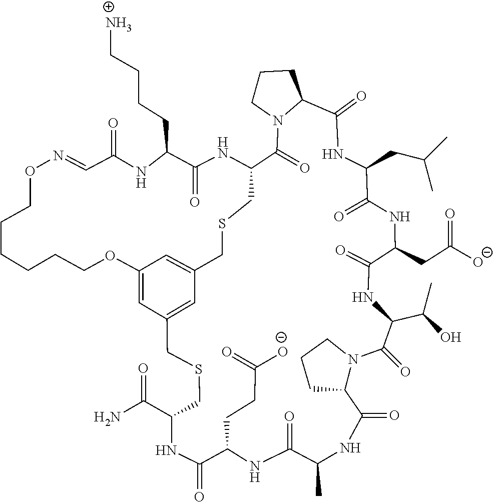 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](C)NC(=O)[C@@H]2CCCN2C(=O)[C@H]([C@@H](C)O)NC(=O)[C@H](CC(=O)[O-])NC1=O] |
| 36 | 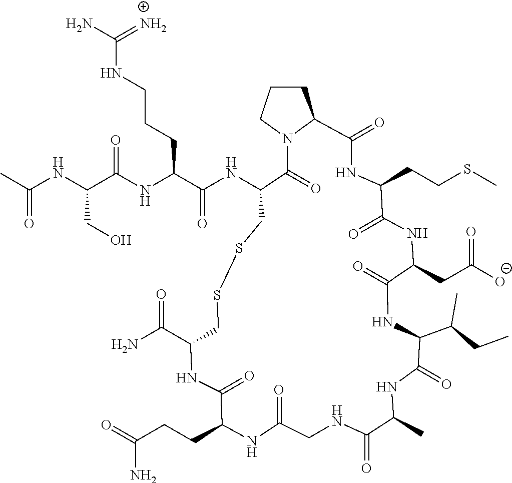 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(N)=O)NC(=O)[C@H](CCC(N)=O)NC(=O)CNC(=O)[C@H](C)NC1=O] |
| 37 | 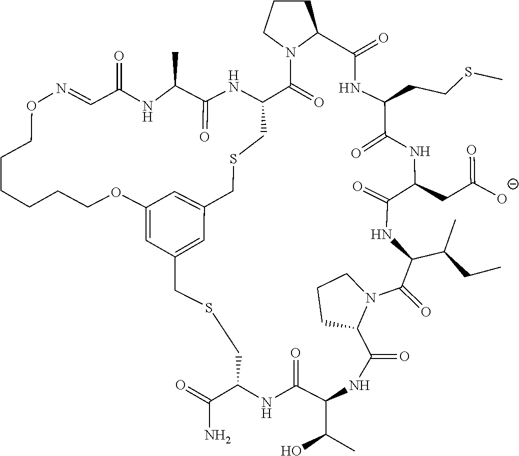 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H]([C@@H](C)O)NC(=O)[C@@H]2CCCN2C1=O] |
| 38 | 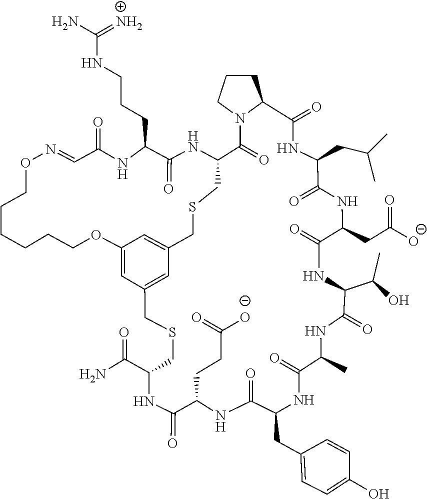 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](C)NC(=O)[C@H]([C@@H](C)O)NC(=O)[C@H](CC(=O)[O-])NC1=O] |
| 39 | 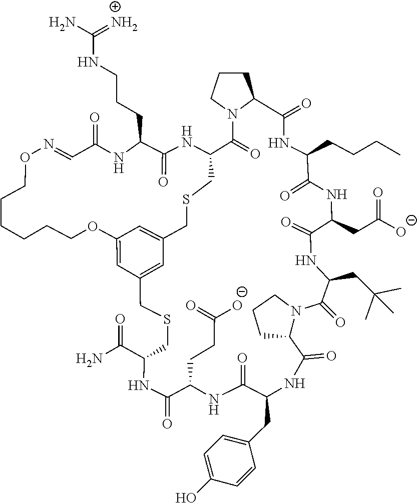 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](CC(C)(C)C)NC(=O)[C@H](CC(=O)[O-])NC1=O] |
| 40 | 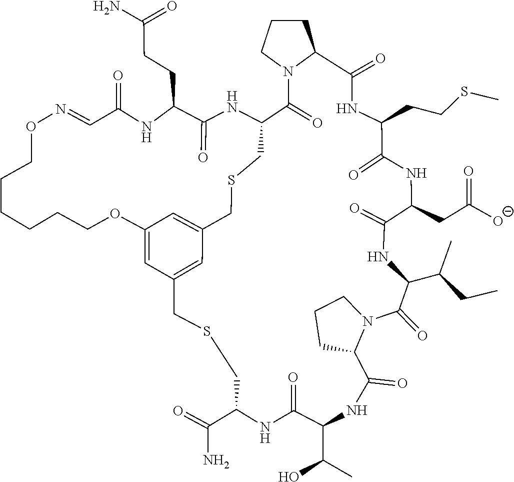 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C1=O] |
| 41 | 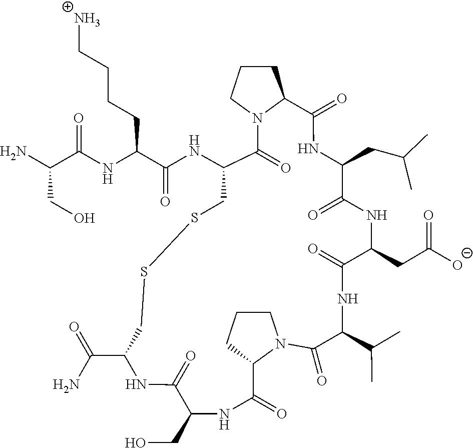 [SMILES: CC(C)C[C@@H]1NC(=O)[C@@H]2CCCN2C(=O)[C@@H](NC(=O)[C@H](CCCC[NH3+])NC(=O)[C@@H](N)CO)CSSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 42 | 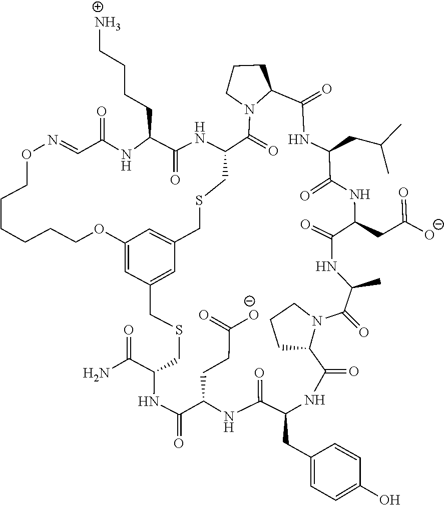 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H](C)NC(=O)[C@H](CC(=O)[O-])NC1=O] |
| 43 | 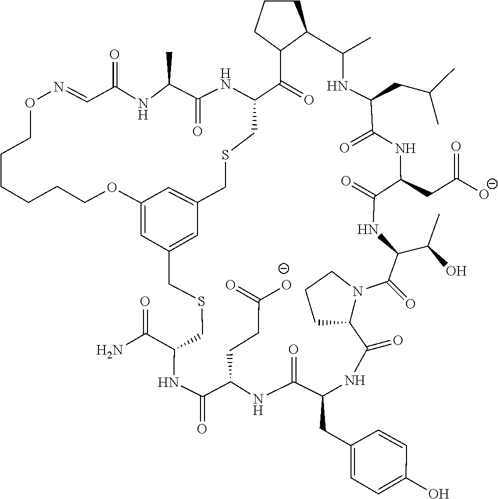 |
| 44 | 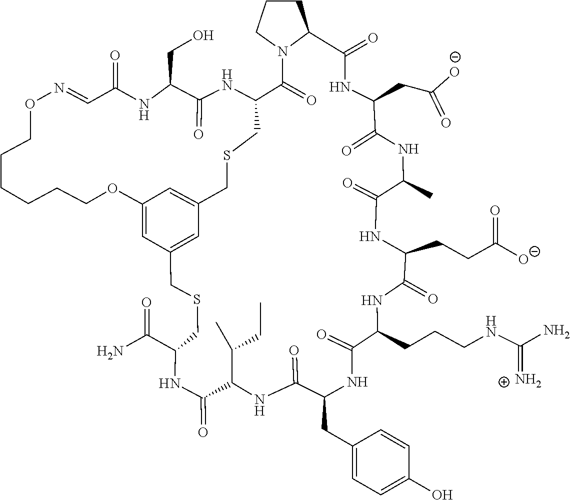 |
| 45 | 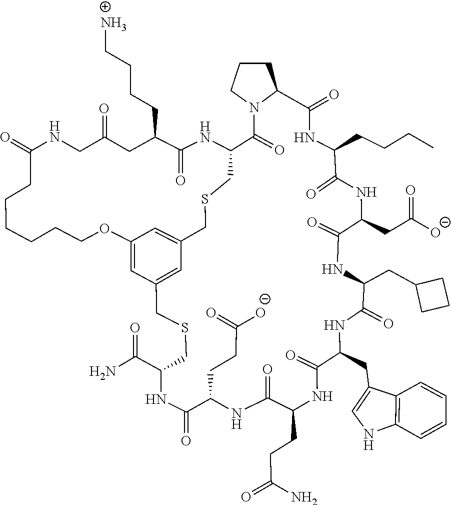 [SMILES: CCCC[C@@H]1NC(=O)[C@@H]2CCCN2C(=O)[C@@H]2CSCc3cc(cc(c3)OCCCCCCC(=O)NCC(=O)C[C@@H](CCCCN)C(=O)N2)CSC[C@@H](C(N)=O)NC(=O)[C@H](CCC(=O)O)NC(=O)[C@H](CCC(N)=O)NC(=O)[C@H](Cc2c[nH]c3ccccc23)NC(=O)[C@H](CC2CCC2)NC(=O)[C@H](CC(=O)[O-])NC1=O] |
| 46 | 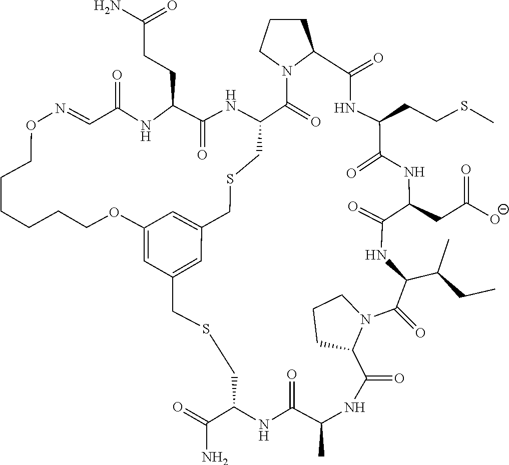 [SMILES: CC[C@H](C)[C@@H]1NC(=O)[C@H](CC(=O)[O-])NC(=O)[C@H](CCSC)NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H](C)NC(=O)[C@@H]2CCCN2C1=O] |
| 47 | 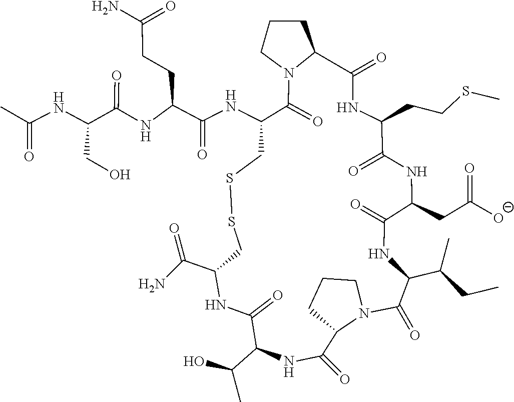 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCC(N)=O)NC(=O)[C@H](CO)NC(C)=O)CSSC[C@@H](C(N)=O)NC(=O)[C@H]([C@@H](C)O)NC(=O)[C@@H]2CCCN2C1=O] |
| 48 | 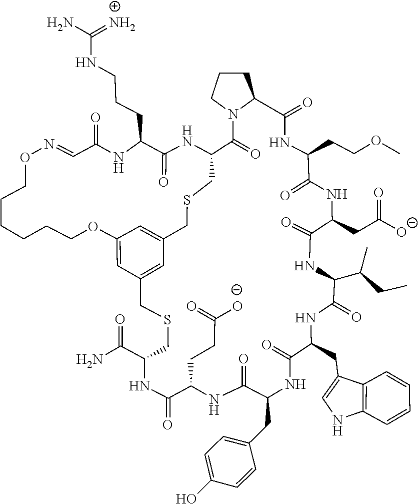 [SMILES: CC[C@H](C)[C@@H]1NC(=O)[C@H](CC(=O)[O-])NC(=O)[C@H](CCOC)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](Cc2ccc(O)cc2)NC(=O)[C@H](Cc2c[nH]c3ccccc23)NC1=O] |
| 49 | 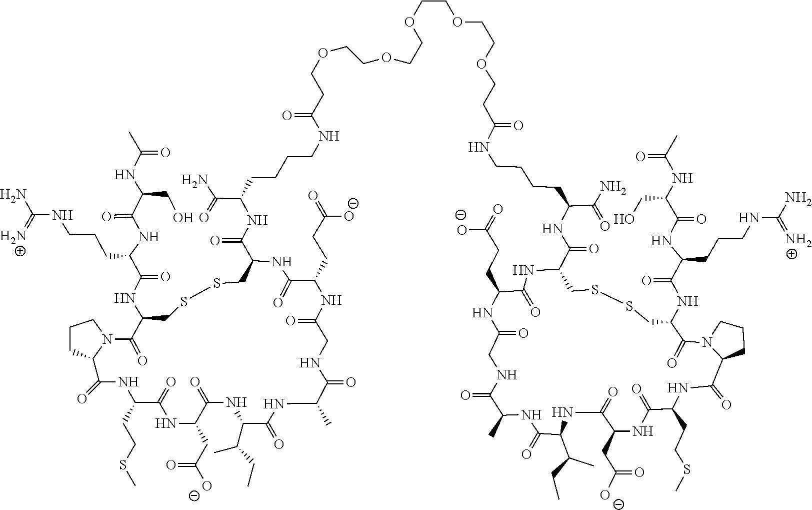 [SMILES: CC[C@H](C)[C@@H]1NC(=O)[C@H](CC(=O)[O-])NC(=O)[C@H](CCSC)NC(=O)[C@@H]2CCCN2C(=O)[C@@H](NC(=O)[C@H](CCCNC(N)=[NH2+])NC(=O)[C@H](CO)NC(C)=O)CSSC[C@@H](C(=O)N[C@@H](CCCCNC(=O)CCOCCOCCOCCOCCOCCC(=O)NCCCC[C@H](NC(=O)[C@@H]2CSSC[C@H](NC(=O)[C@H](CCCNC(N)=[NH2+])NC(=O)[C@H](CO)NC(C)=O)C(=O)N3CCC[C@H]3C(=O)N[C@@H](CCSC)C(=O)N[C@@H](CC(=O)[O-])C(=O)N[C@@H]([C@@H](C)CC)C(=O)N[C@@H](C)C(=O)NCC(=O)N[C@@H](CCC(=O)[O-])C(=O)N2)C(N)=O)C(N)=O)NC(=O)[C@H](CCC(=O)[O-])NC(=O)CNC(=O)[C@H](C)NC1=O] |
| 50 | 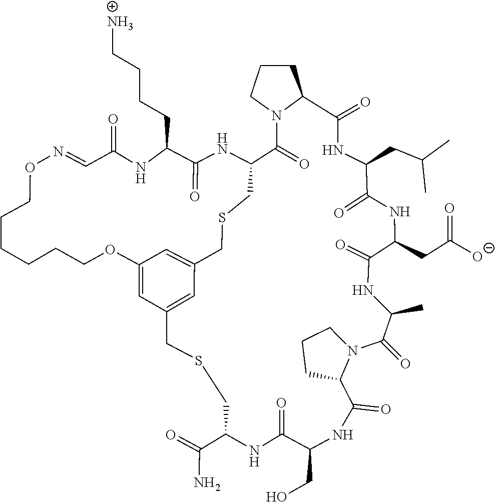 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C)NC(=O)[C@H](CC(=O)[O-])NC1=O] |
| 51 | 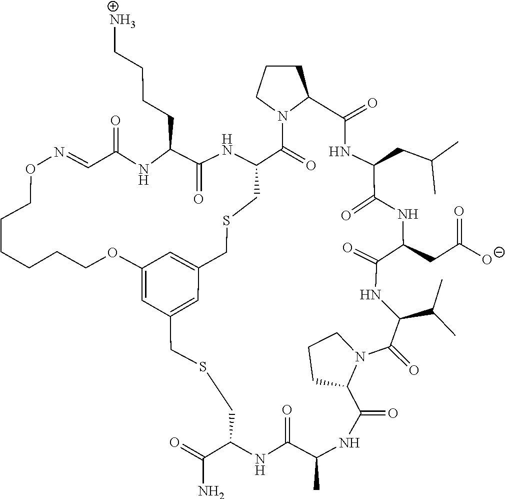 [SMILES: CC(C)C[C@@H]1NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](C)NC(=O)[C@@H]2CCCN2C(=O)[C@H](C(C)C)NC(=O)[C@H](CC(=O)[O-])NC1=O] |
| 52 | 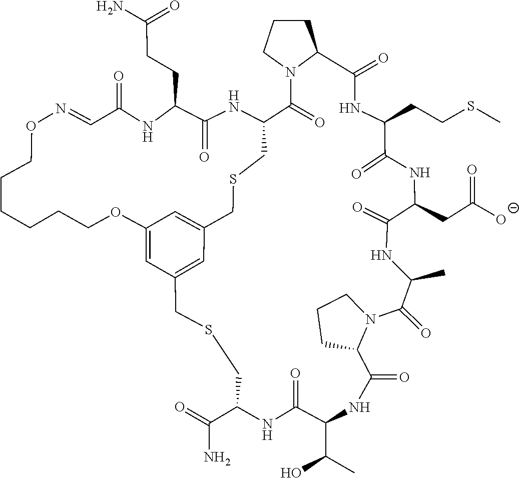 [SMILES: CSCC[C@@H]1NC(=O)[C@@H]2CCCN2C(=O)[C@@H]2CSCc3cc(cc(c3)OCCCCCCO/N=C/C(=O)N[C@@H](CCC(N)=O)C(=O)N2)CSC[C@@H](C(N)=O)NC(=O)[C@H]([C@@H](C)O)NC(=O)[C@@H]2CCCN2C(=O)[C@H](C)NC(=O)[C@H](CC(=O)[O-])NC1=O] |
| 53 | 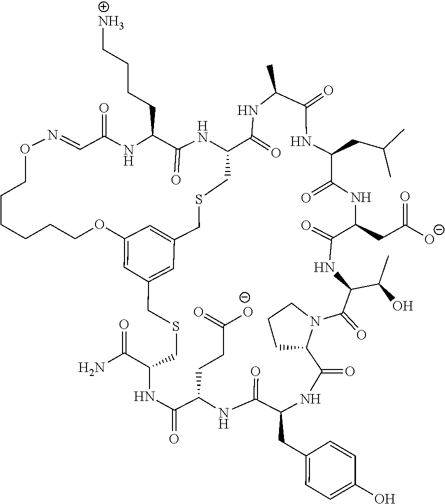 [SMILES: CC(C)C[C@@H]1NC(=O)[C@H](C)NC(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CCC(=O)[O-])NC(=O)[C@H](Cc2ccc(O)cc2)NC(=O)[C@@H]2CCCN2C(=O)[C@H]([C@@H](C)O)NC(=O)[C@H](CC(=O)[O-])NC1=O] |
| 54 | 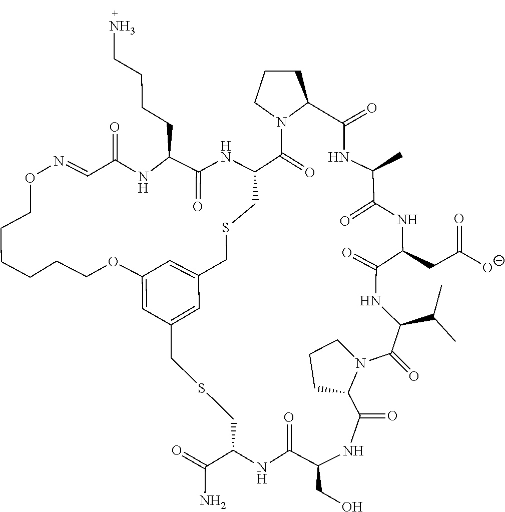 [SMILES: CC(C)[C@@H]1NC(=O)[C@H](CC(=O)[O-])NC(=O)[C@H](C)NC(=O)[C@@H]2CCCN2C(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C1=O] |
| 55 | 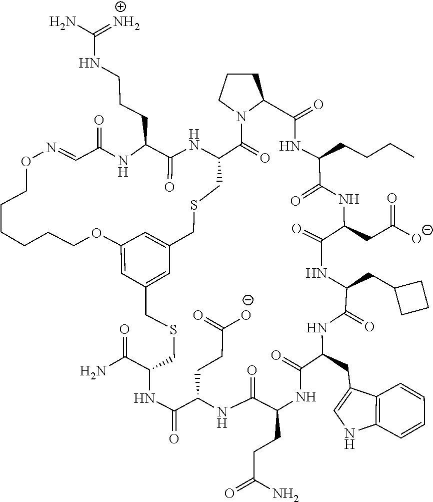 [SMILES: CCCC[C@@H]1NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](CC2CCC2)NC(=O)[C@H](CC(=O)[O-])NC1=O] |
| 56 | 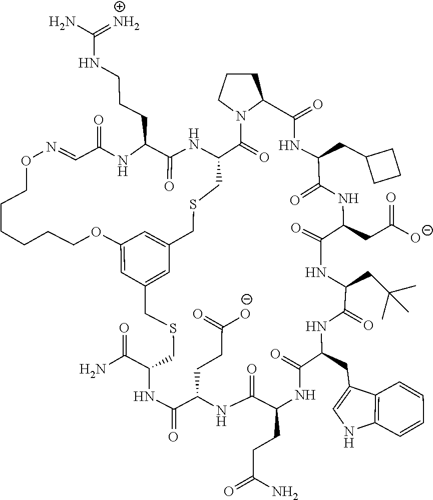 [SMILES: CC(C)(C)C[C@@H]1NC(=O)[C@H](CC(=O)[O-])NC(=O)[C@H](CC2CCC2)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC1=O] |
| 57 | 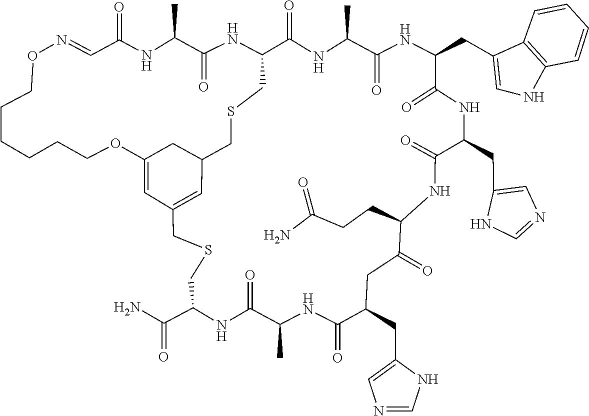 [SMILES: C[C@@H]1NC(=O)/C=N/OCCCCCCOC2=CC3=CC(CSC[C@H](NC1=O)C(=O)N[C@@H](C)C(=O)N[C@@H](Cc1c[nH]c4ccccc14)C(=O)N[C@@H](Cc1cnc[nH]1)C(=O)N[C@H](CCC(N)=O)C(=O)C[C@@H](Cc1cnc[nH]1)C(=O)N[C@@H](C)C(=O)N[C@H](C(N)=O)CSC3)C2] |
| 58 | 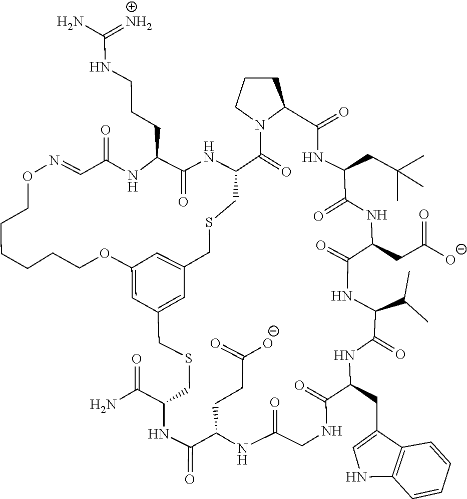 [SMILES: CC(C)[C@@H]1NC(=O)[C@H](CC(=O)[O-])NC(=O)[C@H](CC(C)(C)C)NC(=O)[C@@H]2CCCN2C(=O)[C@@H]2CSCc3cc(cc(c3)OCCCCCCO/N=C/C(=O)N[C@@H](CCCNC(N)=[NH2+])C(=O)N2)CSC[C@@H](C(N)=O)NC(=O)[C@H](CCC(=O)[O-])NC(=O)CNC(=O)[C@H](Cc2c[nH]c3ccccc23)NC1=O] |
| 59 | 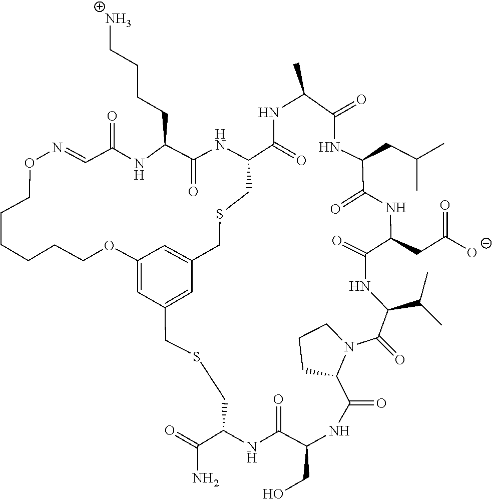 [SMILES: CC(C)C[C@@H]1NC(=O)[C@H](C)NC(=O)[C@@H]2CSCc3cc(cc(c3)OCCCCCCO/N=C/C(=O)N[C@@H](CCCC[NH3+])C(=O)N2)CSC[C@@H](C(N)=O)NC(=O)[C@H](CO)NC(=O)[C@@H]2CCCN2C(=O)[C@H](C(C)C)NC(=O)[C@H](CC(=O)[O-])NC1=O] |
| 60 | 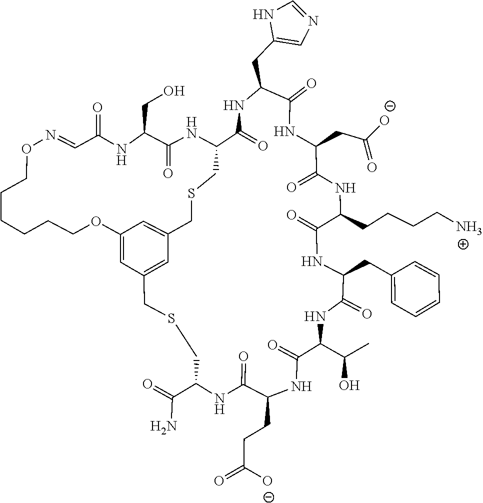 |
| 61 | 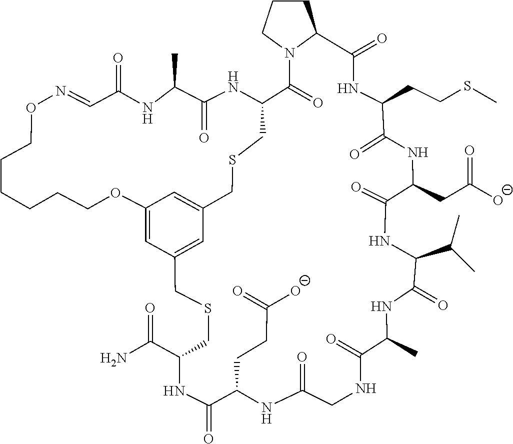 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)CNC(=O)[C@H](C)NC(=O)[C@H](C(C)C)NC(=O)[C@H](CC(=O)[O-])NC1=O] |
| 62 | 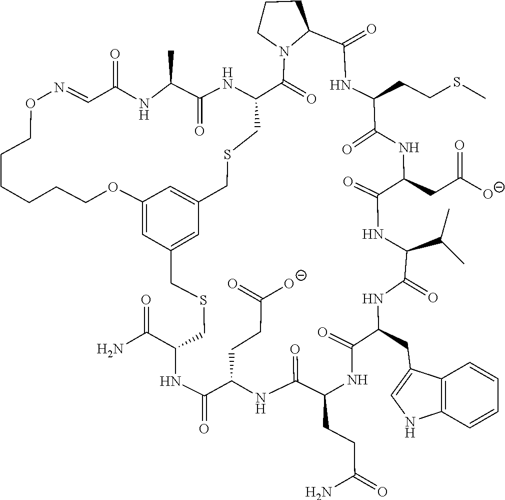 [SMILES: CSCC[C@@H]1NC(=O)[C@@H]2CCCN2C(=O)[C@@H]2CSCc3cc(cc(c3)OCCCCCCO/N=C/C(=O)N[C@@H](C)C(=O)N2)CSC[C@@H](C(N)=O)NC(=O)[C@H](CCC(=O)[O-])NC(=O)[C@H](CCC(N)=O)NC(=O)[C@H](Cc2c[nH]c3ccccc23)NC(=O)[C@H](C(C)C)NC(=O)[C@H](CC(=O)[O-])NC1=O] |
| 63 | 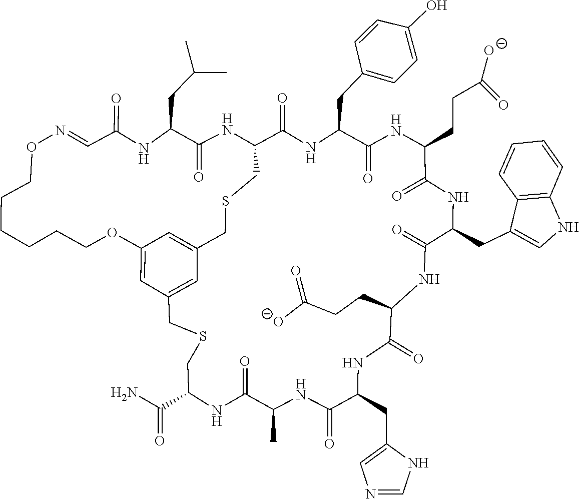 [SMILES: CC(C)C[C@@H]1NC(=O)/C=N/OCCCCCCOc2cc3cc(c2)CSC[C@H](NC1=O)C(=O)N[C@@H](Cc1ccc(O)cc1)C(=O)N[C@@H](CCC(=O)[O-])C(=O)N[C@@H](Cc1c[nH]c2ccccc12)C(=O)N[C@H](CCC(=O)[O-])C(=O)N[C@@H](Cc1cnc[nH]1)C(=O)N[C@@H](C)C(=O)N[C@H](C(N)=O)CSC3] |

29. (canceled)

30. A pharmaceutical composition comprising a compound of claim 1 or a pharmaceutically acceptable salt thereof.

31. A method for treating a disease or condition associated with biological function of α4β7 comprising administering to a subject in need thereof an effective amount of the pharmaceutical composition of claim 30.

32.-33. (canceled)

34. The method of claim 33, wherein the disease comprises inflammatory bowel disease, ulcerative colitis, Crohn's disease, Celiac disease, colitis, diverticulitis, eosinophilic gastroenteritis, gastrointestinal cancer, autoimmune hepatitis, pancreatitis, encephalomyelitis, psoriasis and other inflammatory dermatoses such as dermatitis, eczema, atopic dermatitis, allergic contact dermatitis, urticarial, and pruritus, autoimmune diseases, such as fibromyalgia, scleroderma, ankylosing spondylitis, juvenile rheumatoid arthritis (RA), Still's disease, polyarticular juvenile RA, pauciarticular juvenile RA, polymyalgia rheumatica, RA, psoriatic arthritis, osteoarthritis, polyarticular arthritis, multiple sclerosis, lupus, systemic lupus erythematosus, type I diabetes (T1DM), type diabetes (T2DM), glomerulonephritis, graft-v-host disease (including both acute and chronic), and HIV infection.

35.-42. (canceled)
